(19) United States
(12) Patent Application Publication
(10) Pub. No.: US 2005/0119266 A1
(43) Pub. Date: Jun. 2, 2005

(54) Pyrrolidine and piperidine derivatives as factor Xa inhibitors

(76) Inventors: Yan Shi, Flourtown, PA (US); Philip D. Stein, Pennington, NJ (US); Wei Han, Yardley, PA (US); Timur Gungor, Pennington, NJ (US)

Correspondence Address: STEPHEN B. DAVIS;BRISTOL-MYERS SQUIBB COMPANY, PATENT DEPARTMENT, P O BOX 4000, PRINCETON, NJ, 08543-4000, US

(21) Appl. No.: 10/952,204

(22) Filed: Sep. 28, 2004

Related U.S. Application Data
(60) Provisional application No. 60/507,533, filed on Oct. 1, 2003.

(51) Int. Cl. A61K 31/517; A61K 31/502; A61K 31/498; A61K 31/454; C07D 43/02

(52) U.S. Cl. 514/248; 514/249; 514/266.22; 514/266.2; 514/320; 544/235; 544/284; 544/353; 546/176; 546/198; 546/199; 514/314

(57) ABSTRACT

The present application describes pyrrolidine and piperidine derivatives or pharmaceutically acceptable salt forms thereof. Compounds of the present invention are useful as inhibitors of trypsin-like serine proteases, specifically factor Xa.

### CROSS-REFERENCE TO RELATED APPLICATIONS

The present application claims the priority benefit of U.S. Provisional Application No. 60/507,533, filed Oct. 1, 2003, which is expressly incorporated fully herein by reference.

### FIELD OF THE INVENTION

This invention relates generally to pyrrolidine and piperidine derivatives that are inhibitors of trypsin-like serine protease enzymes, especially factor Xa, pharmaceutical compositions containing the same, and methods of using the same as anticoagulant agents for treatment of thromboembolic disorders.

### BACKGROUND OF THE INVENTION

Activated factor Xa, whose major practical role is the generation of thrombin by the limited proteolysis of prothrombin, holds a central position that links the intrinsic and extrinsic activation mechanisms in the final common pathway of blood coagulation. The generation of thrombin, the final serine protease in the pathway to generate a fibrin clot, from its precursor is amplified by formation of prothrombinase complex (factor Xa, factor V, Ca²⁺ and phospholipid). Since it is calculated that one molecule of factor Xa can generate 138 molecules of thrombin (Elodi, S., Varadi, K.: Optimization of conditions for the catalytic effect of the factor IXa-factor VIII Complex: Probable role of the complex in the amplification of blood coagulation. Thromb. Res. 1979, 15, 617-629), inhibition of factor Xa may be more efficient than inactivation of thrombin in interrupting the blood coagulation system.

Therefore, efficacious and specific inhibitors of factor Xa are needed as potentially valuable therapeutic agents for the treatment of thromboembolic disorders. It is thus desirable to discover new factor Xa inhibitors. In addition, it is also desirable to find new compounds with improved pharmacological characteristics compared with known factor Xa inhibitors. For example, it is preferred to find new compounds with improved factor Xa inhibitory activity and selectivity for factor Xa versus other serine proteases (i.e., trypsin). It is also desirable and preferable to find compounds with advantageous and improved characteristics in one or more of the following categories, but are not limited to: (a) pharmaceutical properties; (b) dosage requirements; (c) factors which decrease blood concentration peak-to-trough characteristics; (d) factors that increase the concentration of active drug at the receptor; (e) factors that decrease the liability for clinical drug-drug interactions; (f) factors that decrease the potential for adverse side-effects; and (g) factors that improve manufacturing costs or feasibility.

### SUMMARY OF THE INVENTION

Accordingly, the present invention provides novel pyrrolidine and piperidine derivatives that are useful as factor Xa inhibitors or pharmaceutically acceptable salts or prodrugs thereof.

The present invention also provides novel processes and intermediates for making the compounds of the present invention or pharmaceutically acceptable salt or prodrug forms thereof.

The present invention provides pharmaceutical compositions comprising a pharmaceutically acceptable carrier and a therapeutically effective amount of at least one of the compounds of the present invention or a pharmaceutically acceptable salt or prodrug form thereof.

The present invention provides a method for treating thromboembolic disorders comprising administering to a host in need of such treatment a therapeutically effective amount of at least one of the compounds of the present invention or a pharmaceutically acceptable salt or prodrug form thereof.

The present invention provides a novel method of treating a patient in need of thromboembolic disorder treatment, comprising: administering a compound of the present invention or a pharmaceutically acceptable salt form thereof in an amount effective to treat a thromboembolic disorder.

The present invention provides a novel method, comprising: administering a compound of the present invention or a pharmaceutically acceptable salt form thereof in an amount effective to treat a thromboembolic disorder.

The present invention provides novel compounds and derivatives thereof for use in therapy.

The present invention provides the use of novel compounds for the manufacture of a medicament for the treatment of a thromboembolic disorder.

These and other provisions, which will become apparent during the following detailed description, have been achieved by the inventors' discovery that the compounds as defined below or pharmaceutically acceptable salt or prodrug forms thereof, are effective factor Xa inhibitors.

### DETAILED DESCRIPTION OF PREFERRED EMBODIMENTS

In a first embodiment, the present invention provides a novel compound of formula Ia or Ib: 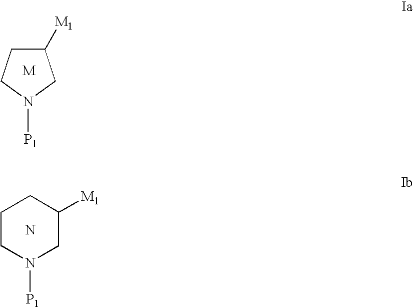
or a stereoisomer or pharmaceutically acceptable salt thereof, wherein;
rings M and N are substituted with 0-3 R^{1a}, 0-2 R³, and 0-1 double bonds;
one of P₁ and M₁ is -Z-A-B and the other -G₁-G;
G is a group of formula Ia or IIb: 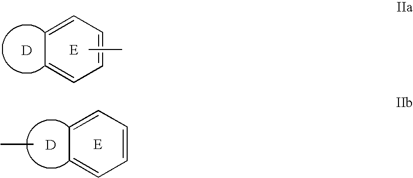
ring D, including the two atoms of ring E to which it is attached, is a 5-6 membered ring consisting of: carbon atoms and 0-2 heteroatoms selected from the group consisting of N, O, and S(O)ₚ;
ring D is substituted with 0-2 R and there are 0-3 ring double bonds;
E is selected from phenyl, pyridyl, pyrimidyl, pyrazinyl, and pyridazinyl, and is substituted with 1-3 R;
alternatively, ring D is absent, and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1-3 R;
alternatively, ring D is absent, ring E is selected from phenyl, phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1 R and either phenyl or a 5-6 membered heterocycle consisting of: carbon atoms and 1-4 heteroatoms selected from the group consisting of N, O, and S(O)ₚ, wherein the phenyl is substituted with 1-2 R and the 5-6 membered heterocycle is substituted with 0-2 carbonyls and 1-2 R and has 0-3 ring double bonds;
R is selected from H, C₁₋₄ alkyl, F, Cl, Br, I, OH, OCH₃, OCH₂CH₃, OCH(CH₃)₂, OCH₂CH₂CH₃, CN, C(═NR⁸)NR⁷R⁹, NHC(═NR⁸)NR⁷R⁹, NR⁸CH(═NR⁷), NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)₂, (CR⁸R⁹)ₜC(O)H, (CR⁸R⁹)ₜC(O)R^{2c}, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜOR³, (CR⁸R⁹)ₜS(O)ₚNR⁷R⁸, (CR⁸R⁹)ₜNR⁷S(O)ₚR⁷, (CR⁸R⁹)ₜSR³, (CR⁸R⁹)ₜS(O)R³, (CR⁸R⁹)ₜS(O)₂R³, and OCF₃;
alternatively, when 2 R groups are attached to adjacent atoms, they combine to form methylenedioxy or ethylenedioxy;
A is selected from: C₃₋₁₀ carbocycle substituted with 0-2 R⁴, and 5-12 membered heterocycle substituted with 0-2 R⁴ and consisting of: carbon atoms and 1-4 heteroatoms selected from the group consisting of N, O, and S(O)ₚ;
B is selected from: Y, X—Y, (CH₂)₀₋₂C(O)NR²R^{2a}, and (CH₂)₀₋₂NR²R^{2a}, provided that Z and B are attached to different atoms on A;
X is selected from —(CR²R^{2a})₁₋₄—, —CR²(CR²R^{2b})(CH₂)ₜ—, —C(O)—, —C(═NR^{1b})—, —CR²(NR^{1b}R²)—, —CR²(OR²)—, —CR²(SR²)—, —C(O)CR²R^{2a}—, —CR²R^{2a}C(O), —S(O)—, —S(O)₂—, —SCR²R^{2a}—, —S(O)CR²R^{2a}—, —S(O)₂CR²R^{2a}—, —CR²R^{2a}S—, —CR²R^{2a}S(O)—, —CR²R^{2a}S(O)₂—, —S(O)₂NR²—, —S(O)₂NR²CR²R^{2a}—, —CR²R^{2a}S(O)₂NR²—, —NR²S(O)₂—, —CR²R^{2a}NR²S(O)₂—, —NR²S(O)₂CR²R^{2a}—, —NR²C(O)—, —C(O)NR²—, —NR²C(O)CR²R^{2a}—, —C(O)NR²CR²R^{2a}—, —CR²R^{2a}NR²C(O)—, —CR²R^{2a}C(O)NR²—, NR², —NR²CR²R^{2a}—, —CR²R^{2a}NR²—, O, —OCR²R^{2a}—, and —CR²R^{2a}O—;
Y is selected from: C₃₋₁₀ carbocycle substituted with 0-2 R^{4a}, and 5-10 membered heterocycle consisting of: carbon atoms and 1-4 heteroatoms selected from the group consisting of N, O, and S(O)ₚ substituted with 0-2 R^{4a};
G₁ is selected from ═O or a 2-6 membered linear chain consisting of: 0-6 carbon atoms, 0-2 carbonyl groups, and 0-3 heteroatoms selected from O, N, and S(O)ₚ, and G₁ is substituted with 0-4 R³, and there are 0-2 double bonds and 0-1 triple bond; provided that other than an S—S, S—O, or O—O bond is present in G₁;
Z is selected from —CR³R^{3e})₁₋₄—, (CR³R^{3e})_{q}O(CR³R^{3e})_{q1}, (CR³R^{3e})_{q}NR^{3b}(CR³R^{3e})_{q1}, (CR³R^{3e})_{q}C(O)(CR³R^{3e})_{q1}, (CR³R^{3e})_{q}C(O)O(CR³R^{3e})_{q1}, (CR³R^{3e})_{q}OC(O)(CR³R^{3e})_{q1}, —(CR³R^{3e})_{q}C(O)NR^{3b}(CR³R^{3e})_{q1}, (CR³R^{3e})_{q}NR^{3b}C(O)(CR³R^{3e})_{q1}, (CR³R^{3e})_{q}OC(O)O(CR³R^{3e})_{q1}, (CR³R^{3e})_{q}OC(O)NR^{3b}(CR³R^{3e})_{q1}, (CR³R^{3e})_{q}NR^{3b}C(O)O(CR³R^{3e})_{q1}, (CR³R^{3e})_{q}NR^{3b}C(O)NR^{3b}(CR³R^{3e})_{q1}, (CR³R^{3e})_{q}C(O)(CR³R^{3e})_{q}C(O)(CR³R^{3e})_{q}, (CR³R^{3e})_{q}NR^{3b}(CR³R^{3e})_{q}C(O)NR^{3b}(CR³R^{3e})_{q1}, (CR³R^{3e})_{q}NR^{3b}C(O)(CR³R^{3e})_{q}C(O)(CR³R^{3e})_{q1}, (CR³R^{3e})_{q}C(O)(CR³R^{3e})_{q}C(O)NR^{3b}(CR³R^{3e})_{q1}, (CR³R^{3e})_{q}NR^{3b}C(O)(CR³R^{3e})_{q}C(O)NR^{3b}(CR³R^{3e})_{q1}, (CR³R^{3e})_{q}S(CR³R^{3e} (CR³R^{3e})_{q}S(O)(CR³R^{3e})_{q1}, (CR³R^{3e})_{q}S(O)₂(CR³R^{3e})_{q1}, (CR³R^{3e})_{q}SO₂NR^{3b}(CR³R^{3e})_{q1}, (CR³R^{3e})_{q}NR^{3b}SO₂(CR³R^{3e})_{q1}, (CR³R^{3e})_{q}S(O)NR^{3b}C(O)(CR³R^{3e})_{q1}, (CR³R^{3e})_{q}C(O)NR^{3b}S(O)₂(CR³R^{3e})_{q1}, and (CR³R^{3e})_{q}NR^{3b}SO₂NR^{3b}(CR³R^{3e})_{q1}, wherein q+q1 total 0, 1, 2, 3, or 4, provided that Z does not form a N—S, NCH₂N, NCH₂O, or NCH₂S bond with either group to which it is attached;
R^{1a}, at each occurrence, is selected from H, —(CR³R^{3a})ᵣR^{1b}, —(CR³R^{3a})ᵣ—CR³R^{1b}R^{1b}, —(CR³R^{3a})ᵣ—O—(CR³R^{3a})ᵣ—R^{1b}, —(CR³R^{3a})ᵣ—NR²—(CR³R^{3a})ᵣ—R^{1b}, —(CR³R^{3a})ᵣ—S(O)ₚ—(CR³R^{3a})ᵣ—R^{1b}, —(CR³R^{3a})ᵣ—CO₂—(CR³R^{3a})ᵣ—R^{1b}, —(CR³R^{3a})ᵣ—C(O)NR²—(CR³R^{3a})ᵣ—R^{1b}, —(CR³R^{3a})ᵣ—C(O)—(CR³R^{3a})ᵣ—R^{1b}, —C₂₋₆ alkenylene-R^{1b}, —C₂₋₆ alkynylene-R^{1b}, and —(CR³R^{3a})ᵣ—C(═NR^{1b})NR³R^{1b}, provided that R^{1a} forms other than an N-halo, N—S, O—O, or N—CN bond;
provided that R^{1a} is other than a substituted or unsubstituted 3,4-dihydroxyphenyl group;
alternatively, when two R^{1a} groups are attached to adjacent atoms or to the same carbon atom, together with the atoms to which they are attached, they form a 5-7 membered ring consisting of: carbon atoms and 0-2 heteroatoms selected from the group consisting of N, O, and S(O)ₚ, this ring being substituted with 0-2 R^{4b} and comprising: 0-3 ring double bonds;
R^{1b} is selected from H, C₁₋₃ alkyl, F, Cl, Br, I, —CN, —NO₂, (CF₂)ᵣCF₃, (CR³R^{3a})ᵣOR², NR²R^{2a}, C(O)R^{2b}, CO₂R^{2b}, OC(O)R², CH(CH₂OR²)₂, (CF₂)ᵣCO₂R^{2a}, S(O)ₚR^{2b}, NR²(CH₂)ᵣOR², C(═NR^{2c})NR²R^{2a}, NR²C(O)R^{2b}, NR²C(O)NR²R^{2a}, NR²C(O)₂R^{2a}, OC(O)NR²R^{2a}, C(O)NR²R^{2a}, C(O)NR²(CH₂)ᵣOR², SO₂NR²R^{2a}, NR²SO₂R², C(O)NR²SO₂R², C₃₋₆ carbocycle substituted with 0-2 R^{4b}, and 5-10 membered heterocycle substituted with 0-2 R^{4b} and consisting of carbon atoms and from 1-4 heteroatoms selected from the group consisting of N, O, and S(O)ₚ, provided that R^{1b} forms other than an O—O, N-halo, N—S, or N—CN bond and provided that S(O)ₚR² forms other than S(O)₂H or S(O)H;
R², at each occurrence, is selected from H, CF₃, C₁₋₆ alkyl, benzyl, —(CH₂)ᵣ—C₃₋₁₀ carbocycle substituted with 0-2 R^{4b}, and —(CH₂)ᵣ-5-10 membered heterocycle substituted with 0-2 R^{4b} and consisting of: carbon atoms and 1-4 heteroatoms selected from the group consisting of N, O, and S(O)ₚ;
R^{2a}, at each occurrence, is selected from H, CF₃, C₁₋₆ alkyl, benzyl, —(CH₂)ᵣC₃₋₁₀ carbocycle substituted with 0-2 R^{4b}, and —(CH₂)ᵣ₋5-10 membered heterocycle substituted with 0-2 R^{4b} and consisting of: carbon atoms and 1-4 heteroatoms selected from the group consisting of N, O, and S(O)ₚ;
alternatively, NR²R^{2a} forms a 5 or 6 membered saturated, partially saturated or unsaturated ring substituted with 0-2 R^{4b} and consisting of: 0-1 additional heteroatoms selected from the group consisting of N, O, and S(O)ₚ;
R^{2b}, at each occurrence, is selected from CF₃, C₁₋₄ alkoxy substituted with 0-2 R^{4b}, C₁₋₆ alkyl substituted with 0-2 R^{4b}, —(CH₂)ᵣ—C₃₋₁₀ carbocycle substituted with 0-2 R^{4b}, and —(CH₂)ᵣ-5-10 membered heterocycle substituted with 0-2 R^{4b} and consisting of: carbon atoms and 1-4 heteroatoms selected from the group consisting of N, O, and S(O)ₚ;
R^{2c}, at each occurrence, is selected from CF₃, OH, C₁₋₄ alkoxy, C₁₋₆ alkyl, —(CH₂)ᵣ—C₃₋₁₀ carbocycle substituted with 0-2 R^{4b}, and —(CH₂)ᵣ-5-10 membered heterocycle substituted with 0-2 R^{4b} and consisting of carbon atoms and from 1-4 heteroatoms selected from the group consisting of N, O, and S(O)ₚ;
R³, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
R^{3a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
alternatively, NR³R^{3a} forms a 5 or 6 membered saturated, partially unsaturated, or unsaturated ring consisting of: carbon atoms, the nitrogen atom to which R³ and R^{3a} are attached, and 0-1 additional heteroatoms selected from the group consisting of N, O, and S(O)ₚ;
R^{3b}, at each occurrence, is selected from H, C₁₋₆ alkyl substituted with 0-2 R^{1a}, C₂₋₆ alkenyl substituted with 0-2 R^{1a}, C₂₋₆ alkynyl substituted with 0-2 R^{1a}, —(C₀₋₄ alkyl)-5-10 membered carbocycle substituted with 0-3 R^{1a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;
R^{3c}, at each occurrence, is selected from CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
R^{3d}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C₁₋₄ alkyl-phenyl, and C(═O)R^{3c};
R^{3e}, at each occurrence, is selected from H, S(O)₂NHR³, C(O)R³, C(O)NHR³, C(O)OR^{3f}, S(O)R^{3f}, S(O)₂R^{3f}, C₁₋₆ alkyl substituted with 0-2 R^{1a}, C₂₋₆ alkenyl substituted with 0-2 R^{1a}, C₂₋₆ alkynyl substituted with 0-2 R^{1a}, —(C₀₋₄ alkyl)-5-10 membered carbocycle substituted with 0-3 R^{1a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;
R^{3f}, at each occurrence, is selected from: C₁₋₆ alkyl substituted with 0-2 R^{1a}, C₂₋₆ alkenyl substituted with 0-2 R^{1a}, C₂₋₆ alkynyl substituted with 0-2 R^{1a}, —(C₀₋₄ alkyl)-5-10 membered carbocycle substituted with 0-3 R^{a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;
R⁴, at each occurrence, is selected from H, ═O, (CR³R^{3a})ᵣOR², F, Cl, Br, I, C₁₋₄ alkyl, (CR³R^{3a})ᵣCN, (CR³R^{3a})ᵣNO₂, (CR³R^{3a})ᵣNR²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣNR²C(O)NR²R^{2a}, (CR³R^{3a})ᵣC(═NR²)NR²R^{2a}, (CR³R^{3a})ᵣC(═NS(O)₂R^{5a})NR²R^{2a}, (CR³R^{3a})ᵣNR²C(═NR²)NR²R^{2a}, (CR³R^{3a})ᵣC(O)NR²C(═NR²)NR²R^{2a}, (CR³R^{3a})ᵣSO₂NR²R^{2a}, (CR³R^{3a})ᵣNR²SO₂NR²R^{2a}, (CR³R^{3a})ᵣNR²SO₂—C₁₋₄ alkyl, (CR³R^{3a})ᵣNR²SO₂R^{5a}, (CR³R^{3a})ᵣS(O)ₚR^{5a}, (CR³R^{3a})ᵣ(CF₂)ᵣCF₃, N(CH₂)ᵣR^{1b}, O(CH₂)ᵣR^{1b}, S(CH₂)ᵣR^{1b}, (CR³R^{3a})ᵣ-5-6 membered carbocycle substituted with 0-1 R⁵, and a (CR³R^{3a})ᵣ-5-6 membered heterocycle substituted with 0-1 R⁵ and consisting of: carbon atoms and 1-4 heteroatoms selected from the group consisting of N, O, and S(O)ₚ;
R^{4a}, at each occurrence, is selected from H, ═O, (CR³R^{3a})ᵣOR², (CR³R^{3a})ᵣF, (CR³R^{3a})ᵣBr, (CR³R^{3a})ᵣCl, (CR³R^{3a})ᵣI, C₁₋₄ alkyl, (CR³R^{3a})ᵣCN, (CR³R^{3a})ᵣNO₂, (CR³R^{3a})ᵣNR²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣN═CHOR³, (CR³R^{3a})ᵣC(O)NH(CH₂)₂NR²R^{2a}, (CR³R^{3a})ᵣNR²C(O)NR²R^{2a}, (CR³R^{3a})ᵣNR²C(O)OR², (CR³R^{3a})ᵣC(═NR²)NR²R^{2a}, (CR³R^{3a})ᵣNHC(═NR²)NR²R^{2a}, (CR³R^{3a})ᵣSO₂NR²R^{2a}, (CR³R^{3a})ᵣNR²SO₂NR²R^{2a}, (CR³R^{3a})ᵣNR²SO₂—C₁₋₄ alkyl, (CR³R^{3a})ᵣC(O)NHSO₂—C₁₋₄ alkyl, (CR³R^{3a})ᵣNR²SO₂R⁵, (CR³R^{3a})ᵣS(O)ₚR⁵, (CR³R^{3a})ᵣ(CF₂)ᵣCF₃, (CR³R^{3a})ᵣ-3-10 membered carbocycle substituted with 0-1 R⁵, and a (CR³R^{3a})ᵣ-3-10 membered heterocycle consisting of: carbon atoms and 1-4 heteroatoms selected from the group consisting of N, O, and S(O)ₚ and substituted with 0-1 R⁵;
R^{4b}, at each occurrence, is selected from H, ═O, (CH₂)ᵣOR³, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR³R^{3a}, (CH₂)ᵣC(O)R³, (CH₂)ᵣC(O)OR^{3c}, (CH₂)ᵣNR³C(O)R^{3a}, (CH₂)ᵣC(O)NR³R^{3a}, (CH₂)ᵣNR³C(O)NR³R^{3a}, (CH₂)ᵣC(═NR³)NR³R^{3a}, (CH₂)ᵣNR³C(═NR³)NR³R^{3a}, (CH₂)ᵣSO₂NR³R^{3a}, (CH₂)ᵣNR³SO₂NR³R^{3a}, (CH₂)ᵣNR³SO₂—C₁₋₄ alkyl, (CH₂)ᵣNR³SO₂CF₃,(CH₂)ᵣNR³SO₂-phenyl, (CH₂)ᵣS(O)ₚCF₃, (CH₂)ᵣS(O)ₚ—C₁₋₄ alkyl, (CH₂)ᵣS(O)ₚ-phenyl, and (CH₂)ᵣ(CF₂)ᵣCF₃;
R⁵, at each occurrence, is selected from H, C₁₋₆ alkyl, ═O, (CH₂)ᵣOR³, F, Cl, Br, I, —CN, NO₂, (CH₂)ᵣNR³R^{3a}, (CH₂)ᵣC(O)R³, (CH₂)ᵣC(O)OR^{3c}, (CH₂)ᵣNR³C(O)R^{3a}, (CH₂)ᵣC(O)NR³R^{3a}, (CH₂)ᵣNR³C(O)NR³R^{3a}, (CH₂)ᵣCH(═NOR^{3d}), (CH₂)ᵣC(═NR³)NR³R^{3a}, (CH₂)ᵣNR³C(═NR³)NR³R^{3a}, (CH₂)ᵣSO₂NR³R^{3a}, (CH₂)ᵣNR³SO₂NR³R^{3a}, (CH₂)ᵣNR³SO₂—C₁₋₄ alkyl, (CH₂)ᵣNR³SO₂CF₃, (CH₂)ᵣNR³SO₂-phenyl, (CH₂)ᵣS(O)ₚCF₃, (CH₂)ᵣS(O)ₚ—C₁₋₄ alkyl, (CH₂)ᵣS(O)ₚ-phenyl, (CF₂)ᵣCF₃, phenyl substituted with 0-2 R⁶, naphthyl substituted with 0-2 R⁶, and benzyl substituted with 0-2 R⁶;
R^{5a}, at each occurrence, is selected from C₁₋₆ alkyl, (CH₂)ᵣOR³, (CH₂)ᵣNR³R^{3a}, (CH₂)ᵣC(O)R³, (CH₂)ᵣC(O)OR^{3c}, (CH₂)ᵣNR³C(O)R^{3a}, (CH₂)ᵣC(O)NR³R^{3a}, (CF₂)ᵣCF₃, phenyl substituted with 0-2 R⁶, naphthyl substituted with 0-2 R⁶, and benzyl substituted with 0-2 R⁶, provided that R^{5a} does not form a S—N or S(O)ₚ—C(O) bond;
R⁶, at each occurrence, is selected from H, OH, (CH₂)ᵣOR², halo, C₁₋₄ alkyl, —CN, NO₂, (CH₂)ᵣNR²R^{2a}, (CH₂)ᵣC(O)R^{2b}, NR²C(O)R^{2b}, NR²C(O)NR²R^{2a}, C(═NH)NH₂, NHC(═NH)NH₂, SO₂NR²R^{2a}, NR²SO₂NR²R^{2a}, and NR²SO₂C₁₋₄ alkyl;
R⁷, at each occurrence, is selected from H, OH, C₁₋₆ alkyl, C₁₋₆ alkyl-C(O)—, C₁₋₆ alkyl-O—, (CH₂)ₙ-phenyl, C₁₋₆ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl C₀₋₄ alkyl-C(O)—;
R⁸, at each occurrence, is selected from H, C₁₋₆ alkyl, and (CH₂)ₙ-phenyl;
alternatively, NR⁷R⁸ forms a 5-10 membered heterocyclic ring consisting of carbon atoms and 0-2 additional heteroatoms selected from the group consisting of N, O, and S(O)ₚ;
R⁹, at each occurrence, is selected from H, C₁₋₆ alkyl, and (CH₂)ₙ-phenyl;
n, at each occurrence, is selected from 0, 1, 2, and 3;
p, at each occurrence, is selected from 0, 1, and 2;
r, at each occurrence, is selected from 0, 1, 2, 3, 4, 5, and 6; and
t, at each occurrence, is selected from 0, 1, 2, and 3.

Preferably, G₁ is other than C(O)NR³ when attached to the nitrogen atom of Ring M (wherein the carbonyl is directly attached to the ring nitrogen).

Preferably, Z is other than C(O)NR^{3b} when attached to the nitrogen atom of Ring M (wherein the carbonyl is directly attached to the ring nitrogen).

In a second embodiment, the present invention provides a novel compound, wherein:
rings M and N are substituted with 0-2 R^{1a} and 0-2 R³;
one of P₁ and M₁ is -Z-A-B and the other -G₁-G;
G is a group of formula IIa or IIb: 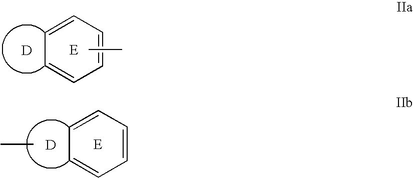
ring D, including the two atoms of ring E to which it is attached, is a 5-6 membered ring consisting of: carbon atoms and 0-2 heteroatoms selected from the group consisting of N, O, and S(O)ₚ;
ring D is substituted with 0-2 R and there are 0-3 ring double bonds;
E is selected from phenyl, pyridyl, pyrimidyl, pyrazinyl, and pyridazinyl, and is substituted with 1-2 R;
alternatively, ring D is absent, and ring E is selected from phenyl, pyridyl, pyrimidyl, and thienyl, and ring E is substituted with 1-2 R;
alternatively, ring D is absent, ring E is selected from phenyl, pyridyl, and thienyl, and ring E is substituted with either phenyl or a 5-6 membered heterocycle consisting of: carbon atoms and 1-4 heteroatoms selected from the group consisting of N, O, and S(O)ₚ, wherein the phenyl is substituted with 1-2 R and the 5-6 membered heterocycle is substituted with 0-1 carbonyls and 1-2 R and has 0-3 ring double bonds;
R is selected from H, C₁₋₄ alkyl, F, Cl, OH, OCH₃, OCH₂CH₃, OCH(CH₃)₂, CN, C(═NH)NH₂, NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, (CR⁸R⁹)ₜNR⁷R⁸, C(O)NR⁷R⁸, CH₂C(O)NR⁷R⁸, NHC(O)R⁷, S(O)ₚNR⁷R⁸, CH₂S(O)ₚNR⁷R⁸, and OCF₃;
alternatively, when 2 R groups are attached to adjacent atoms, they combine to form methylenedioxy or ethylenedioxy;
A is selected from: C-10 membered heterocycle substituted with 0-2 R⁴ and consisting of: carbon atoms and 1-4 heteroatoms selected from the group consisting of N, O, and S(O)ₚ;
B is selected from Y, X—Y, CH₂NR²R^{2a}, and CH₂CH₂NR²R^{2a};
X is selected from —CR²R^{2a})₁₋₄—, —C(O)—, —C(O)CR²R^{2a}—, —CR²R^{2a}C(O), —S(O)₂—, —S(O)₂CR²R^{2a}—, —CR²R^{2a}S(O)₂—, —NR²S(O)₂—, —NR²S(O)₂—, —NR²C(O)—, —C(O)NR²—, NR², —NR²CR²R^{2a}—, —CR²R^{2a}NR²—, O, —OCR²R^{2a}—, and —CR²R^{2a}O—;
Y is selected from one of the following rings and is substituted with 0-2 R^{4a}; cyclopropyl, cyclopentyl, cyclohexyl, phenyl, piperidinyl, piperazinyl, pyridyl, pyrimidyl, furanyl, morpholinyl, thiophenyl, pyrrolyl, pyrrolidinyl, oxazolyl, isoxazolyl, isoxazolinyl, thiazolyl, isothiazolyl, pyrazolyl, imidazolyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, 1,2,3-triazolyl, 1,2,4-triazolyl, 1,2,5-triazolyl, 1,3,4-triazolyl, benzofuranyl, benzothiofuranyl, indolyl, benzimidazolyl, benzoxazolyl, benzthiazolyl, indazolyl, benzisoxazolyl, benzisothiazolyl, and isoindazolyl;
alternatively, Y is selected from the following bicyclic heteroaryl ring systems: 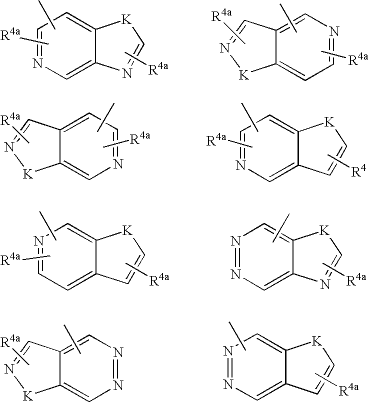
K is selected from O, S, NH, and N;
G₁ is selected from ═O or a 2-5 membered linear chain consisting of: 0-5 carbon atoms, 0-2 carbonyl groups, and 0-3 heteroatoms selected from O, N, and S(O)ₚ, and G₁ is substituted with 0-4 R³, and there are 0-1 double bonds; provided that other than an S—S, S—O, or O—O bond is present in G₁;
Z is selected from —(CR³R^{3e})₂₋₃—, (CR³R^{3e})_{q}C(O)(CR³R^{3e})_{q1}, (CR³R^{3e})_{q}O(CR³R^{3e})_{q1}, (CR³R^{3e})_{q}NR^{3b}(CR³R^{3e})_{q1}, (CR³R^{3e})_{q}C(O)NR^{3b}(CR³R^{3e})_{q1}, (CR³R^{3e})_{q}NR^{3b}C(O)(CR³R^{3e})_{q1}, (CR³R^{3e})_{q}NR^{3b}C(O)NR^{3b}(CR³R^{3e})_{q1}, (CR³R^{3e})_{q}NR^{3b}(CR³R^{3e})_{q}C(O)NR^{3b}(CR³R^{3e})_{q1}, (CR³R^{3e})_{q}S(CR³R^{3e})_{q1}, (CR³R^{3e})_{q}S(O)(CR³R^{3e})_{q1}, (CR³R^{3e})_{q}S(O)₂(CR³R^{3e})_{q1}, (CR³R^{3e})_{q}SO₂NR^{3b}(CR³R^{3e})_{q1}, (CR³R^{3e})_{q}NR^{3b}SO₂(CR³R^{3e})_{q1}, and (CR³R^{3e})_{q}C(O)NR^{3b}S(O)₂(CR³R^{3e})_{q1}, wherein q+q1 total 1 or 2, provided that Z does not form a N—S, NCH₂N, NCH₂O, or NCH₂S bond with either group to which it is attached;
R^{1a}, at each occurrence, is selected from H, —(CH₂)ᵣ—R^{1b}, —(CH(CH₃))ᵣ—R^{1b}, —(C(CH₃)₂)ᵣ—R^{1b}, —O—(CR³R^{3a})ᵣ—R^{1b}, —NR²—(CR³R^{3a})ᵣ—R^{1b}, and —S—(CR³R^{3a})ᵣ—R^{1b}, provided that R^{1a} forms other than an N-halo, N—S, O—O, or N—CN bond;
provided that R^{1a} is other than a substituted or unsubstituted 3,4-dihydroxyphenyl group;
alternatively, when two R^{1a} groups are attached to adjacent atoms or to the same carbon atom, together with the atoms to which they are attached they form a 5-7 membered ring consisting of: carbon atoms and 0-2 heteroatoms selected from the group consisting of N, O, and S(O)ₚ, this ring being substituted with 0-2 R^{4b} and 0-3 ring double bonds;
R^{1b} is selected from H, CH₃, CH₂CH₃, CH₂CH₂CH₃, CH(CH₃)₂, F, Cl, Br, I, —CN, —CF₃, OR², NR²R^{2a}, C(O)R^{2b}, CO₂R^{2b}, OC(O)R², CO₂R^{2a}, S(O)ₚR², NR²(CH₂)ᵣOR², NR²C(O)R^{2b}, NR²C(O)NHR², NR²C(O)₂R^{2a}, OC(O)NR²R^{2a}, C(O)NR²R^{2a}, C(O)NR²(CH₂)ᵣOR², SO₂NR²R^{2a}, NR²SO₂R², C₅₋₆ carbocycle substituted with 0-2 R^{4b}, and 5-6 membered heterocycle consisting of carbon atoms and from 1-4 heteroatoms selected from the group consisting of N, O, and S(O)ₚ and substituted with 0-2 R^{4b}, provided that R^{1b} forms other than an O—O, N-halo, N—S, or N—CN bond;
R², at each occurrence, is selected from H, CF₃, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, C₅₋₆ carbocycle substituted with 0-2 R^{4b}, a C₅₋₆ carbocycle-CH₂-substituted with 0-2 R^{4b}, and 5-6 membered heterocycle substituted with 0-2 R^{4b} and consisting of: carbon atoms and 1-4 heteroatoms selected from the group consisting of N, O, and S(O)ₚ;
R^{2a}, at each occurrence, is selected from H, CF₃, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, C₅₋₆ carbocycle substituted with 0-2 R^{4b}, and 5-6 membered heterocycle substituted with 0-2 R^{4b} and consisting of: carbon atoms and 1-4 heteroatoms selected from the group consisting of N, O, and S(O)ₚ;
alternatively, NR²R^{2a} forms a 5 or 6 membered saturated, partially saturated or unsaturated ring substituted with 0-2 R^{4b} and consisting of: 0-1 additional heteroatoms selected from the group consisting of N, O, and S(O)ₚ;
R^{2b}, at each occurrence, is selected from CF₃, C₁₋₄ alkoxy, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, C₅₋₆ carbocycle substituted with 0-2 R^{4b}, and 5-6 membered heterocycle substituted with 0-2 R^{4b} and consisting of: carbon atoms and 1-4 heteroatoms selected from the group consisting of N, O, and S(O)ₚ;
R^{2c}, at each occurrence, is selected from CF₃, OH, C₁₋₄ alkoxy, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, C₅₋₆ carbocycle substituted with 0-2 R^{4b}, and 5-6 membered heterocycle substituted with 0-2 R^{4b} and consisting of carbon atoms and from 1-4 heteroatoms selected from the group consisting of N, O, and S(O)ₚ;
R³, at each occurrence, is selected from H, CH₃, CH₂CH₃, CH₂CH₂CH₃, CH(CH₃)₂, benzyl, and phenyl;
R^{3a}, at each occurrence, is selected from H, CH₃, CH₂CH₃, CH₂CH₂CH₃, CH(CH₃)₂, benzyl, and phenyl;
alternatively, NR³R^{3a} forms a 5 or 6 membered saturated, partially unsaturated, or unsaturated ring consisting of: carbon atoms and the nitrogen atom to which R³ and R^{3a} are attached;
R^{3c}, at each occurrence, is selected from CH₃, CH₂CH₃, CH₂CH₂CH₃, CH(CH₃)₂, benzyl, and phenyl;
R^{3d}, at each occurrence, is selected from H, CH₃, CH₂CH₃, CH₂CH₂CH₃, CH(CH₃)₂, CH₂-phenyl, CH₂CH₂-phenyl, and C(═O)R^{3c};
R⁴, at each occurrence, is selected from H, ═O, OR², CH₂OR², (CH₂)₂OR², F, Cl, Br, I, C₁₋₄ alkyl, —CN, NO₂, NR²R^{2a}, CH₂NR²R^{2a}, (CH₂)₂NR²R^{2a}, C(O)R^{2c}, NR²C(O)R^{2b}, C(O)NR²R^{2a}, SO₂NR²R^{2a}, S(O)ₚR^{5a}, CF₃, CF₂CF₃, 5-6 membered carbocycle substituted with 0-1 R⁵, and a 5-6 membered heterocycle substituted with 0-1 R⁵ and consisting of: carbon atoms and 1-4 heteroatoms selected from the group consisting of N, O, and S(O)ₚ;
R^{4a}, at each occurrence, is selected from H, ═O, (CR³R^{3a})ᵣOR², (CR³R^{3a})ᵣ—F, (CR³R^{3a})ᵣ—Br, (CR³R^{3a})ᵣ—Cl, C₁₋₄ alkyl, (CR³R^{3a})ᵣ—CN, (CR³R^{3a})ᵣNO₂, (CR³R^{3a})ᵣNR²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣSO₂NR²R^{2a}, (CR³R^{3a})ᵣNR²SO₂NR²R^{2a}, (CR³R^{3a})ᵣNR²SO₂—C₁₋₄ alkyl, (CR³R^{3a})ᵣC(O)NHSO₂—C₁₋₄ alkyl, (CR³R^{3a})ᵣNR²SO₂R⁵, (CR³R^{3a})ᵣS(O)ₚR⁵, (CR³R^{3a})ᵣ(CF₂)ᵣCF₃, phenyl substituted with 0-1 R⁵, and a 5 membered aromatic heterocycle consisting of: carbon atoms and 1-3 heteroatoms selected from the group consisting of N, O, and S(O)ₚ substituted with 0-1 R⁵;
R^{4b}, at each occurrence, is selected from H, ═O, OR³, CH₂OR³, F, Cl, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —CN, NO₂, NR³R^{3a}, CH₂NR³R^{3a}, C(O)R³, CH₂—C(O)R³, C(O)OR^{3c}, CH₂C(O)OR^{3c}, NR³C(O)R^{3a}, CH₂NR³C(O)R^{3a}, C(O)NR³R^{3a}, CH₂C(O)NR³R^{3a}, NR³C(O)NR³R^{3a}, CH₂NR³C(O)NR³R^{3a}, C(═NR³)NR³R^{3a}, CH₂C(═NR³)NR³R^{3a}, NR³C(═NR³)NR³R^{3a}, CH₂NR³C(═NR³)NR³R^{3a}, SO₂NR³R^{3a}, CH₂SO₂NR³R^{3a}, NR³SO₂NR³R^{3a}, CH₂NR³SO₂NR³R^{3a}, NR³SO₂—C₁₋₄ alkyl, CH₂NR³SO₂—C₁₋₄ alkyl, NR³SO₂CF₃, CH₂NR³SO₂CF₃, NR³SO₂-phenyl, CH₂NR³ SO₂-phenyl, S(O)ₚCF₃, CH₂S(O)ₚCF₃, S(O)ₚ—C₁₋₄ alkyl, CH₂S(O)ₚ—C₁₋₄ alkyl, S(O)ₚ-phenyl, CH₂S(O)ₚ-phenyl, CF₃, and CH₂—CF₃;
R⁵, at each occurrence, is selected from H, ═O, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, OR³, CH₂OR³, F, Cl, —CN, NO₂, NR³R^{3a}, CH₂NR³R^{3a}, C(O)R³, CH₂C(O)R³, C(O)OR^{3c}, CH₂C(O)OR^{3c}, NR³C(O)R^{3a}, C(O)NR³R^{3a}, NR³C(O)NR³R^{3a}, CH(═NOR^{3d}), C(═NR³)NR³R^{3a}, NR³C(═NR³)NR³R^{3a}, SO₂NR³R^{3a}, NR³SO₂NR³R^{3a}, NR³SO₂—C₁₋₄ alkyl, NR³SO₂CF₃, NR³SO₂-phenyl, S(O)ₚCF₃, S(O)ₚ—C₁₋₄ alkyl, S(O)ₚ-phenyl, CF₃, phenyl substituted with 0-2 R⁶, naphthyl substituted with 0-2 R⁶, and benzyl substituted with 0-2 R⁶;
R⁶, at each occurrence, is selected from H, OH, OR², F, Cl, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —CN, NO₂, NR²R^{2a}, CH₂NR²R^{2a}, C(O)R^{2b}, CH₂C(O)R^{2b}, NR²C(O)R^{2b}, NR²C(O)NR²R^{2a}, C(═NH)NH₂, NHC(═NH)NH₂, SO₂NR²R^{2a}, NR²SO₂NR²R^{2a}, and NR²SO₂C₁₋₄ alkyl; and
r, at each occurrence, is selected from 0, 1, 2, and 3.

In a third embodiment, the present invention provides a novel compound, wherein:
rings M and N are substituted with 0-1 R^{1a} and 0-2 R³;
G is selected from the group: 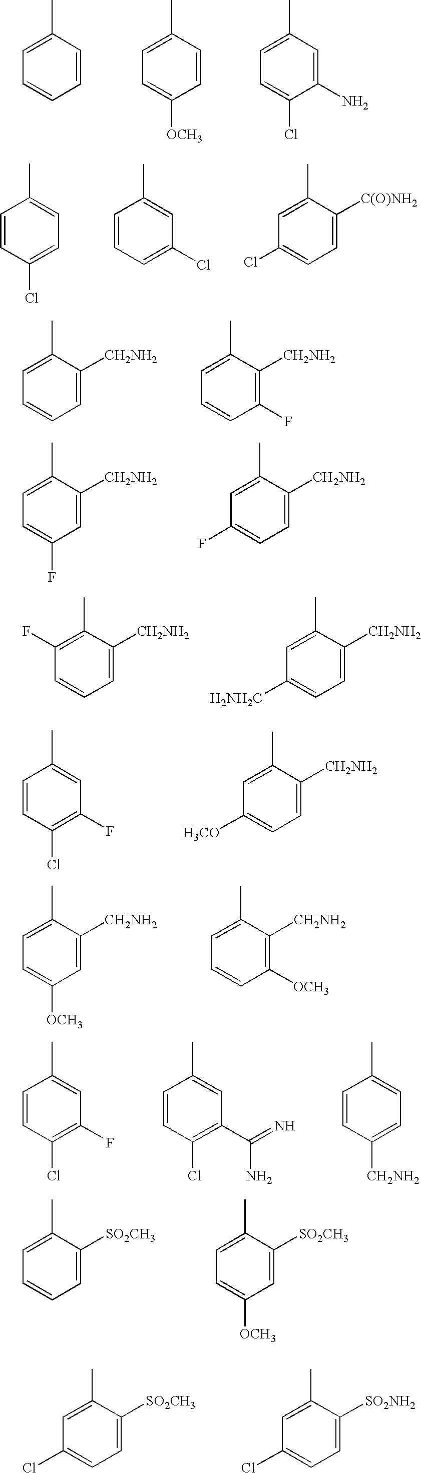 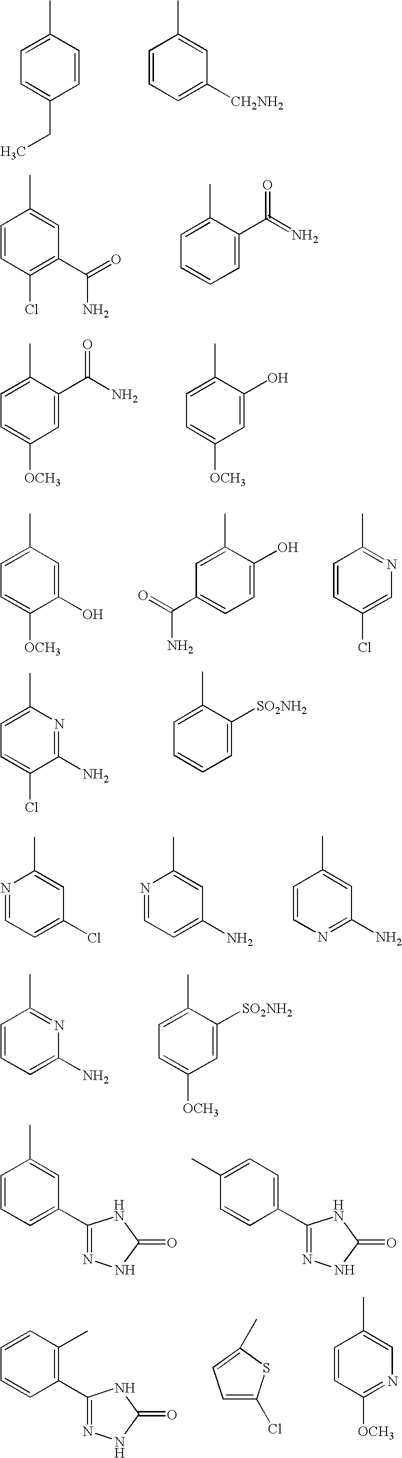 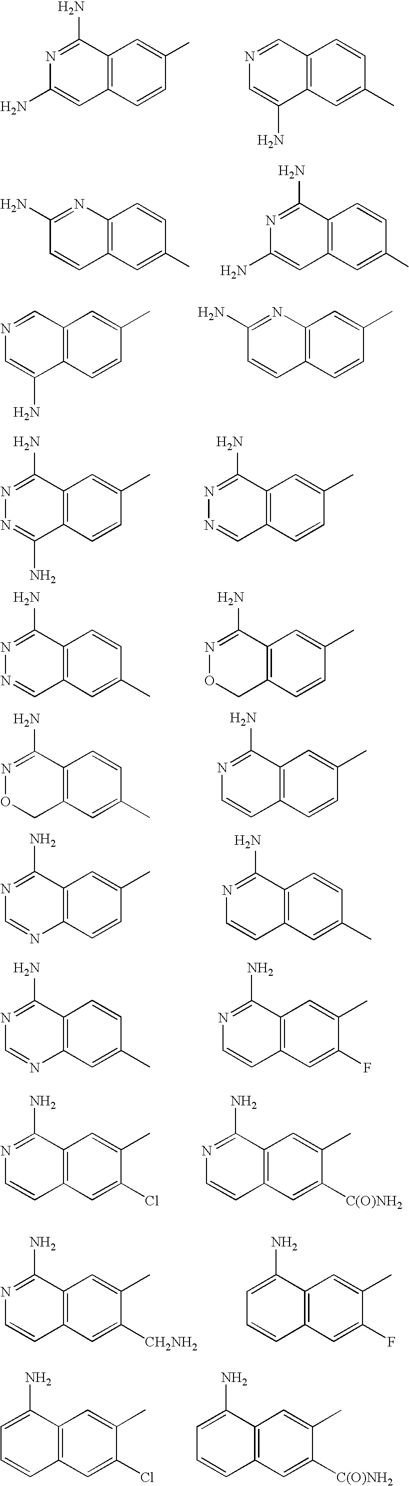 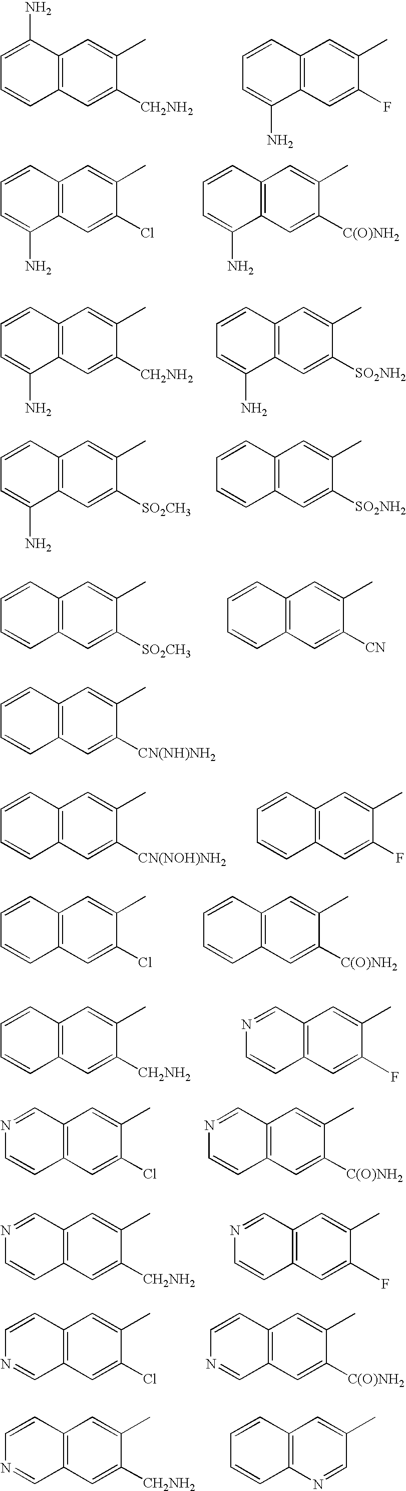 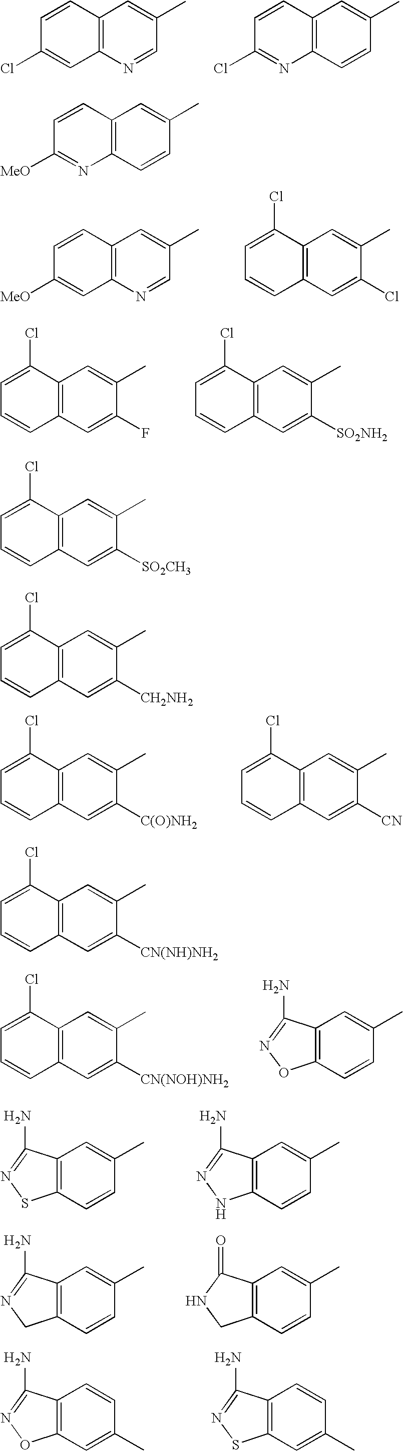 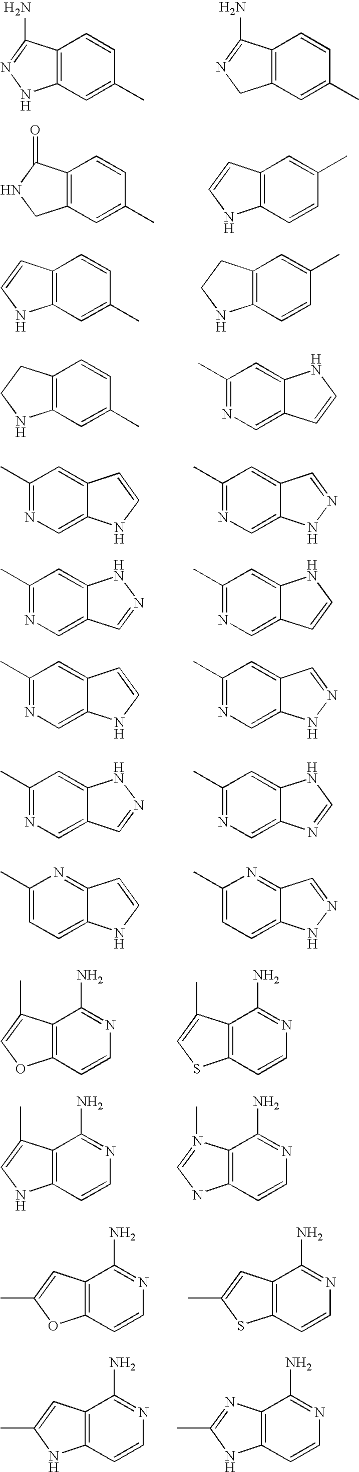 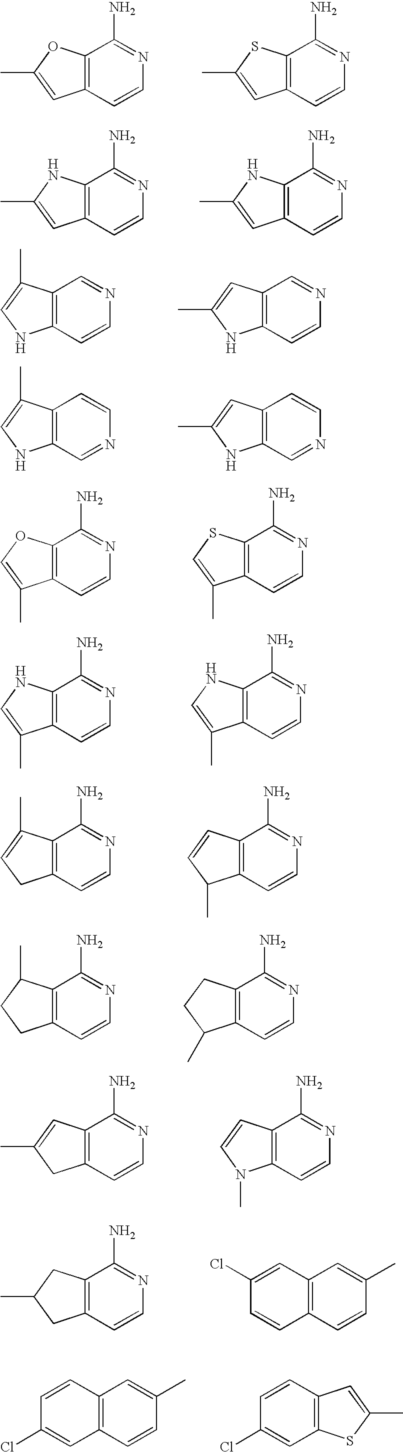 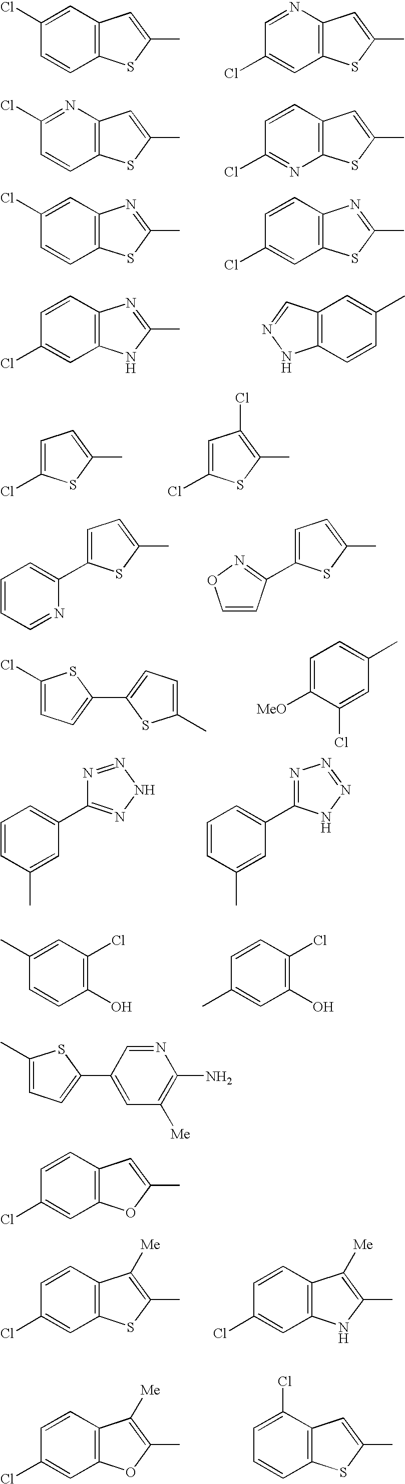 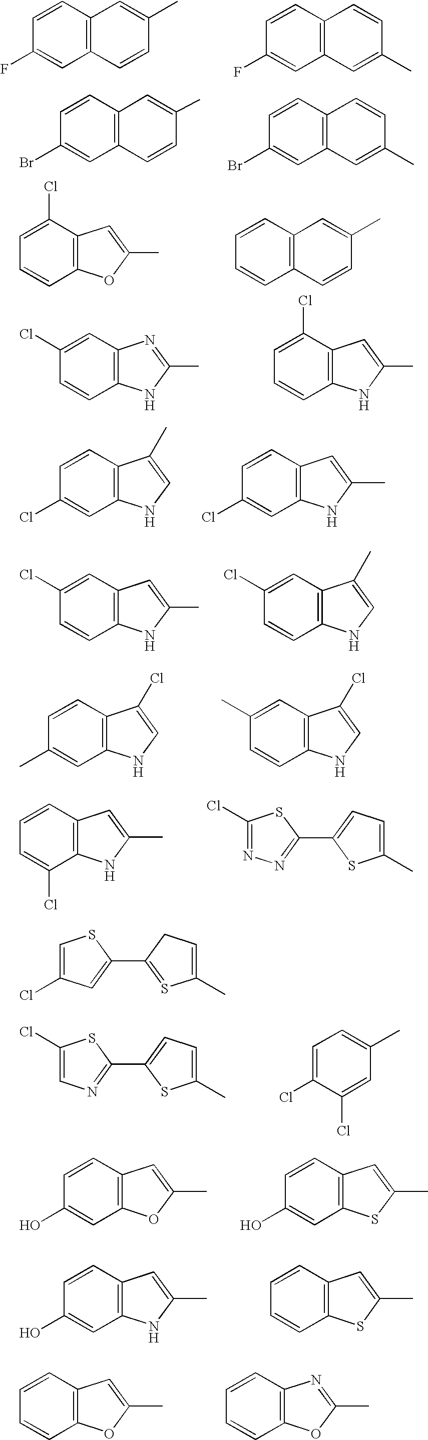 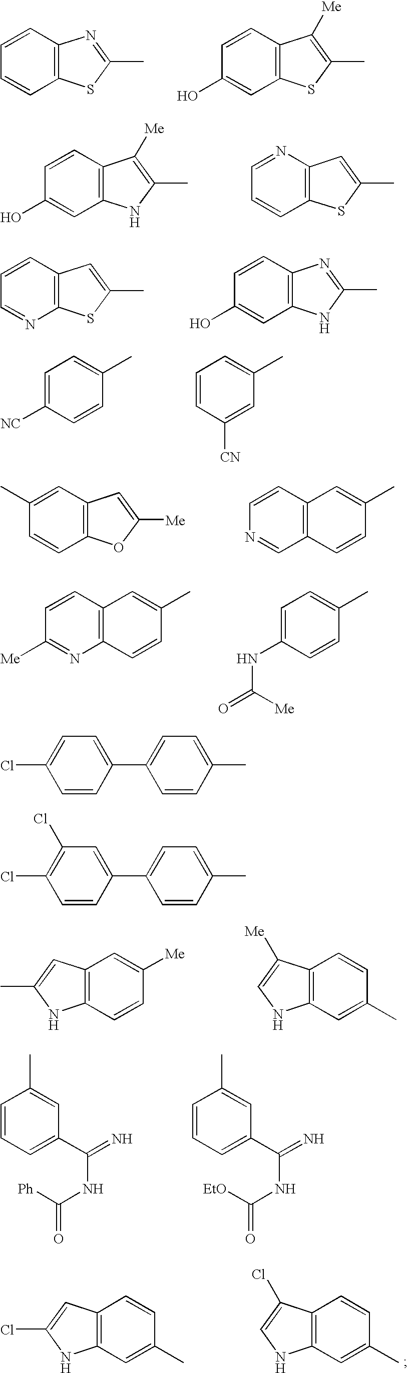
G₁ is selected from ═O or a 2-4 membered linear chain consisting of: 0-4 carbon atoms, 0-1 carbonyl groups, and 0-2 heteroatoms selected from O, N, and S(O)ₚ, and G₁ is substituted with 0-4 R³, and there are 0-1 double bonds; provided that other than an S—S, S—O, or O—O bond is present in G₁;
Z is selected from (CR³R^{3e})_{q}C(O)(CR³R^{3e})_{q1}, (CR³R^{3e})_{q}NR^{3b}(CR³R^{3e})_{q1}, (CR³R^{3e})_{q}C(O)NR^{3b}(CR³R^{3e})_{q1}, (CR³R^{3e})_{q}NR^{3b}C(O)(CR³R^{3e})_{q1}, (CR³R^{3e})_{q}NR^{3b}C(O)NR^{3b}(CR³R^{3e})_{q1}, (CR³R^{3e})_{q}S(O)₂(CR³R^{3e})_{q1}, (CR³R^{3e})_{q}SO₂NR^{3b}(CR³R^{3e})_{q1}, (CR³R^{3e})_{q}NR^{3b}SO₂(CR³R^{3e})_{q1}, wherein q+q1 total 1 or 2, provided that Z does not form a N—S, NCH₂N, NCH₂O, or NCH₂S bond with either group to which it is attached;
A is selected from one of the following carbocycles and heterocycles which are substituted with 0-2 R⁴; cyclohexyl, phenyl, piperidinyl, piperazinyl, pyridyl, pyrimidyl, furanyl, morpholinyl, thienyl, pyrrolyl, pyrrolidinyl, oxazolyl, isoxazolyl, thiazolyl, isothiazolyl, pyrazolyl, imidazolyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, 1,2,3-triazolyl, 1,2,4-triazolyl, 1,2,5-triazolyl, 1,3,4-triazolyl, benzofuranyl, benzothiofuranyl, indolinyl, indolyl, benzimidazolyl, benzoxazolyl, benzthiazolyl, indazolyl, benzisoxazolyl, benzisothiazolyl, and isoindazolyl;
X is selected from —CR²R^{2a})₁₋₂—, —C(O)—, —S(O)₂—, —NR²S(O)₂—, —NR²S(O)₂—, —NR²C(O)—, —C(O)NR²—, NR², —NR²CR²R^{2a}—, —CR²R^{2a}NR²—, O, —OCR²R^{2a}—, and —CR²R^{2a}O—;
R^{1a}, at each occurrence, is selected from H, R^{1b}, CH(CH₃)R^{1b}, C(CH₃)₂R^{1b}, CH₂R^{1b}, and CH₂CH₂R^{1b}, provided that R^{1a} forms other than an N-halo, N—S, or N—CN bond;
provided that R^{1a} is other than a substituted or unsubstituted 3,4-dihydroxyphenyl group;
alternatively, when two R^{1a} groups are attached to adjacent atoms or to the same carbon atom, together with the atoms to which they are attached, they form a 5-6 membered ring consisting of: carbon atoms and 0-2 heteroatoms selected from the group consisting of N, O, and S(O)ₚ, this ring being substituted with 0-2 R^{4b} and comprising: 0-3 ring double bonds;
R^{1b} is selected from H, CH₃, CH₂CH₃, F, Cl, Br, —CN, CF₃, OR², NR²R^{2a}, C(O)R^{2b}, CO₂R^{2b}, OC(O)R², CO₂R^{2a}, S(O)ₚR², NR²(CH₂)ᵣOR², NR²C(O)R^{2b}, C(O)NR²R^{2a}, SO₂NR²R^{2a}, NR²SO₂R², phenyl substituted with 0-2 R^{4b}, and 5-6 membered aromatic heterocycle consisting of carbon atoms and from 1-4 heteroatoms selected from the group consisting of N, O, and S(O)ₚ and substituted with 0-2 R^{4b}, provided that R^{1b} forms other than an O—O, N-halo, N—S, or N—CN bond;
R², at each occurrence, is selected from H, CF₃, CH₃, CH₂CH₃, CH₂CH₂CH₃, CH(CH₃)₂, phenyl substituted with 0-2 R^{4b}, a benzyl substituted with 0-2 R^{4b}, and 5-6 membered aromatic heterocycle substituted with 0-2 R^{4b} and consisting of: carbon atoms and 1-4 heteroatoms selected from the group consisting of N, O, and S(O)ₚ;
R^{2a}, at each occurrence, is selected from H, CF₃, CH₃, CH₂CH₃, CH₂CH₂CH₃, CH(CH₃)₂, benzyl, phenyl substituted with 0-2 R^{4b}, and 5-6 membered aromatic heterocycle substituted with 0-2 R^{4b} and consisting of: carbon atoms and 1-4 heteroatoms selected from the group consisting of N, O, and S(O)ₚ;
R^{2b}, at each occurrence, is selected from CF₃, C₁₋₄ alkoxy, CH₃, CH₂CH₃, CH₂CH₂CH₃, CH(CH₃)₂, benzyl, phenyl substituted with 0-2 R^{4b}, and 5-6 membered aromatic heterocycle substituted with 0-2 R^{4b} and consisting of: carbon atoms and 1-4 heteroatoms selected from the group consisting of N, O, and S(O)ₚ;
R^{2c}, at each occurrence, is selected from CF₃, OH, OCH₃, OCH₂CH₃, OCH₂CH₂CH₃, OCH(CH₃)₂, CH₃, CH₂CH₃, CH₂CH₂CH₃, CH(CH₃)₂, benzyl, phenyl substituted with 0-2 R^{4b}, and 5-6 membered aromatic heterocycle substituted with 0-2 R^{4b} and consisting of carbon atoms and from 1-4 heteroatoms selected from the group consisting of N, O, and S(O)ₚ;
alternatively, NR²R^{2a} forms a 5 or 6 membered saturated, partially saturated or unsaturated ring substituted with 0-2 R^{4b} and consisting of: 0-1 additional heteroatoms selected from the group consisting of N, O, and S(O)ₚ;
R⁴, at each occurrence, is selected from H, (CH₂)₂OR², CH₂OR², OR², F, Cl, Br, I, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —CN, NO₂, NR²R^{2a}, CH₂NR²R^{2a}, (CH₂)₂NR²R^{2a}, C(O)R^{2c}, NR²C(O)R^{2b}, C(O)NR²R^{2a}, SO₂NR²R^{2a}, CF₃, and CF₂CF₃;
R^{4a}, at each occurrence, is selected from H, ═O, (CH₂)ᵣOR², (CH₂)ᵣ—F, (CH₂)ᵣ—Br, (CH₂)ᵣCl, C₁₋₄ alkyl, (CH₂)ᵣ—CN, (CH₂)ᵣNO₂, (CH₂)ᵣNR²R^{2a}, (CH₂)ᵣC(O)R^{2c}, (CH₂)ᵣNR²C(O)R^{2b}, (CH₂)ᵣC(O)NR²R^{2a}, (CH₂)ᵣSO₂NR²R^{2a}, (CH₂)ᵣNR²SO₂NR²R^{2a}, (CH₂)ᵣNR²SO₂—C₁₋₄ alkyl, (CH₂)ᵣC(O)NHSO₂—C₁₋₄ alkyl, (CH₂)ᵣNR²SO₂R⁵, (CH₂)ᵣS(O)ₚR⁵, (CH₂)ᵣ(CF₂)ᵣCF₃, phenyl substituted with 0-1 R⁵, and a 5 membered aromatic heterocycle consisting of: carbon atoms and 1-3 heteroatoms selected from the group consisting of N, O, and S(O)ₚ substituted with 0-1 R⁵;
R^{4b}, at each occurrence, is selected from H, ═O, OR³, CH₂OR³, F, Cl, CH₃, CH₂CH₃, CH₂CH₂CH₃, CH(CH₃)₂, —CN, NO₂, NR³R^{3a}, CH₂NR³R^{3a}, C(O)R³, CH₂—C(O)R³, C(O)OR^{3c}, CH₂—C(O)OR^{3c}, NR³C(O)R^{3a}, CH₂NR³C(O)R^{3a}, C(O)NR³R^{3a}, CH₂—C(O)NR³R^{3a}, SO₂NR³R^{3a}, CH₂SO₂NR³R^{3a}, NR³SO₂—C₁₋₄ alkyl, CH₂NR³SO₂—C₁₋₄ alkyl, NR³SO₂-phenyl, CH₂NR³ SO₂-phenyl, S(O)ₚCF₃, CH₂S(O)ₚCF₃, S(O)ₚ—C₁₋₄ alkyl, CH₂S(O)ₚ—C₁₋₄ alkyl, S(O)ₚ-phenyl, CH₂S(O)ₚ-phenyl, and CF₃;
R⁵, at each occurrence, is selected from H, ═O, CH₃, CH₂CH₃, CH₂CH₂CH₃, CH(CH₃)₂, OR³, CH₂OR³, F, Cl, —CN, NO₂, NR³R^{3a}, CH₂NR³R^{3a}, C(O)R³, CH₂C(O)R³, C(O)OR^{3c}, CH₂C(O)OR^{3c}, NR³C(O)R^{3a}, C(O)NR³R^{3a}, SO₂NR³R^{3a}, NR³SO₂—C₁₋₄ alkyl, NR³SO₂CF₃, NR³SO₂-phenyl, S(O)ₚCF₃, S(O)ₚ—C₁₋₄ alkyl, S(O)ₚ-phenyl, CF₃, phenyl substituted with 0-2 R⁶, naphthyl substituted with 0-2 R⁶, and benzyl substituted with 0-2 R⁶;
R⁶, at each occurrence, is selected from H, OH, OR², F, Cl, CH₃, CH₂CH₃, CH₂CH₂CH₃, CH(CH₃)₂, —CN, NO₂, NR²R^{2a}, CH₂NR²R^{2a}, C(O)R^{2b}, CH₂C(O)R^{2b}, NR²C(O)R^{2b}, SO₂NR²R^{2a}, and NR²SO₂C₁₋₄ alkyl; and
r, at each occurrence, is selected from 0, 1, and 2.

In a fourth embodiment, the present invention provides a novel compound, wherein:
G is selected from the group: 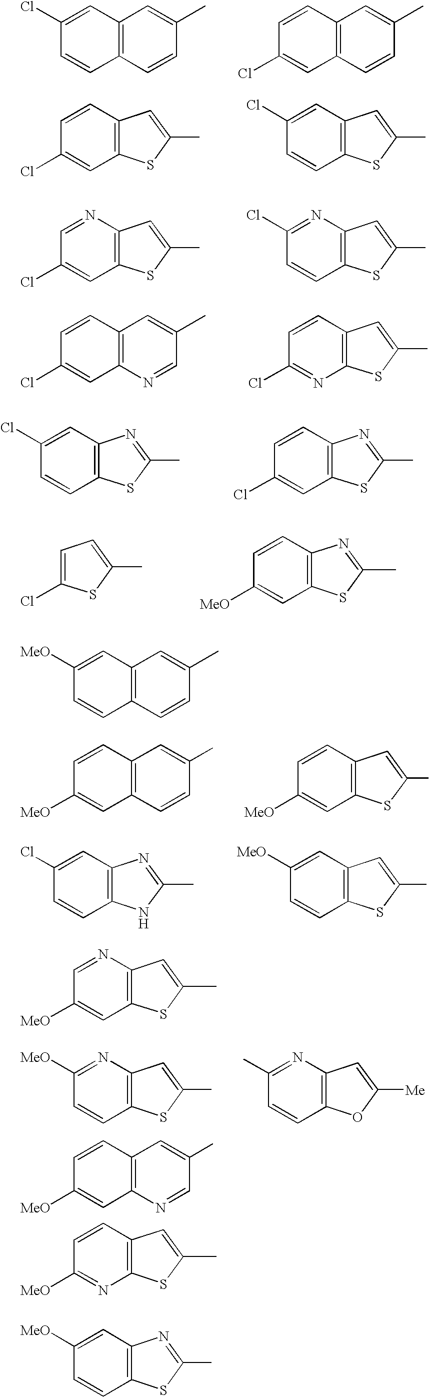 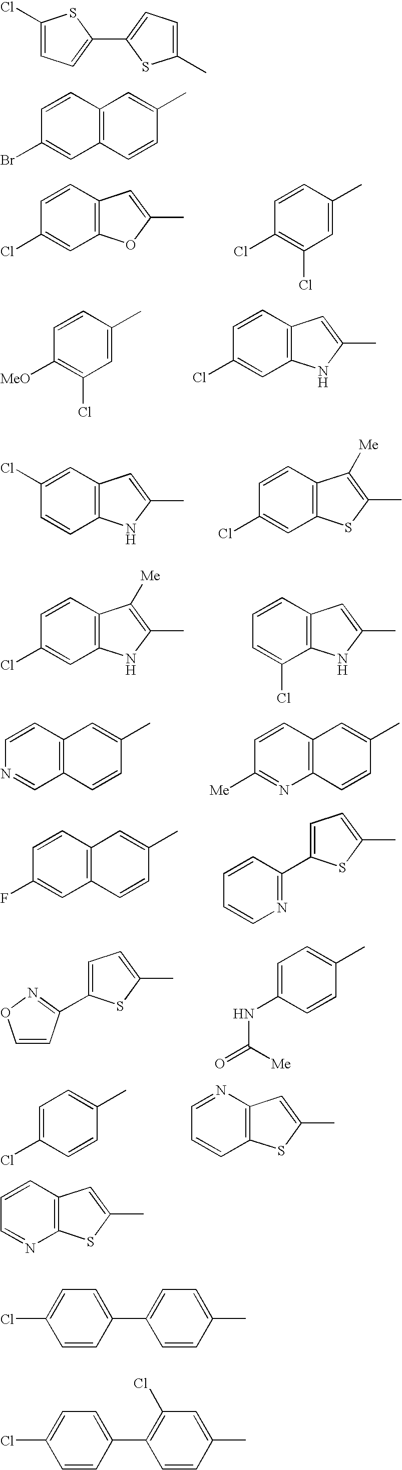 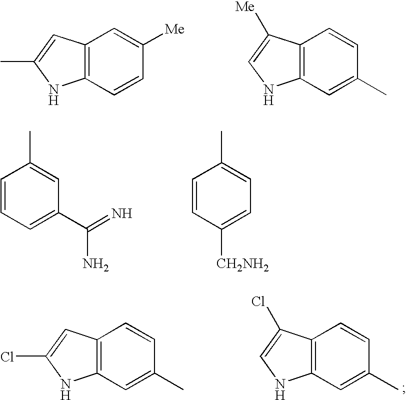
A is selected from cyclohexyl, indolinyl, piperidinyl, phenyl, pyridyl, and pyrimidyl, and is substituted with 0-2 R⁴;
X is selected from CH₂, C(O), —S(O)₂—, —NHC(O)—, —C(O)NH—, —CH₂NH—, O, and —CH₂O—;
Z is selected from C(O), CH₂NH, NHCH₂, C(O)NH, NHC(O), NHC(O)NH, S(O)₂, SO₂NH, and NHSO₂, wherein the left side of Z is attached to ring M, provided that Z does not form a N—S, NCH₂N, NCH₂O, or NCH₂S bond with either group to which it is attached;
R^{1a}, at each occurrence, is selected from H, R^{1b}, CH(CH₃)R^{1b}, C(CH₃)₂R^{1b}, and CH₂R^{1b}, provided that R^{1a} forms other than an N-halo, N—S, or N—CN bond;
R^{1b} is selected from CH₃, CH₂CH₃, F, Cl, Br, —CN, CF₃, OR², NR²R^{2a}, C(O)R^{2b}, CO₂R^{2b}, CO₂R^{2a}, S(O)ₚR², C(O)NR²R^{2a}, SO₂NR²R^{2a}, NR²SO₂R², and 5-6 membered aromatic heterocycle consisting of carbon atoms and from 1-4 heteroatoms selected from the group consisting of N, O, and S(O)ₚ and substituted with 0-2 R^{4b}, provided that R^{1b} forms other than an O—O, N-halo, N—S, or N—CN bond;
R², at each occurrence, is selected from H, CH₃, CH₂CH₃, CH₂CH₂CH₃, CH(CH₃)₂, phenyl substituted with 0-1 R^{4b}, benzyl substituted with 0-1 R^{4b}, and 5-6 membered aromatic heterocycle substituted with 0-1 R^{4b} and consisting of: carbon atoms and 1-4 heteroatoms selected from the group consisting of N, O, and S(O)ₚ;
R^{2a}, at each occurrence, is selected from H, CH₃, CH₂CH₃, CH₂CH₂CH₃, CH(CH₃)₂, benzyl, phenyl substituted with 0-1 R^{4b}, and 5-6 membered aromatic heterocycle substituted with 0-1 R^{4b} and consisting of: carbon atoms and 1-4 heteroatoms selected from the group consisting of N, O, and S(O)ₚ;
alternatively, NR²R^{2a} forms a 5 or 6 membered saturated, partially saturated or unsaturated ring substituted with 0-1 R^{4b} and consisting of: 0-1 additional heteroatoms selected from the group consisting of N, O, and S(O)ₚ;
R^{2b}, at each occurrence, is selected from OH, OCH₃, OCH₂CH₃, OCH₂CH₂CH₃, OCH(CH₃)₂, CH₃, CH₂CH₃, CH₂CH₂CH₃, CH(CH₃)₂, benzyl, phenyl substituted with 0-1 R^{4b}, and 5-6 membered aromatic heterocycle substituted with 0-1 R^{4b} and consisting of: carbon atoms and 1-4 heteroatoms selected from the group consisting of N, O, and S(O)ₚ;
R^{2c}, at each occurrence, is selected from OH, OCH₃, OCH₂CH₃, OCH₂CH₂CH₃, OCH(CH₃)₂, CH₃, CH₂CH₃, CH₂CH₂CH₃, CH(CH₃)₂, benzyl, phenyl substituted with 0-1 R^{4b}, and 5-6 membered aromatic heterocycle substituted with 0-1 R^{4b} and consisting of carbon atoms and from 1-4 heteroatoms selected from the group consisting of N, O, and S(O)ₚ;
R⁴, at each occurrence, is selected from OH, OR², CH₂OR², (CH₂)₂OR², F, Br, Cl, I, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, NR²R^{2a}, CH₂NR²R^{2a}, (CH₂)₂NR²R^{2a}, CF₃, and CF₂CF₃;
R^{4a}, at each occurrence, is selected from H, ═O, (CH₂)ᵣOR², F, Br, Cl, C₁₋₄ alkyl, (CH₂)ᵣNR²R^{2a}, (CH₂)ᵣC(O)R^{2c}, (CH₂)ᵣNR²C(O)R^{2b}, (CH₂)ᵣC(O)NR²R^{2a}, (CH₂)ᵣSO₂NR²R^{2a}, (CH₂)ᵣNR²SO₂R⁵, (CH₂)ᵣS(O)ₚR⁵, (CH₂)ᵣ(CF₂)ᵣCF₃, phenyl substituted with 0-1 R⁵, and a 5 membered aromatic heterocycle consisting of: carbon atoms and 1-3 N and is substituted with 1 R⁵;
R^{4b}, at each occurrence, is selected from H, ═O, OR³, CH₂OR³, F, Cl, CH₃, CH₂CH₃, CH₂CH₂CH₃, CH(CH₃)₂, —CN, NO₂, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂—C₁₋₄ alkyl, NR³SO₂-phenyl, S(O)ₚ—C₁₋₄ alkyl, S(O)ₚ-phenyl, and CF₃;
R⁵, at each occurrence, is selected from H, ═O, CH₃, CH₂CH₃, CH₂CH₂CH₃, CH(CH₃)₂, OR³, CH₂OR³, F, Cl, —CN, NO₂, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂—C₁₋₄ alkyl, NR³SO₂-phenyl, S(O)ₚ—C₁₋₄ alkyl, S(O)ₚ-phenyl, CF₃, phenyl substituted with 0-2 R⁶, naphthyl substituted with 0-2 R⁶, and benzyl substituted with 0-2 R⁶; and
R⁶, at each occurrence, is selected from H, OH, OR², F, Cl, CH₃, CH₂CH₃, CH₂CH₂CH₃, CH(CH₃)₂, —CN, NO₂, NR²R^{2a}, CH₂NR²R^{2a}, C(O)R^{2b}, CH₂C(O)R^{2b}, NR²C(O)R^{2b}, and SO₂NR²R^{2a}.

In a fifth embodiment, the present invention provides a novel compound, wherein:
P₁ is G₁-G;
M₁ is -Z-A-B;
G is selected from:

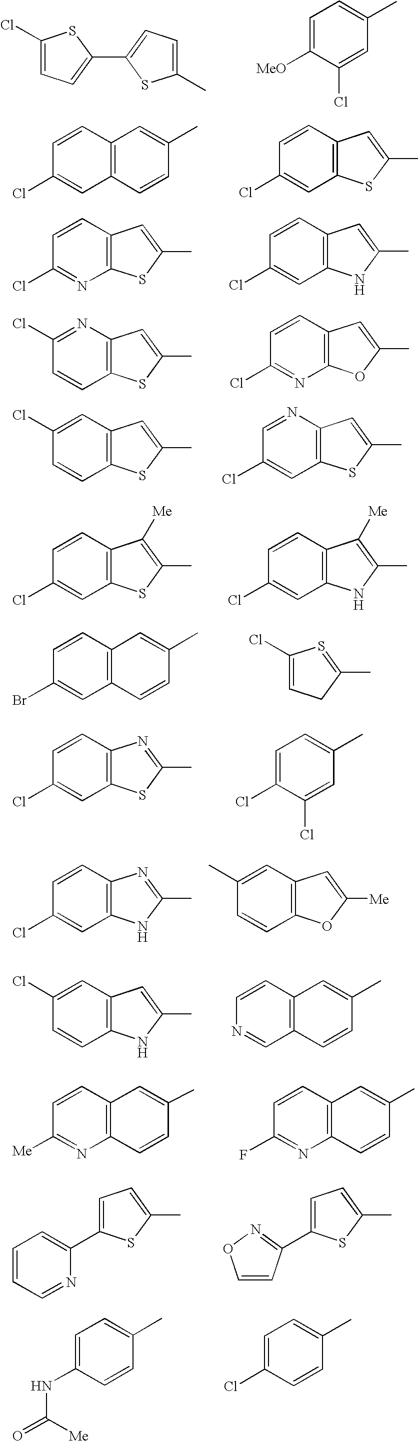

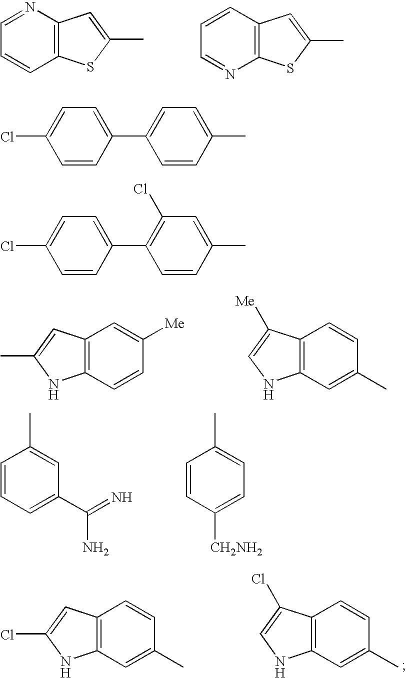

A is selected from the group: cyclohexyl, indolinyl, piperidinyl, phenyl, 2-pyridyl, 3-pyridyl, 2-pyrimidyl, 2-Cl-phenyl, 3-Cl-phenyl, 2-F-phenyl, 3-F-phenyl, 2-methylphenyl, 2-aminophenyl, and 2-methoxyphenyl;
B is selected from 2-oxo-pyridyl, 2-oxo-piperdinyl, 3-oxo-morpholinyl, phenyl, pyrrolidinyl, N-pyrrolidino-carbonyl, morpholinyl, N-morpholino-carbonyl, 1,2,3-triazolyl, imidazolyl, and benzimidazolyl, and is substituted with 0-1 R^{4a};
Z is selected from C(O), C(O)NH, NHC(O), NHC(O)NH, S(O)₂, SO₂NH, and NHSO₂, wherein the left side of Z is attached to ring M, provided that Z does not form a N—S, NCH₂N, NCH₂O, or NCH₂S bond with either group to which it is attached;
R^{1a}, at each occurrence, is selected from H, CH₃, CH₂CH₃, CH₂CH₂CH₃, CH₂F, CH₂Cl, Br, CH₂Br, —CN, CH₂CN, CF₃, CH₂CF₃, OCH₃, CH₂OH, C(CH₃)₂OH, CH₂OCH₃, NH₂, CH₂NH₂, NHCH₃, CH₂NHCH₃, N(CH₃)₂, CH₂N(CH₃)₂, CO₂H, COCH₃, CO₂CH₃, CH₂CO₂CH₃, SCH₃, CH₂SCH₃, S(O)CH₃, CH₂S(O)CH₃, S(O)₂CH₃, CH₂S(O)₂CH₃, C(O)NH₂, CH₂C(O)NH₂, SO₂NH₂, CH₂SO₂NH₂, NHSO₂CH₃, CH₂NHSO₂CH₃, pyridin-2-yl, pyridin-3-yl, pyridin-4-yl, pyridin-2-yl-N-oxide, pyridin-3-yl-N-oxide, pyridin-4-yl-N-oxide, imidazol-1-yl, CH₂-imidazol-1-yl, 4-methyl-oxazol-2-yl, 4-N,N-dimethylaminomethyl-oxazol-2-yl, 1,2,3,4-tetrazol-1-yl, 1,2,3,4-tetrazol-5-yl, CH₂-1,2,3,4-tetrazol-1-yl, and CH₂-1,2,3,4-tetrazol-5-yl, provided that R^{1a} forms other than an N-halo, N—S, or N—CN bond;
R², at each occurrence, is selected from H, CH₃, CH₂CH₃, CH₂CH₂CH₃, CH(CH₃)₂, phenyl substituted with 0-1 R^{4b}, benzyl substituted with 0-1 R^{4b}, and 5 membered aromatic heterocycle substituted with 0-1 R^{4b} and consisting of: carbon atoms and 1-4 heteroatoms selected from the group consisting of N, O, and S(O)ₚ;
R^{2a}, at each occurrence, is selected from H, CH₃, and CH₂CH₃;
alternatively, NR²R^{2a} forms a 5 or 6 membered saturated, partially saturated or unsaturated ring substituted with 0-1 R^{4b} and consisting of: 0-1 additional heteroatoms selected from the group consisting of N, O, and S(O)ₚ;
R^{2b}, at each occurrence, is selected from OH, OCH₃, OCH₂CH₃, CH₃, and CH₂CH₃;
R^{2c}, at each occurrence, is selected from OH, OCH₃, OCH₂CH₃, CH₃, and CH₂CH₃;
R^{4a} is selected from C₁₋₄ alkyl, CF₃, OR², CH₂OR², (CH₂)₂OR², NR²R^{2a}, CH₂NR²R^{2a}, (CH₂)₂NR²R^{2a}, S(O)ₚR⁵, SO₂NR²R^{2a}, and 1-CF₃-tetrazol-2-yl;
R^{4b}, at each occurrence, is selected from H, ═O, OR³, CH₂OR³, F, Cl, CH₃, CH₂CH₃, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂-phenyl, S(O)₂CH₃, S(O)₂-phenyl, and CF₃;
R⁵, at each occurrence, is selected from H, ═O, CH₃, CH₂CH₃, OR³, CH₂OR³, F, Cl, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂—C₁₋₄ alkyl, NR³SO₂-phenyl, S(O)₂—CH₃, S(O)₂-phenyl, CF₃, phenyl substituted with 0-2 R⁶, naphthyl substituted with 0-2 R⁶, and benzyl substituted with 0-2 R⁶; and
R⁶, at each occurrence, is selected from H, OH, OR², F, Cl, CH₃, CH₂CH₃, NR²R^{2a}, CH₂NR²R^{2a}, C(O)R^{2b}, CH₂C(O)R^{2b}, NR²C(O)R^{2b}, and SO₂NR²R^{2a}.

In a sixth embodiment, the present invention provides a novel compound, wherein:

A is selected from the group: phenyl, 2-pyridyl, 3-pyridyl, 2-pyrimidyl, 2-Cl-phenyl, 3-Cl-phenyl, 2-F-phenyl, 3-F-phenyl, 2-methylphenyl, 2-aminophenyl, and 2-methoxyphenyl;
B is selected from the group: 2-oxo-pyridyl, 2-oxo-piperdinyl, 2-(aminosulfonyl)phenyl, 2-(methylaminosulfonyl)phenyl, N-pyrrolidino-carbonyl, 2-(methylsulfonyl)phenyl, 2-(N,N-dimethylaminomethyl)phenyl, 2-(N-methylaminomethyl)phenyl, 2-(N-ethyl-N-methylaminomethyl)phenyl, 2-(N-pyrrolidinylmethyl)phenyl, 1-methyl-2-imidazolyl, 2-methyl-1-imidazolyl, 2-(dimethylaminomethyl)-1-imidazolyl, 2-(methylaminomethyl)-1-imidazolyl, 2-(N-(cyclopropylmethyl)aminomethyl)phenyl, 2-(N-(cyclobutyl)aminomethyl)phenyl, 2-(N-(cyclopentyl)aminomethyl)phenyl, 2-(N-(4-hydroxypiperidinyl)methyl)phenyl, and 2-(N-(3-hydroxypyrrolidinyl)methyl)phenyl; and
G₁ is selected from: ═O, (CHR³)NHSO₂, (CHR³)NHC(O), (CHR³)NHC(O)NH, (CHR³)(CHR³)NHSO₂, (CHR³)(CHR³)NHC(O), (CHR³)SO₂, (CHR³)(CHR³)SO₂, (CR³R³)SO₂NH, (CHR³)(CHR³)SO₂NH, (CR³R³)C(O)NH, (CHR³)(CHR³)C(O)NH, NHC(O)NH, NHSO₂, NHSO₂CH₂, CH₂O, OCH₂, CH₂CH₂O, CH₂NH, CH₂CH₂NH, CH₂NHCH₂, NHCH₂, NHCH₂CH₂, CH₂C(O)NHSO₂, and CH₂SO₂NHC(O).

In a seventh embodiment, the present invention provides a novel compound of formula IIa or IIb: 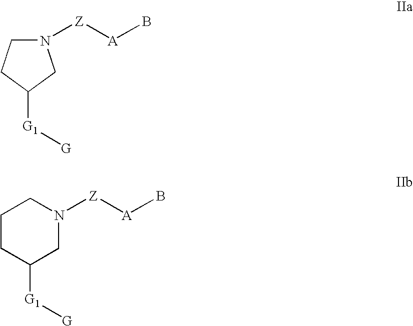
wherein: G₁ is selected from: ═O, CH₂NHSO₂, CH₂NHC(O), CH₂NHC(O)NH, CH₂CH₂NHSO₂, CH₂CH₂NHC(O), CH₂SO₂, CH₂CH₂SO₂, CH₂SO₂NH, CH₂CH₂SO₂NH, CH₂C(O)NH, CH₂CH₂C(O)NH, NHC(O)NH, NHSO₂, NHSO₂CH₂, CH₂O, OCH₂, CH₂CH₂O, CH₂NH, CH₂CH₂NH, CH₂NHCH₂, NHCH₂, NHCH₂CH₂, CH₂C(O)NHSO₂, and CH₂SO₂NHC(O).

In an eight embodiment, the present invention provides a novel compound, wherein the compound is selected from Examples 1-70 or a pharmaceutically acceptable salt form thereof.

In another embodiment, the present invention provides a novel pharmaceutical composition, comprising: a pharmaceutically acceptable carrier and a therapeutically effective amount of a compound of the present invention or a pharmaceutically acceptable salt form thereof.

In another embodiment, the present invention provides a novel method for treating a thromboembolic disorder, comprising: administering to a patient in need thereof a therapeutically effective amount of a compound of the present invention or a pharmaceutically acceptable salt form thereof.

In another preferred embodiment, the present invention provides a novel method, wherein the thromboembolic disorder is selected from the group consisting of arterial cardiovascular thromboembolic disorders, venous cardiovascular thromboembolic disorders, and thromboembolic disorders in the chambers of the heart.

In another preferred embodiment, the present invention provides a novel method, wherein the thromboembolic disorder is selected from unstable angina, an acute coronary syndrome, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.

In another embodiment, the present invention provides a novel method for treating a thromboembolic disorder, comprising: administering to a patient in need thereof a therapeutically effective amount of a first and second therapeutic agent, wherein the first therapeutic agent is compound of the present invention or a pharmaceutically acceptable salt thereof and the second therapeutic agent is at least one agent selected from a second factor Xa inhibitor, an anti-coagulant agent, an anti-platelet agent, a thrombin inhibiting agent, a thrombolytic agent, and a fibrinolytic agent.

In another preferred embodiment, the present invention provides a novel method, wherein the second therapeutic agent is at least one agent selected from warfarin, unfractionated heparin, low molecular weight heparin, synthetic pentasaccharide, hirudin, argatrobanas, aspirin, ibuprofen, naproxen, sulindac, indomethacin, mefenamate, droxicam, diclofenac, sulfinpyrazone, piroxicam, ticlopidine, clopidogrel, tirofiban, eptifibatide, abciximab, melagatran, disulfatohirudin, tissue plasminogen activator, modified tissue plasminogen activator, anistreplase, urokinase, and streptokinase.

In another preferred embodiment, the present invention provides a novel method, wherein the second therapeutic agent is at least one anti-platelet agent.

In another preferred embodiment, the present invention provides a novel method, wherein the anti-platelet agent is aspirin and clopidogrel.

In another preferred embodiment, the present invention provides a novel method, wherein the anti-platelet agent is clopidogrel.

In another embodiment, the present invention provides a novel article of manufacture, comprising:
(a) a first container;
(b) a pharmaceutical composition located within the first container, wherein the composition, comprises: a first therapeutic agent, comprising: a compound of the present invention or a pharmaceutically acceptable salt form thereof; and
(c) a package insert stating that the pharmaceutical composition can be used for the treatment of a thromboembolic disorder.

In another preferred embodiment, the present invention provides a novel article of manufacture, further comprising:
(d) a second container;
   wherein components (a) and (b) are located within the second container and component (c) is located within or outside of the second container.

In another embodiment, the present invention provides a novel article of manufacture, comprising:
(a) a first container;
(b) a pharmaceutical composition located within the first container, wherein the composition, comprises: a first therapeutic agent, comprising: a compound of the present invention or a pharmaceutically acceptable salt form thereof; and
(c) a package insert stating that the pharmaceutical composition can be used in combination with a second therapeutic agent to treat a thromboembolic disorder.

In another preferred embodiment, the present invention provides a novel article of manufacture, further comprising:
(d) a second container;
   wherein components (a) and (b) are located within the second container and component (c) is located within or outside of the second container.

In another embodiment, the present invention provides novel compounds as described above for use in therapy.

In another embodiment, the present invention provides the use of novel compounds as described above for the manufacture of a medicament for the treatment of a thromboembolic disorder.

The present invention may be embodied in other specific forms without departing from the spirit or essential attributes thereof. This invention encompasses all combinations of preferred aspects of the invention noted herein. It is understood that any and all embodiments of the present invention may be taken in conjunction with any other embodiment or embodiments to describe additional more preferred embodiments. It is also to be understood that each individual element of the preferred embodiments is intended to be taken individually as its own independent preferred embodiment. Furthermore, any element of an embodiment is meant to be combined with any and all other elements from any embodiment to describe an additional embodiment.

Definitions

The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated. All processes used to prepare compounds of the present invention and intermediates made therein are considered to be part of the present invention. All tautomers of shown or described compounds are also considered to be part of the present invention.

The term “linear chain,” as used herein to describe linker G₁, is intended to mean a series of atoms (i.e., carbon, oxygen, nitrogen, and sulfur) that are connected together one at a time to form a chain. Thus, a chain atom is connected to one other chain atom if it is a terminal atom or two other chain atoms if is non-terminal. None of these chain atoms are bonded together, directly or indirectly, through a ring. Examples of a 5-membered linear chain include C(O)NHCH₂NHC(O) and NHC(O)CH₂S(O)₂NH, but not 1-amino-2-carbamoyl-cyclohexane. The number of chain atoms is determined by counting each atom in the chain, but not any atom substituted thereon. Thus, the 3 oxygen atoms and 4 hydrogen atoms of the group S(O)₂NHCH₂NHC(O) are not counted, and S(O)₂NHCH₂NHC(O) is a 5-membered chain, not a 12-membered chain.

Preferably, the molecular weight of compounds of the present invention is less than about 500, 550, 600, 650, 700, 750, or 800 grams per mole. Preferably, the molecular weight is less than about 800 grams per mole. More preferably, the molecular weight is less than about 750 grams per mole. Even more preferably, the molecular weight is less than about 700 grams per mole.

The term “substituted,” as used herein, means that any one or more hydrogens on the designated atom is replaced with a selection from the indicated group, provided that the designated atom's normal valency is not exceeded, and that the substitution results in a stable compound. When a substituent is keto (i.e., ═O), then 2 hydrogens on the atom are replaced. Keto substituents are not present on aromatic moieties. Ring double bonds, as used herein, are double bonds that are formed between two adjacent ring atoms (e.g., C═C, C═N, or N═N). The present invention, in general, does not cover groups such as N-halo, S(O)H, and SO₂H.

The present invention is intended to include all isotopes of atoms occurring in the present compounds. Isotopes include those atoms having the same atomic number but different mass numbers. By way of general example and without limitation, isotopes of hydrogen include tritium and deuterium. Isotopes of carbon include C-13 and C-14.

When any variable (e.g., R⁶) occurs more than one time in any constituent or formula for a compound, its definition at each occurrence is independent of its definition at every other occurrence. Thus, for example, if a group is shown to be substituted with 0-2 R⁶, then said group may optionally be substituted with up to two R⁶ groups and R⁶ at each occurrence is selected independently from the definition of R⁶. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom via which such substituent is bonded to the rest of the compound of a given formula, then such substituent may be bonded via any atom in such substituent. Combinations of substituents and/or variables are permissible only if such combinations result in stable compounds.

In cases wherein there are amines on the compounds of this invention, these can be converted to amine N-oxides by treatment with an oxidizing agent (e.g., MCPBA and/or hydrogen peroxides) to afford other compounds of this invention. Thus, all shown and claimed amines are considered to cover both the shown amine and its N-oxide (N→O) derivative.

As used herein, “alkyl” is intended to include both branched and straight-chain saturated aliphatic hydrocarbon groups having the specified number of carbon atoms. C₁₋₆ alkyl, is intended to include C₁, C₂, C₃, C₄, C₅, and C₆ alkyl groups. Examples of alkyl include, but are not limited to, methyl, ethyl, n-propyl, i-propyl, n-butyl, s-butyl, t-butyl, n-pentyl, and s-pentyl. “Haloalkyl” is intended to include both branched and straight-chain saturated aliphatic hydrocarbon groups having the specified number of carbon atoms, substituted with 1 or more halogen (for example —CᵥF_{w} where v=1 to 3 and w=1 to (2v+1)). Examples of haloalkyl include, but are not limited to, trifluoromethyl, trichloromethyl, pentafluoroethyl, and pentachloroethyl. “Alkoxy” represents an alkyl group as defined above with the indicated number of carbon atoms attached through an oxygen bridge. C₁₋₆ alkoxy, is intended to include C₁, C₂, C₃, C₄, C₅, and C₆ alkoxy groups. Examples of alkoxy include, but are not limited to, methoxy, ethoxy, n-propoxy, i-propoxy, n-butoxy, s-butoxy, t-butoxy, n-pentoxy, and s-pentoxy. “Cycloalkyl” is intended to include saturated ring groups, such as cyclopropyl, cyclobutyl, or cyclopentyl. C₃₋₇ cycloalkyl is intended to include C₃, C₄, C₅, C₆, and C₇ cycloalkyl groups. Alkenyl” is intended to include hydrocarbon chains of either straight or branched configuration and one or more unsaturated carbon-carbon bonds that may occur in any stable point along the chain, such as ethenyl and propenyl. C₂₋₆ alkenyl is intended to include C₂, C₃, C₄, C₅, and C₆ alkenyl groups. “Alkynyl” is intended to include hydrocarbon chains of either straight or branched configuration and one or more triple carbon-carbon bonds that may occur in any stable point along the chain, such as ethynyl and propynyl. C₂₋₆ Alkynyl is intended to include C₂, C₃, C₄, C₅, and C₆ alkynyl groups.

“Halo” or “halogen” as used herein refers to fluoro, chloro, bromo, and iodo; and “counterion” is used to represent a small, negatively charged species such as chloride, bromide, hydroxide, acetate, and sulfate.

As used herein, “carbocycle” or “carbocyclic residue” is intended to mean any stable 3, 4, 5, 6, or 7-membered monocyclic or bicyclic or 7, 8, 9, 10, 11, 12, or 13-membered bicyclic or tricyclic ring, any of which may be saturated, partially unsaturated, or unsaturated (aromatic). Examples of such carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane, [2.2.2]bicyclooctane, fluorenyl, phenyl, naphthyl, indanyl, adamantyl, and tetrahydronaphthyl. As shown above, bridged rings are also included in the definition of carbocycle (e.g., [2.2.2]bicyclooctane). A bridged ring occurs when one or more carbon atoms link two non-adjacent carbon atoms. Preferred bridges are one or two carbon atoms. It is noted that a bridge always converts a monocyclic ring into a trycyclic ring. When a ring is bridged, the substituents recited for the ring may also be present on the bridge.

As used herein, the term “heterocycle” or “heterocyclic group” is intended to mean a stable 3, 4, 5, 6, or 7-membered monocyclic or 7, 8, 9, 10, 11, or 12-membered bicyclic or tricyclic heterocyclic ring which is saturated, partially unsaturated or unsaturated (aromatic), and which consists of carbon atoms and 1, 2, 3, 4, or 5 ring heteroatoms independently selected from the group consisting of N, O and S. Heterocycle includes any bicyclic group in which one heterocyclic ring is fused to a second ring, which may be carbocyclic (e.g. benzo fusion) or heterocyclic. When a heterocycle is referred to as an “aromatic heterocycle” or “heteroaryl,” this means that a fully unsaturated, i.e., aromatic, ring is present in the heterocycle. An aromatic heterocycle only requires one ring to be aromatic, if more than one ring is present. The aromatic portion of the aromatic heterocycle can be a carbocycle or heterocycle. The nitrogen and sulfur heteroatoms in the heterocycle may optionally be oxidized (i.e., N→O and S(O)ₚ). The nitrogen atom may be unsubstituted (i.e., N or NH) or substituted (i.e., NR wherein R is a substituent) and may optionally be quaternized. The heterocyclic ring may be attached to its pendant group at any heteroatom or carbon atom that results in a stable structure. The heterocyclic rings described herein may be substituted on a carbon or on a nitrogen atom, if the resulting compound is stable. It is preferred that when the total number of S and O atoms in the heterocycle exceeds 1, then these heteroatoms are not adjacent to one another. It is preferred that the total number of S and O atoms in the heterocycle is not more than 1. It is to be noted that total number of S and O atoms in the aromatic heterocycle is not more than 1. Bridged and spiro rings are also included in the definition of heterocycle. A bridged ring occurs when one or more atoms (i.e., C, O, N, or S) link two non-adjacent carbon or nitrogen atoms. Preferred bridges include, but are not limited to, one carbon atom, two carbon atoms, one nitrogen atom, two nitrogen atoms, and a carbon-nitrogen group. It is noted that a bridge always converts a monocyclic ring into a trycyclic ring. When a ring is bridged, the substituents recited for the ring may also be present on the bridge. Spiro rings are formed when to or more atoms (i.e., C, O, N, or S) of a chain are attached to the same carbon atom of a heterocycle (or carbocycle if fused to a heterocycle). When a spiro ring is present, the substituents recited for the ring may also be present on the spiro.

Examples of heterocycles include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocycles.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 1,2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. Lists of suitable salts are found in Remington's Pharmaceutical Sciences, 18th ed., Mack Publishing Company, Easton, Pa., 1990, p 1445, the disclosure of which is hereby incorporated by reference.

Since prodrugs are known to enhance numerous desirable qualities of pharmaceuticals (e.g., solubility, bioavailability, manufacturing, etc.) the compounds of the present invention may be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a mammalian subject. Prodrugs the present invention are prepared by modifying functional groups present in the compound in such a way that the modifications are cleaved, either in routine manipulation or in vivo, to the parent compound. Prodrugs include compounds of the present invention wherein a hydroxy, amino, or sulfhydryl group is bonded to any group that, when the prodrug of the present invention is administered to a mammalian subject, it cleaves to form a free hydroxyl, free amino, or free sulfhydryl group, respectively. Examples of prodrugs include, but are not limited to, acetate, formate, and benzoate derivatives of alcohol and amine functional groups in the compounds of the present invention.

“Stable compound” and “stable structure” are meant to indicate a compound that is sufficiently robust to survive isolation to a useful degree of purity from a reaction mixture, and formulation into an efficacious therapeutic agent. It is preferred that there presently recited compounds do not contain a N-halo, S(O)₂H, or S(O)H group.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. When a substituent is keto (i.e., ═O) group, then 2 hydrogens on the atom are replaced.

As used herein, “treating” or “treatment” cover the treatment of a disease-state in a mammal, particularly in a human, and include: (a) preventing the disease-state from occurring in a mammal, in particular, when such mammal is predisposed to the disease-state but has not yet been diagnosed as having it; (b) inhibiting the disease-state, i.e., arresting it development; and/or (c) relieving the disease-state, i.e., causing regression of the disease state.

“Therapeutically effective amount” is intended to include an amount of a compound of the present invention that is effective when administered alone or in combination to inhibit factor Xa. “Therapeutically effective amount” is also intended to include an amount of the combination of compounds claimed that is effective to inhibit factor Xa. The combination of compounds is preferably a synergistic combination. Synergy, as described, for example, by Chou and Talalay, Adv. Enzyme Regul. 1984, 22:27-55, occurs when the effect (in this case, inhibition of factor Xa) of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased antithrombotic effect, or some other beneficial effect of the combination compared with the individual components.

Synthesis

All references cited herein are hereby incorporated in their entirety herein by reference.

The compounds of the present invention can be prepared in a number of ways known to one skilled in the art of organic synthesis. The compounds of the present invention can be synthesized using the methods described below, together with synthetic methods known in the art of synthetic organic chemistry, or by variations thereon as appreciated by those skilled in the art. Preferred methods include, but are not limited to, those described below. The reactions are performed in a solvent appropriate to the reagents and materials employed and suitable for the transformations being effected. Those skilled in the art of organic synthesis understand that the functionality present on the molecule should be consistent with the transformations proposed. This will sometimes require a judgment to modify the order of the synthetic steps or to select one particular process scheme over another in order to obtain a desired compound of the invention.

Another major consideration in the planning of any synthetic route in this field is the judicious choice of the protecting group used for protection of reactive functional groups present in the compounds described in this invention. An authoritative account describing the many alternatives to the trained practitioner is Greene and Wuts (Protective Groups In Organic Synthesis, Wiley and Sons, 1991).

Compounds of the present invention may be prepared by the processes described in the following reaction schemes as well as by methods known to those skilled in the art. Exemplary reagents and procedures for these reactions are shown below and in the Examples.

As shown in Scheme 1, 1-benzyl-3-methylaminopyrrolidine (1) (J. Org Chem. 1961, 26, 4955), can be converted to 3 by protection followed by deprotection of benzyl group (via 2). The BOC protecting group may be used in place of the trifluoroacetyl group. Compound 3 can then be converted to 4 by acylation with an acid. Deprotection of trifluoroacetyl group and then sulfonylation with a sulfonyl halide can provide 6.

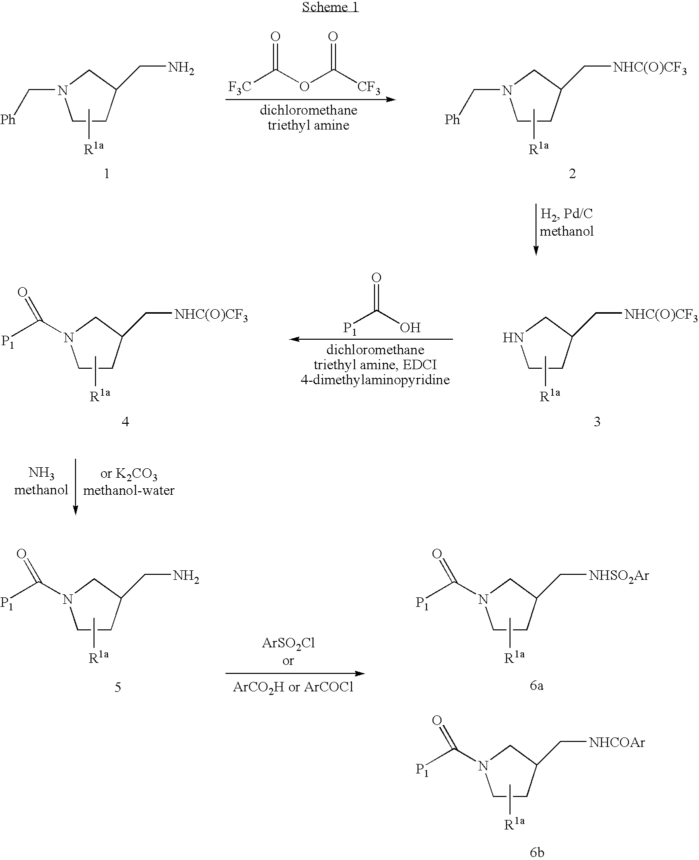

Alternatively, the amine 3 can react with a sulfonyl halide to afford 7 (see Scheme 2). Deprotection of the trifluoroacetyl group and acylation with an acid can provide 9.

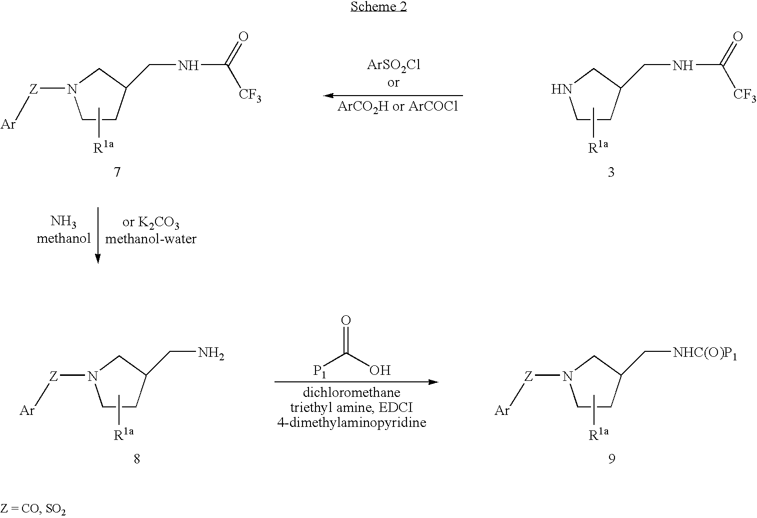

Amines of type 5 or 8 can also be acylated or sulfonylated to afford compounds with both amino groups of 3-aminomethylpyrrolidine being acylated or sulfonylated.

Compounds of the present invention can also be prepared as outlined in Scheme 3 and via standard methods known to those skilled in the art.

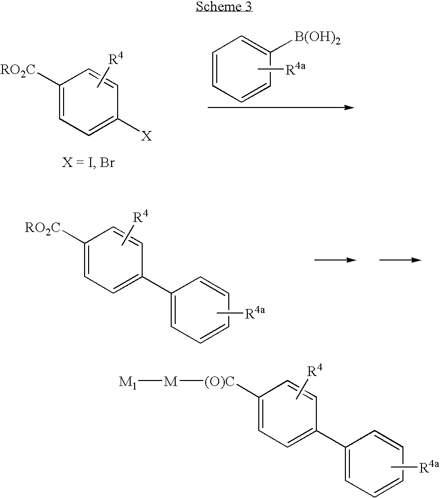

Compounds of the present invention can also be prepared via indole derivatives as outlined in Schemes 4. Following the standard methods known to those skilled in the art, the desired compounds can be obtained.

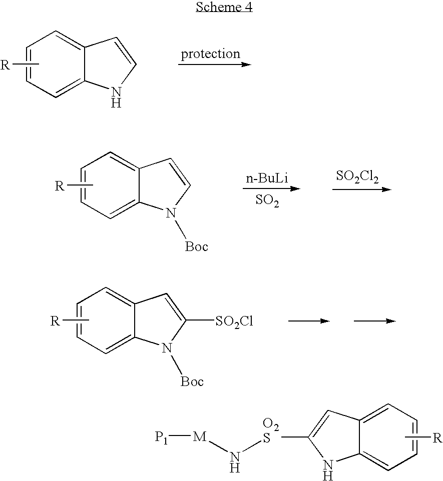

Alternatively, compounds of the present invention that are benzolthiophene derivatives can also be prepared as outlined in Scheme 5, and standard methods known to those skilled in the art.

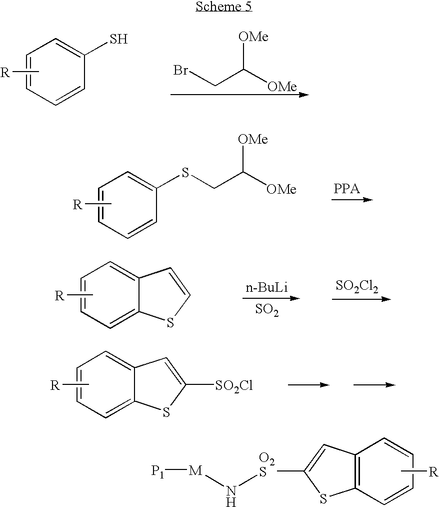

Compounds of present invention that are herteroaryl derivatives can also be prepared as outlined in Scheme 6, and standard methods known to those skilled in the art.

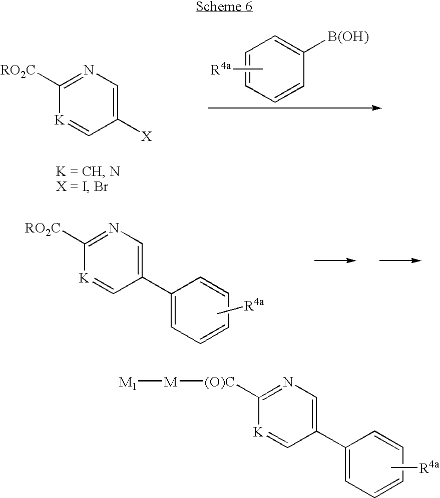

Additional compounds of the present invention can also be synthesized as outlined in Scheme 7, and standard methods known to those skilled in the art.

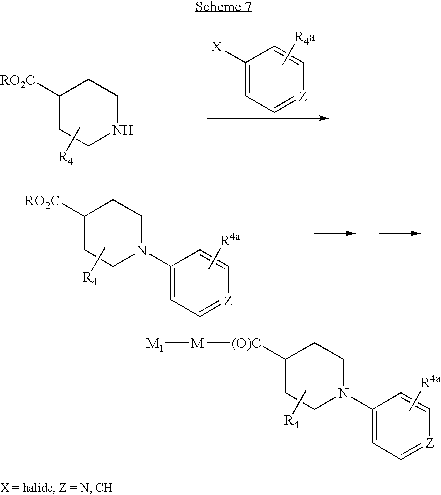

Compounds of the present invention may be synthesized following the general outline of Scheme 8.

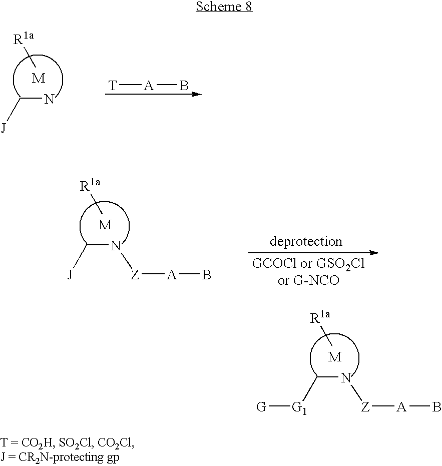

Compounds 9 and 10 can be obtained by the reaction sequence outlined below. The starting intermediate, methyl 4-(2-oxopyridin-1(2H)-yl)benzoate 3, can be obtained by coupling reaction between methyl 4-iodo benzoate and 2-hydroxy pyridine (see Scheme 9). The corresponding acid chloride 4, which can be obtained by classical methods from the parent ester 3, can then be reacted with an appropriate amino compound 5 and/or 6 in CH₂Cl₂ and in the presence of triethylamine (TEA) to yield compounds 7 and/or 8. Compounds 7 and/or 8 can then be deprotected under acidic conditions and reacted with a desired aroyl chloride to yield compounds of the present invention (9 and 10).

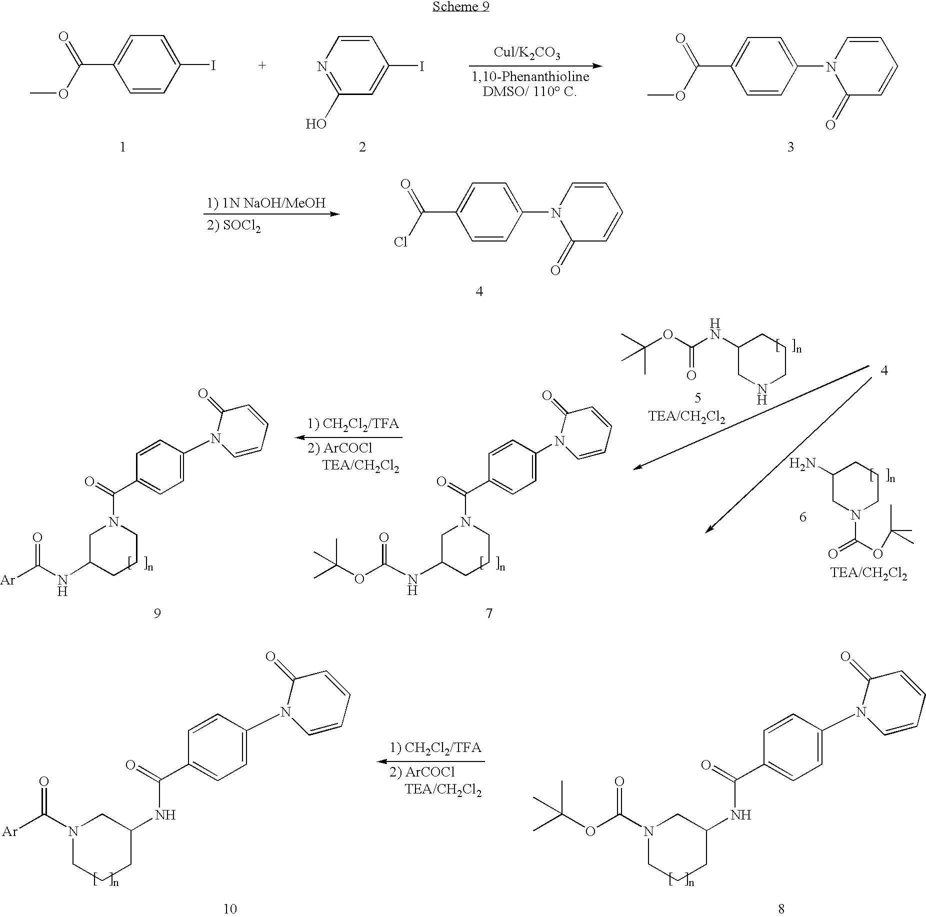

As shown in Scheme 10, amino compound 6 can be reacted with the appropriated isocyanate 11 to yield derivative 12. Derivative 12 can then be modified via deprotection and reaction with an active acid as shown above to provide compounds of the present invention.

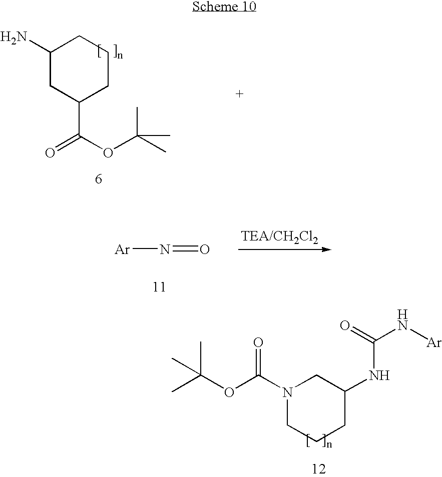

One stereoisomer of a compound of the present invention may display superior activity compared with the other. Compounds of the present invention may be chiral and accordingly in various enantiomeric forms. They therefore may exist in racemic or in optically active form. Thus, compounds of formulae A-D are also part of the present invention.

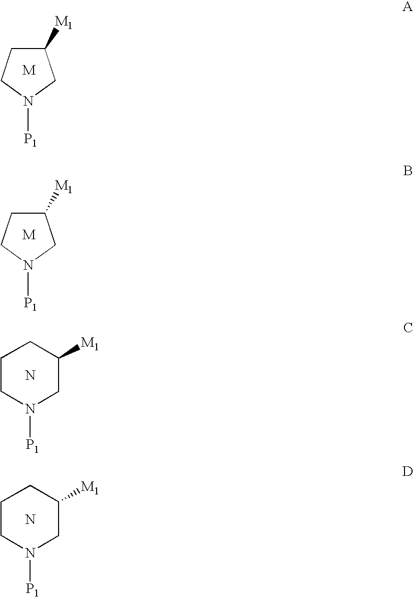

When required, separation of the racemic material can be achieved by HPLC using a chiral column or by a resolution using a resolving agent such as described in Wilen, S. H. Tables of Resolving Agents and Optical Resolutions 1972, 308 or using enantiomerically pure acids and bases. A chiral compound of the present invention may also be directly synthesized using a chiral catalyst or a chiral ligand, e.g., Jacobsen, E. Acc. Chem. Res. 2000, 33, 421-431 or using other enantio- and diastereo-selective reactions and reagents known to one skilled in the art of asymmetric synthesis.

Utility

The compounds of this invention are inhibitors of factor Xa and are useful as anticoagulants for the treatment or prevention of thromboembolic disorders in mammals (i.e., factor Xa-associated disorders). In general, a thromboembolic disorder is a circulatory disease caused by blood clots (i.e., diseases involving fibrin formation, platelet activation, and/or platelet aggregation). The term “thromboembolic disorders” as used herein includes arterial cardiovascular thromboembolic disorders, venous cardiovascular thromboembolic disorders, and thromboembolic disorders in the chambers of the heart. The term “thromboembolic disorders” as used herein also includes specific disorders selected from, but not limited to, unstable angina or other acute coronary syndromes, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis. It is noted that thrombosis includes occlusion (e.g. after a bypass) and reocclusion (e.g., during or after percutaneous transluminal coronary angioplasty). The thromboembolic disorders may result from conditions including but not limited to atherosclerosis, surgery or surgical complications, prolonged immobilization, arterial fibrillation, congenital thrombophilia, cancer, diabetes, effects of medications or hormones, and complications of pregnancy. The anticoagulant effect of compounds of the present invention is believed to be due to inhibition of factor Xa or thrombin.

The effectiveness of compounds of the present invention as inhibitors of factor Xa was determined using purified human factor Xa and synthetic substrate. The rate of factor Xa hydrolysis of chromogenic substrate S2222 (Diapharma/Chromogenix, West Chester, Ohio) was measured both in the absence and presence of compounds of the present invention. Hydrolysis of the substrate resulted in the release of pNA, which was monitored spectrophotometrically by measuring the increase in absorbance at 405 nm. A decrease in the rate of absorbance change at 405 nm in the presence of inhibitor is indicative of enzyme inhibition. The results of this assay are expressed as inhibitory constant, Kᵢ.

Factor Xa determinations were made in 0.10 M sodium phosphate buffer, pH 7.5, containing 0.20 M NaCl, and 0.5% PEG 8000. The Michaelis constant, Kₘ, for substrate hydrolysis was determined at 25° C. using the method of Lineweaver and Burk. Values of Kᵢ were determined by allowing 0.2-0.5 nM human factor Xa (Enzyme Research Laboratories, South Bend, Ind.) to react with the substrate (0.20 mM-1 mM) in the presence of inhibitor. Reactions were allowed to go for 30 min and the velocities (rate of absorbance change vs. time) were measured in the time frame of 25-30 min. The following relationship was used to calculate Kᵢ values:
(vₒ−vₛ)/vₛ=I/(Kᵢ(1+S/Kₘ))
where:
vₒ is the velocity of the control in the absence of inhibitor;
vₛ is the velocity in the presence of inhibitor;
I is the concentration of inhibitor;
Kᵢ is the dissociation constant of the enzyme:inhibitor complex;
S is the concentration of substrate;
Kₘ is the Michaelis constant.

Compounds tested in the above assay are considered to be active if they exhibit a Kᵢ of ≦10 μM. Preferred compounds of the present invention have Kᵢ's of ≦1 μM. More preferred compounds of the present invention have Kᵢ's of ≦0.1 μM. Even more preferred compounds of the present invention have Kᵢ's of ≦0.01 μM. Still more preferred compounds of the present invention have Kᵢ's of ≦0.001 μM. Using the methodology described above, a number of compounds of the present invention were found to exhibit Kᵢ's of ≦10 μM, thereby confirming the utility of the compounds of the present invention as effective Xa inhibitors.

The antithrombotic effect of compounds of the present invention can be demonstrated in a rabbit arterio-venous (AV) shunt thrombosis model. In this model, rabbits weighing 2-3 kg anesthetized with a mixture of xylazine (10 mg/kg i.m.) and ketamine (50 mg/kg i.m.) are used. A saline-filled AV shunt device is connected between the femoral arterial and the femoral venous cannulae. The AV shunt device consists of a piece of 6-cm tygon tubing that contains a piece of silk thread. Blood will flow from the femoral artery via the AV-shunt into the femoral vein. The exposure of flowing blood to a silk thread will induce the formation of a significant thrombus. After 40 min, the shunt is disconnected and the silk thread covered with thrombus is weighed. Test agents or vehicle will be given (i.v., i.p., s.c., or orally) prior to the opening of the AV shunt. The percentage inhibition of thrombus formation is determined for each treatment group. The ID₅₀ values (dose which produces 50% inhibition of thrombus formation) are estimated by linear regression.

The compounds of the present invention may also be useful as inhibitors of serine proteases, notably human thrombin, Factor VIIa, Factor IXa, Factor XIa, urokinase, plasma kallikrein, and plasmin. Because of their inhibitory action, these compounds are indicated for use in the prevention or treatment of physiological reactions, blood coagulation and inflammation, catalyzed by the aforesaid class of enzymes. Specifically, the compounds have utility as drugs for the treatment of diseases arising from elevated thrombin activity such as myocardial infarction, and as reagents used as anticoagulants in the processing of blood to plasma for diagnostic and other commercial purposes.

Some compounds of the present invention were shown to be direct acting inhibitors of the serine protease thrombin by their ability to inhibit the cleavage of small molecule substrates by thrombin in a purified system. In vitro inhibition constants were determined by the method described by Kettner et al. in J. Biol. Chem. 265, 18289-18297 (1990), herein incorporated by reference. In these assays, thrombin-mediated hydrolysis of the chromogenic substrate S2238 (Helena Laboratories, Beaumont, Tex.) was monitored spectrophotometrically. Addition of an inhibitor to the assay mixture results in decreased absorbance and is indicative of thrombin inhibition. Human thrombin (Enzyme Research Laboratories, Inc., South Bend, Ind.) at a concentration of 0.2 nM in 0.10 M sodium phosphate buffer, pH 7.5, 0.20 M NaCl, and 0.5% PEG 6000, was incubated with various substrate concentrations ranging from 0.20 to 0.02 mM. After 25 to 30 min of incubation, thrombin activity was assayed by monitoring the rate of increase in absorbance at 405 nm that arises owing to substrate hydrolysis. Inhibition constants were derived from reciprocal plots of the reaction velocity as a function of substrate concentration using the standard method of Lineweaver and Burk. Using the methodology described above, some compounds of this invention were evaluated and found to exhibit a Kᵢ of less than 10 μm, thereby confirming the utility of the compounds of the present invention as effective thrombin inhibitors.

The compounds are administered to a mammal in a therapeutically effective amount. By “therapeutically effective amount” it is meant an amount of a compound of the present invention that, when administered alone or in combination with an additional therapeutic agent to a mammal, is effective to treat a thromboembolic condition or disease.

The compounds of the present invention can be administered alone or in combination with one or more additional therapeutic agents. By “administered in combination” or “combination therapy” it is meant that a compound of the present invention and one or more additional therapeutic agents are administered concurrently to the mammal being treated. When administered in combination each component may be administered at the same time or sequentially in any order at different points in time. Thus, each component may be administered separately but sufficiently closely in time so as to provide the desired therapeutic effect.

Additional therapeutic agents include other anti-coagulant or coagulation inhibitory agents, anti-platelet or platelet inhibitory agents, thrombin inhibitors, thrombolytic or fibrinolytic agents, anti-arrythmic agents, anti-hypertensive agents, calcium channel blockers (L-type and T-type), cardiac glycosides, diruetics, mineralocorticoid receptor antagonists, phospodiesterase inhibitors, cholesterol/lipid lowering agents and lipid profile therapies, anti-diabetic agents, anti-depressants, anti-inflammatory agents (steroidal and non-steroidal), anti-osteoporosis agents, hormone replacement therapies, oral contraceptives, anti-obesity agents, anti-anxiety agents, anti-proliferative agents, anti-tumor agents, anti-ulcer and gastroesophageal reflux disease agents, growth hormone and/or growth hormone secretagogues, thyroid mimetics (including thyroid receptor antagonist), anti-infective agents, anti-viral agents, anti-bacterial agents, and anti-fungal agents.

Other anticoagulant agents (or coagulation inhibitory agents) that may be used in combination with the compounds of this invention include warfarin and heparin (either unfractionated heparin or any commercially available low molecular weight heparin), synthetic pentasaccharide, direct acting thrombin inhibitors including hirudin and argatrobanas well as other factor Xa inhibitors such as those described in the publications identified above under Background of the Invention.

The term anti-platelet agents (or platelet inhibitory agents), as used herein, denotes agents that inhibit platelet function, for example by inhibiting the aggregation, adhesion or granular secretion of platelets. Agents include, but are not limited to, the various known non-steroidal anti-inflammatory drugs (NSAIDS) such as aspirin, ibuprofen, naproxen, sulindac, indomethacin, mefenamate, droxicam, diclofenac, sulfinpyrazone, piroxicam, and pharmaceutically acceptable salts or prodrugs thereof. Of the NSAIDS, aspirin (acetylsalicyclic acid or ASA) and piroxicam are preferred. Other suitable platelet inhibitory agents include IIb/IIIa antagonists (e.g., tirofiban, eptifibatide, and abciximab), thromboxane-A2-receptor antagonists (e.g., ifetroban), thromboxane-A2-synthetase inhibitors, PDE-III inhibitors (e.g., dipyridamole), and pharmaceutically acceptable salts or prodrugs thereof.

The term anti-platelet agents (or platelet inhibitory agents), as used herein, is also intended to include ADP (adenosine diphosphate) receptor antagonists, preferably antagonists of the purinergic receptors P₂Y₁ and P₂Y₁₂, with P₂Y₁₂ being even more preferred. Preferred P₂Y₁₂ receptor antagonists include ticlopidine and clopidogrel, including pharmaceutically acceptable salts or prodrugs thereof. Clopidogrel is an even more preferred agent. Ticlopidine and clopidogrel are also preferred compounds since they are known to be gentle on the gastro-intestinal tract in use.

The term thrombin inhibitors (or anti-thrombin agents), as used herein, denotes inhibitors of the serine protease thrombin. By inhibiting thrombin, various thrombin-mediated processes, such as thrombin-mediated platelet activation (that is, for example, the aggregation of platelets, and/or the granular secretion of plasminogen activator inhibitor-1 and/or serotonin) and/or fibrin formation are disrupted. A number of thrombin inhibitors are known to one of skill in the art and these inhibitors are contemplated to be used in combination with the present compounds. Such inhibitors include, but are not limited to, boroarginine derivatives, boropeptides, heparins, hirudin, argatroban, and melagatran, including pharmaceutically acceptable salts and prodrugs thereof. Boroarginine derivatives and boropeptides include N-acetyl and peptide derivatives of boronic acid, such as C-terminal α-aminoboronic acid derivatives of lysine, ornithine, arginine, homoarginine and corresponding isothiouronium analogs thereof. The term hirudin, as used herein, includes suitable derivatives or analogs of hirudin, referred to herein as hirulogs, such as disulfatohirudin.

The term thrombolytics or fibrinolytic agents (or thrombolytics or fibrinolytics), as used herein, denote agents that lyse blood clots (thrombi). Such agents include tissue plasminogen activator (natural or recombinant) and modified forms thereof, anistreplase, urokinase, streptokinase, tenecteplase (TNK), lanoteplase (nPA), factor VIIa inhibitors, PAI-1 inhibitors (i.e., inactivators of tissue plasminogen activator inhibitors), alpha2-antiplasmin inhibitors, and anisoylated plasminogen streptokinase activator complex, including pharmaceutically acceptable salts or prodrugs thereof. The term anistreplase, as used herein, refers to anisoylated plasminogen streptokinase activator complex, as described, for example, in EP 028,489, the disclosure of which is hereby incorporated herein by reference herein. The term urokinase, as used herein, is intended to denote both dual and single chain urokinase, the latter also being referred to herein as prourokinase.

Examples of suitable anti-arrythmic agents for use in combination with the present compounds include: Class I agents (such as propafenone); Class II agents (such as carvadiol and propranolol); Class III agents (such as sotalol, dofetilide, amiodarone, azimilide and ibutilide); Class IV agents (such as ditiazem and verapamil); K⁺ channel openers such as I_{Ach} inhibitors, and I_{Kur} inhibitors (e.g., compounds such as those disclosed in WO01/40231).

Examples of suitable anti-hypertensive agents for use in combination with the compounds of the present invention include: alpha adrenergic blockers; beta adrenergic blockers; calcium channel blockers (e.g., diltiazem, verapamil, nifedipine, amlodipine and mybefradil); diruetics (e.g., chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetamide, triamtrenene, amiloride, spironolactone); renin inhibitors; ACE inhibitors (e.g., captopril, zofenopril, fosinopril, enalapril, ceranopril, cilazopril, delapril, pentopril, quinapril, ramipril, lisinopril); AT-1 receptor antagonists (e.g., losartan, irbesartan, valsartan); ET receptor antagonists (e.g., sitaxsentan, atrsentan and compounds disclosed in U.S. Pat. Nos. 5,612,359 and 6,043,265); Dual ET/AII antagonist (e.g., compounds disclosed in WO 00/01389); neutral endopeptidase (NEP) inhibitors; vasopepsidase inhibitors (dual NEP-ACE inhibitors) (e.g., omapatrilat, gemopatrilat and nitrates).

Examples of suitable calcium channel blockers (L-type or T-type) for use in combination with the compounds of the present invention include diltiazem, verapamil, nifedipine, amlodipine and mybefradil.

Examples of suitable cardiac glycosides for use in combination with the compounds of the present invention include digitalis and ouabain.

Examples of suitable diruetics for use in combination with the compounds of the present invention include: chlorothiazide, hydrochlorothiazide, flumethiazide, hydroflumethiazide, bendroflumethiazide, methylchlorothiazide, trichloromethiazide, polythiazide, benzthiazide, ethacrynic acid tricrynafen, chlorthalidone, furosemide, musolimine, bumetamide, triamtrenene, amiloride, and spironolactone.

Examples of suitable mineralocorticoid receptor antagonists for use in combination with the compounds of the present invention include sprionolactone and eplirinone.

Examples of suitable phospodiesterase inhibitors for use in combination with the compounds of the present invention include: PDE III inhibitors (such as cilostazol); and PDE V inhibitors (such as sildenafil).

Examples of suitable cholesterol/lipid lowering agents and lipid profile therapies for use in combination with the compounds of the present invention include: HMG-CoA reductase inhibitors (e.g., pravastatin, lovastatin, atorvastatin, simvastatin, fluvastatin, NK-104 (a.k.a. itavastatin, or nisvastatin or nisbastatin) and ZD-4522 (a.k.a. rosuvastatin, or atavastatin or visastatin)); squalene synthetase inhibitors; fibrates; bile acid sequestrants (such as questran); ACAT inhibitors; MTP inhibitors; lipooxygenase inhibitors; choesterol absorption inhibitors; and cholesterol ester transfer protein inhibitors (e.g., CP-529414).

Examples of suitable anti-diabetic agents for use in combination with the compounds of the present invention include: biguanides (e.g., metformin); glucosidase inhibitors (e.g., acarbose); insulins (including insulin secretagogues or insulin sensitizers); meglitinides (e.g., repaglinide); sulfonylureas (e.g., glimepiride, glyburide and glipizide); biguanide/glyburide combinations (e.g., glucovance), thiozolidinediones (e.g., troglitazone, rosiglitazone and pioglitazone), PPAR-alpha agonists, PPAR-gamma agonists, PPAR alpha/gamma dual agonists, SGLT2 inhibitors, inhibitors of fatty acid binding protein (aP2) such as those disclosed in WO00/59506, glucagon-like peptide-1 (GLP-1), and dipeptidyl peptidase IV (DP4) inhibitors.

Examples of suitable anti-depressant agents for use in combination with the compounds of the present invention include nefazodone and sertraline.

Examples of suitable anti-inflammatory agents for use in combination with the compounds of the present invention include: prednisone; dexamethasone; enbrel; protien tyrosine kinase (PTK) inhibitors; cyclooxygenase inhibitors (including NSAIDs, and COX-1 and/or COX-2 inhibitors); aspirin; indomethacin; ibuprofen; prioxicam; naproxen; celecoxib; and/or rofecoxib.

Examples of suitable anti-osteoporosis agents for use in combination with the compounds of the present invention include alendronate and raloxifene.

Examples of suitable hormone replacement therapies for use in combination with the compounds of the present invention include estrogen (e.g., congugated estrogens) and estradiol.

Examples of suitable anti-coagulants for use in combination with the compounds of the present invention include heparins (e.g., unfractioned and low molecular weight heparins such as enoxaparin and dalteparin).

Examples of suitable anti-obesity agents for use in combination with the compounds of the present invention include orlistat and aP2 inhibitors (such as those disclosed in WO00/59506).

Examples of suitable anti-anxiety agents for use in combination with the compounds of the present invention include diazepam, lorazepam, buspirone, and hydroxyzine pamoate.

Examples of suitable anti-proliferative agents for use in combination with the compounds of the present invention include cyclosporin A, paclitaxel, adriamycin; epithilones, cisplatin, and carboplatin.

Examples of suitable anti-ulcer and gastroesophageal reflux disease agents for use in combination with the compounds of the present invention include famotidine, ranitidine, and omeprazole.

Administration of the compounds of the present invention (i.e., a first therapeutic agent) in combination with at least one additional therapeutic agent (i.e., a second therapeutic agent), preferably affords an efficacy advantage over the compounds and agents alone, preferably while permitting the use of lower doses of each (i.e., a synergistic combination). A lower dosage minimizes the potential of side effects, thereby providing an increased margin of safety. It is preferred that at least one of the therapeutic agents is administered in a sub-therapeutic dose. It is even more preferred that all of the therapeutic agents be administered in sub-therapeutic doses. Sub-therapeutic is intended to mean an amount of a therapeutic agent that by itself does not give the desired therapeutic effect for the condition or disease being treated. Synergistic combination is intended to mean that the observed effect of the combination is greater than the sum of the individual agents administered alone.

The compounds of the present invention are also useful as standard or reference compounds, for example as a quality standard or control, in tests or assays involving the inhibition of factor Xa. Such compounds may be provided in a commercial kit, for example, for use in pharmaceutical research involving factor Xa. For example, a compound of the present invention could be used as a reference in an assay to compare its known activity to a compound with an unknown activity. This would ensure the experimenter that the assay was being performed properly and provide a basis for comparison, especially if the test compound was a derivative of the reference compound. When developing new assays or protocols, compounds according to the present invention could be used to test their effectiveness.

The compounds of the present invention may also be used in diagnostic assays involving factor Xa. For example, the presence of factor Xa in an unknown sample could be determined by addition of chromogenic substrate S2222 to a series of solutions containing test sample and optionally one of the compounds of the present invention. If production of pNA is observed in the solutions containing test sample, but not in the presence of a compound of the present invention, then one would conclude factor Xa was present.

Compounds of the present invention may further be useful as diagnostic agents and adjuncts. For example, the present compounds may be useful in maintaining whole and fractionated blood in the fluid phase such as required for analytical and biological testing.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises: a first therapeutic agent, comprising: a compound of the present invention or a pharmaceutically acceptable salt form thereof, and (c) a package insert stating that the pharmaceutical composition can be used for the treatment of a thromboembolic disorder (as defined previously). In another embodiment, the package insert states that the pharmaceutical composition can be used in combination (as defined previously) with a second therapeutic agent to treat a thromboembolic disorder. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

Dosage and Formulation

The compounds of this invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress of the thromboembolic disorder.

By way of general guidance, the daily oral dosage of each active ingredient, when used for the indicated effects, will range between about 0.001 to 1000 mg/kg of body weight, preferably between about 0.01 to 100 mg/kg of body weight per day, and most preferably between about 1.0 to 20 mg/kg/day. Intravenously, the most preferred doses will range from about 1 to about 10 mg/kg/min during a constant rate infusion. Compounds of this invention may be administered in a single daily dose, or the total daily dosage may be administered in divided doses of two, three, or four times daily.

Compounds of this invention can be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as pharmaceutical carriers) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 1 milligram to about 100 milligrams of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance.

In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl-or propyl-paraben, and chlorobutanol.

Suitable pharmaceutical carriers are described in Remington's Pharmaceutical Sciences, 18th ed., Mack Publishing Company, Easton, Pa., 1990, a standard reference text in this field.

Where the compounds of this invention are combined with other anticoagulant agents, for example, a daily dosage may be about 0.1 to 100 milligrams of the compound of the present invention and about 1 to 7.5 milligrams of the second anticoagulant, per kilogram of patient body weight. For a tablet dosage form, the compounds of this invention generally may be present in an amount of about 5 to 10 milligrams per dosage unit, and the second anti-coagulant in an amount of about 1 to 5 milligrams per dosage unit.

Where the compounds of the present invention are administered in combination with an anti-platelet agent, by way of general guidance, typically a daily dosage may be about 0.01 to 25 milligrams of the compound of the present invention and about 50 to 150 milligrams of the anti-platelet agent, preferably about 0.1 to 1 milligrams of the compound of the present invention and about 1 to 3 milligrams of antiplatelet agents, per kilogram of patient body weight.

Where the compounds of Formula I are administered in combination with thrombolytic agent, typically a daily dosage may be about 0.1 to 1 milligrams of the compound of Formula I, per kilogram of patient body weight and, in the case of the thrombolytic agents, the usual dosage of the thrombolyic agent when administered alone may be reduced by about 70-80% when administered with a compound of Formula I.

Where two or more of the foregoing second therapeutic agents are administered with the compound of Formula I, generally the amount of each component in a typical daily dosage and typical dosage form may be reduced relative to the usual dosage of the agent when administered alone, in view of the additive or synergistic effect of the therapeutic agents when administered in combination.

Particularly when provided as a single dosage unit, the potential exists for a chemical interaction between the combined active ingredients. For this reason, when the compound of the present invention and a second therapeutic agent are combined in a single dosage unit they are formulated such that although the active ingredients are combined in a single dosage unit, the physical contact between the active ingredients is minimized (that is, reduced). For example, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a material that affects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low viscosity grade of hydroxypropyl methylcellulose (HPMC) or other appropriate materials as known in the art, in order to further separate the active components. The polymer coating serves to form an additional barrier to interaction with the other component.

These as well as other ways of minimizing contact between the components of combination products of the present invention, whether administered in a single dosage form or administered in separate forms but at the same time by the same manner, will be readily apparent to those skilled in the art, once armed with the present disclosure.

Other features of the invention will become apparent in the course of the following descriptions of exemplary embodiments that are afforded for illustration of the invention and are not intended to be limiting thereof.

### EXAMPLES

### Example 1

### 6-Chloro-naphthalene-2-sulfonic Acid [1-(3,4,5,6-tetrahydro-2H-[1,4′]bipyridinyl-4-carbonyl)-pyrrolidin-3-ylmethyl]-amide

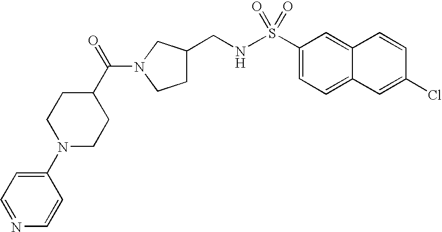

Part A: To a solution of the (1-benzyl-pyrrolidin-3-yl)-methylamine (256 mg, 1.34 mmol) in dichloromethane (10 mL) at 0° C. under nitrogen were slowly added trifloroacetic anhydride (0.194 mL, 1.34 mmol) and triethylamine (0.225 mL, 1.34 mmol). The reaction mixture was stirred at RT for 30 min. The LC-MS showed the disappearance of starting material and formation of product. The reaction was quenched by H₂O, and extracted with dichloromethane (3×10 mL). The combined organic layers were dried over sodium sulfate and filtered. The solvent was removed providing N-(1-benzyl-pyrrolidin-3-ylmethyl)-2,2,2-trifluoro-acetamide (380 mg, 98.7% yield), which was used in the next Part without further purification.

Part B: To a solution of the product obtained above (380 mg, 1.33 mmol) in methanol (20 mL) was added palladium on carbon (10%). The reaction mixture was stirred under 1 atmosphere of hydrogen for 2 hours, then the flask was purged with nitrogen and the reaction mixture was filtered through a pad of Celite®. The solvent was removed to give 2,2,2-trifluoro-N-pyrrolidin-3-ylmethyl-acetamide as a colorless oil (260 mg, 100% yield).

Part C: To a solution of the product obtained above (620 mg, 3.00 mmol) in dichloromethane/DMF (10 mL/2 mL) were sequentially added 3,4,5,6-Tetrahydro-2H-[1,4′]bipyridinyl-4-carboxylic acid (590 mg, 3.00 mmol), 1-(3-dimethylaminopropyl)-2-ethylcarbodiimide hydrochloride (WSC) (690 mg, 3.59 mmol), triethylamine (0.502 mL, 3.60 mmol), and a catalytic amount of 4-dimethyl-aminopyridine. The reaction mixture was stirred for 24 h at 42° C., quenched by addition of water, and extracted with dichloromethane (3×20 mL). The organic fraction was concentrated, and the residue was purified by flash chromatography on silica gel, eluting with menthol/acetate (30/70) to give 2,2,2-trifluoro-N-[1-(3,4,5,6-tetrahydro-2H-[1,4′]bipyridinyl-4-carbonyl)-pyrrolidin-3-ylmethyl]acetamide as a white solid (520 mg, 48%).

Part D: To the product obtained above (100 mg, 0.26 mmol) was added 7N NH₃ (10 mL) and the reaction mixture was stirred at RT for 18 hr. The NH₃ was removed by vacuum. The solvent was then removed to provide (3-aminomethyl-pyrrolidin-1-yl)-(3,4,5,6-tetrahydro-2H-[1,4′]bipyridinyl-4-yl)-methanone (40 mg, 53% yield) as a white oil.

Part E: To a solution of the product obtained above (15 mg, 0.052 mmol) in dichloromethane (1 mL) at rt was added 6-chloronaphthalene-2-sulfonyl chloride (13.5 mg, 0.52 mmol) and triethylamine (7.2 mL, 0.052 mmol). The reaction mixture was stirred at rt for 30 hr. The solvent was removed, and the crude material was purified by the preparative HPLC to provide the title compound (2.8 mg, 10% yield) as the white solid. MS found: (M+1)⁺=514.

### Example 2

### 4-Methyl-2-pyridin-4-yl-thiazole-5-carboxylic Acid [1-(6-chloro-naphthalene-2-sulfonyl)-pyrrolidin-3-ylmethyl]-amide

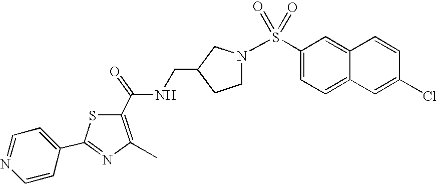

Part A: Following a procedure analogous to that described in Example 1, Part A, N-[1-(6-chloro-naphthalene-2-sulfonyl)-pyrrolidin-3-ylmethyl]-2,2,2-trifluoro-acetamide was obtained.

Part B: To the product obtained above (58.3 mg, 0.139 mmol) in methanol was added aqueous K₂CO₃ (287 mg, 2.08 mmol). The reaction mixture was stirred at RT for 18 hr. The methanol was removed by vacuum. The solution was extracted with EtOAc (3×20 mL). The combined organic layers were dried over sodium sulfate and filtered. The solvent was removed providing 1-(6-Chloro-naphthalene-2-sulfonyl)-pyrrolidin-3-yl]-methylamine (44 mg, 97% yield) that was used for the next Part without further purification.

Part C: Following a procedure analogous to that described in Example 1, Part C, the title compound was obtained. MS found: (M+1)⁺=527.

### Example 3

### 5-Chloro-3-methyl-benzo[b]thiophene-2-sulfonic Acid [1-(2′-methanesulfonyl-biphenyl-4-carbonyl)-pyrrolidin-3-ylmethyl]-amide

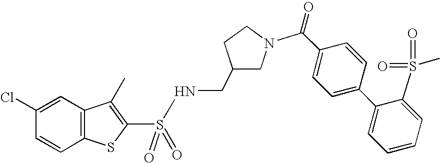

Following a procedure analogous to that described in Example 1, the title compound was obtained as a white solid. MS found: (M+1)⁺=603.

### Example 4

### 4′-Chloro-biphenyl-3-sulfonic Acid [1-(2′-methanesulfonyl-biphenyl-4-carbonyl)-pyrrolidin-3-yl]-amide

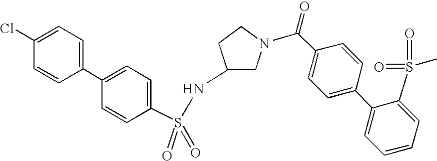

Following a procedure analogous to that described in Example 1, the title compound was obtained as a white solid. MS found: (M+1)⁺=595.

### Example 5

### 6-Chloro-naphthalene-2-sulfonic Acid [1-(4-methyl-2-pyridin-4-yl-thiazole-5-carbonyl)-pyrrolidin-3-ylmethyl]-amide

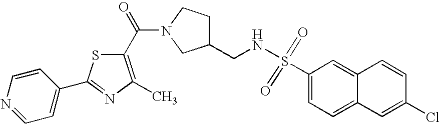

Following a procedure analogous to that described in Example 1, the title compound was obtained as a yellow solid. MS found: (M+1)⁺=527.

### Example 6

### 3′,4′-Dichloro-biphenyl-4-sulfonic Acid [1-(2′-methanesulfonyl-biphenyl-4-carbonyl)-pyrrolidin-3-ylmethyl]-amide

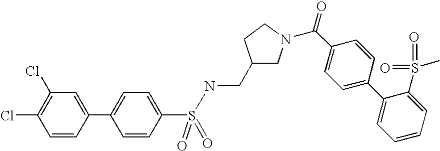

Following a procedure analogous to that described in Example 1, the title compound was obtained as a white solid. MS found: (M+1)⁺=643.

### Example 7

### 2-(4-Chloro-phenyl)-ethenesulfonic Acid [1-(3,4,5,6-tetrahydro-2H-[1,4′]bipyridinyl-4-carbonyl)-pyrrolidin-3-ylmethyl]-amide

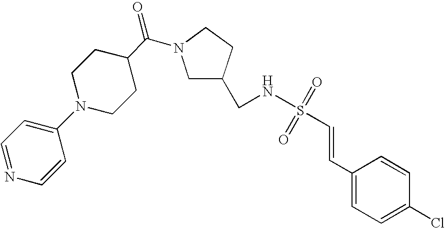

Following a procedure analogous to that described in Example 1, the title compound was obtained. MS found: (M+1)⁺=489.

### Example 8

### 5-Pyridin-2-yl-thiophene-2-sulfonic Acid [1-(2′-methanesulfonyl-biphenyl-4-carbonyl)-pyrrolidin-3-ylmethyl]-amide

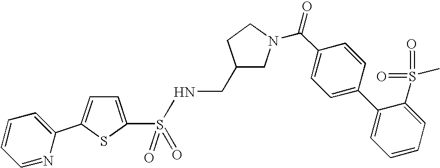

Following a procedure analogous to that described in Example 1, the title compound was obtained as a white solid. MS found: (M+1)⁺=581.

### Example 9

### 2-(3-Chloro-phenyl)-ethenesulfonic Acid [1-(3,4,5,6-tetrahydro-2H-[1,4′]bipyridinyl-4-carbonyl)-pyrrolidin-3-ylmethyl]-amide

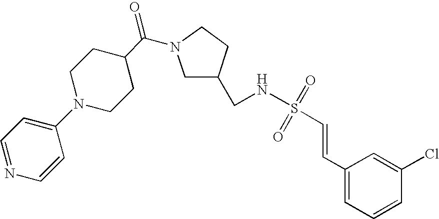

Following a procedure analogous to that described in Example 1, the title compound was obtained. MS found: (M+1)⁺=489.

### Example 10

### 2-(5-Bromo-thiophen-2-yl)-ethenesulfonic Acid [1-(3,4,5,6-tetrahydro-2H-[1,4′]bipyridinyl-4-carbonyl)-pyrrolidin-3-ylmethyl]-amide

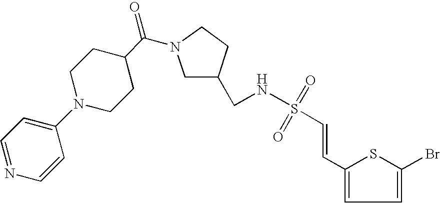

Following a procedure analogous to that described in Example 1, the title compound was obtained. MS found: (M+1)⁺=539.

### Example 11

### 2-Phenyl-ethenesulfonic Acid [1-(3,4,5,6-tetrahydro-2H-[1,4′]bipyridinyl-4-carbonyl)-pyrrolidin-3-ylmethyl]-amide

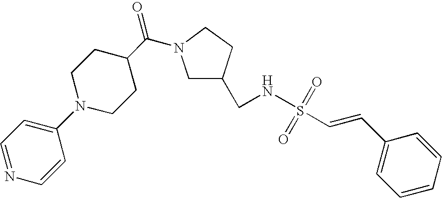

Following a procedure analogous to that described in Example 1, the title compound was obtained. MS found: (M+1)⁺=455.

### Example 12

### 6-Chloro-benzo[b]thiophene-2-sulfonic Acid [1-(3,4,5,6-tetrahydro-2H-[1,4′]bipyridinyl-4-carbonyl)-pyrrolidin-3-ylmethyl]-amide

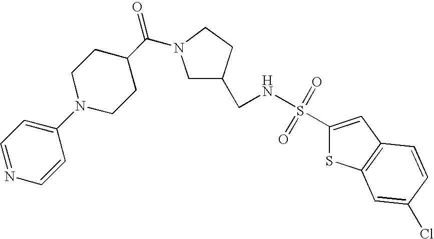

Following a procedure analogous to that described in Example 1, the title compound was obtained. MS found: (M+1)⁺=519.

### Example 13

### 2-(5-Bromo-thiophen-2-yl)-ethenesulfonic Acid [1-(3,4,5,6-tetrahydro-2H-[1,4′]bipyridinyl-4-carbonyl)-pyrrolidin-3-(R)-ylmethyl]-amide

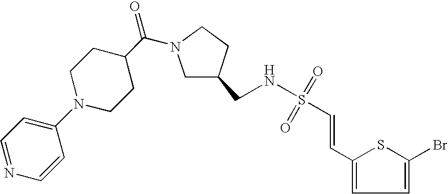

Following a procedure analogous to that described in Example 1, the title compound was obtained. MS found: (M+1)⁺=539.

### Example 14

### (S)-6-Bromo-naphthalene-2-sulfonic Acid [1-(1-benzoyl-piperidine-4-carbonyl)-pyrrolidin-3-ylmethyl]-amide

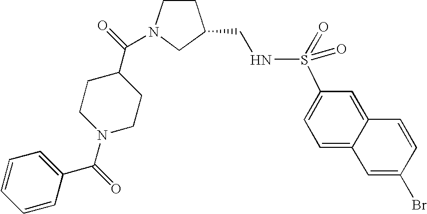

Following a procedure analogous to that described in Example 1, the title compound was obtained. MS found: (M+1)⁺=584.

### Example 15

### 5′-Chloro-[2,2′]bithiophenyl-5-sulfonic Acid [1-(3,4,5,6-tetrahydro-2H-[1,4′]bipyridinyl-4-carbonyl)-pyrrolidin-3-yl]-amide

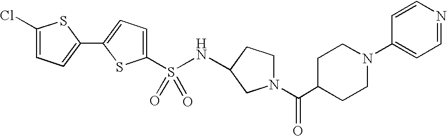

Following a procedure analogous to that described in Example 1, the title compound was obtained. MS found: (M+1)⁺=537.

### Example 16

### 5-Chloro-benzo[b]thiophene-2-sulfonic Acid [1-(3,4,5,6-tetrahydro-2H-[1,4′]bipyridinyl-4-carbonyl)-pyrrolidin-3-yl]-amide

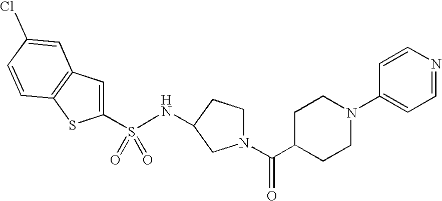

Following a procedure analogous to that described in Example 1, the title compound was obtained. MS found: (M+1)⁺=505.

### Example 17

### 5-Chloro-benzo[b]thiophene-2-sulfonic Acid [1-(3,4,5,6-tetrahydro-2H-[1,4′]bipyridinyl-4-carbonyl)-pyrrolidin-2-ylmethyl]-amide

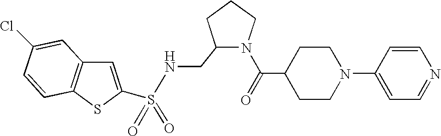

Following a procedure analogous to that described in Example 1, the title compound was obtained. MS found: (M+1)⁺=519.

### Example 18

### 5′-Chloro-[2,2′]bithiophenyl-5-sulfonic Acid [1-(3,4,5,6-tetrahydro-2H-[1,4′]bipyridinyl-4-carbonyl)-pyrrolidin-2-ylmethyl]-amide

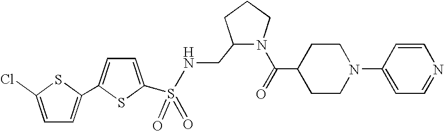

Following a procedure analogous to that described in Example 1, the title compound was obtained. MS found: (M+1)⁺=551.

### Example 19

### 3,4,5,6-Tetrahydro-2H-[1,4′]bipyridinyl-4-carboxylic Acid [1-(5′-chloro-[2,2′]bithiophenyl-5-sulfonyl)-pyrrolidin-2-ylmethyl]-amide

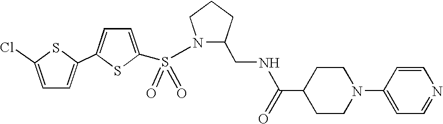

Following a procedure analogous to that described in Example 1, the title compound was obtained. MS found: (M+1)⁺=551.

### Example 20

### (R)-5-Chloro-benzo[b]thiophene-2-sulfonic Acid [1-(3,4,5,6-tetrahydro-2H-[1,4′]bipyridinyl-4-carbonyl)-pyrrolidin-3-ylmethyl]-amide

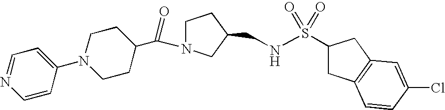

Following a procedure analogous to that described in Example 1, the title compound was obtained. MS found: (M+1)⁺=519.

### Example 21

### (R)-2-(5-Chloro-thiophen-2-yl)-ethenesulfonic Acid [1-(3,4,5,6-tetrahydro-2H-[1,4′]bipyridinyl-4-carbonyl)-pyrrolidin-3-ylmethyl]-amide

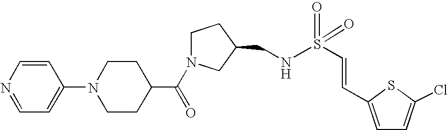

Following a procedure analogous to that described in Example 1, the title compound was obtained. MS found: (M+1)⁺=495.

### Example 22

### (R)-4′-(3-{[2-(5-Chloro-thiophen-2-yl)-ethenesulfonylamino]-methyl}-pyrrolidine-1-carbonyl)-biphenyl-2-sulfonic Acid Amide

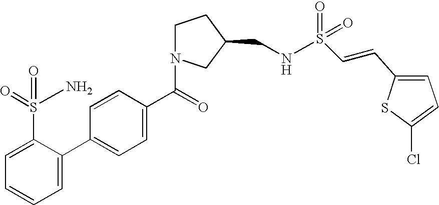

Following a procedure analogous to that described in Example 1, the title compound was obtained. MS found: (M+1)⁺=566.

### Example 23

### (S)-2-(5-Chloro-thiophen-2-yl)-ethenesulfonic Acid {1-[1-(4-trifluoromethyl-pyrimidin-2-yl)-piperidine-4-carbonyl]-pyrrolidin-3-ylmethyl}-amide

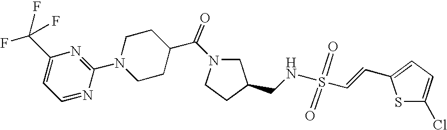

Following a procedure analogous to that described in Example 1, the title compound was obtained. MS found: (M+1)⁺=564.

### Example 24

### (S)-2-(5-Chloro-thiophen-2-yl)-ethenesulfonic Acid [1-(4-imidazol-1-yl-benzoyl)-pyrrolidin-3-ylmethyl]-amide

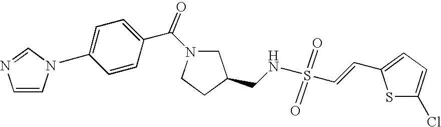

Following a procedure analogous to that described in Example 1, the title compound was obtained. MS found: (M+1)⁺=477.

### Example 25

### (S)-2-(5-Chloro-thiophen-2-yl)-ethenesulfonic Acid {1-[1-(2-methyl-pyrimidin-4-yl)-piperidine-4-carbonyl]-pyrrolidin-3-ylmethyl}-amide

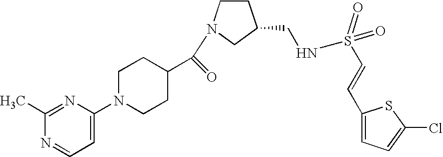

Following a procedure analogous to that described in Example 1, the title compound was obtained. MS found: (M+1)⁺=5 10.

### Example 26

### (S)-2-(5-Chloro-thiophen-2-yl)-ethenesulfonic Acid {1-[1-(6-fluoro-quinolin-4-yl)-piperidine-4-carbonyl]-pyrrolidin-3-ylmethyl}-amide

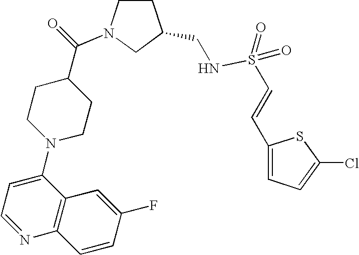

Following a procedure analogous to that described in Example 1, the title compound was obtained. MS found: (M+1)⁺=563.

### Example 27

### 2-(5-Chloro-thiophen-2-yl)-ethenesulfonic Acid {1-[2-oxo-2-(2S-pyrrolidin-1-ylmethyl-pyrrolidin-1-yl)-acetyl]-pyrrolidin-3S-ylmethyl}-amide

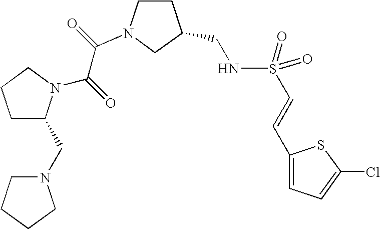

Following a procedure analogous to that described in Example 1, the title compound was obtained. MS found: (M+1)⁺=515.

### Example 28

### 2-(5-Chloro-thiophen-2-yl)-ethenesulfonic Acid {1-[2-oxo-2-(2S-pyrrolidin-1-ylmethyl-pyrrolidin-1-yl)-acetyl]-pyrrolidin-3R-ylmethyl}-amide

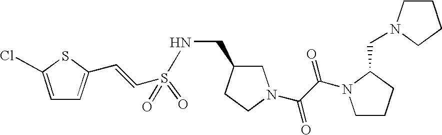

Following a procedure analogous to that described in Example 1, the title compound was obtained. MS found: (M+1)⁺=515.

### Example 29

### (R)-2-(5-Chloro-thiophen-2-yl)-ethenesulfonic Acid {1-[1-(2-methyl-pyrimidin-4-yl)-piperidine-4-carbonyl]-pyrrolidin-3-ylmethyl}-amide

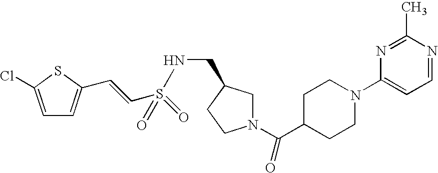

Following a procedure analogous to that described in Example 1, the title compound was obtained. MS found: (M+1)⁺=510.

### Example 30

### (S)-2-(5-Chloro-thiophen-2-yl)-ethenesulfonic Acid {1-[2-(2-dimethylamino-methyl-piperidin-1-yl)-2-oxo-acetyl]-pyrrolidin-3-ylmethyl}-amide

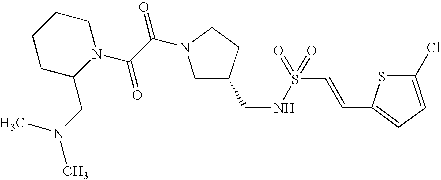

Following a procedure analogous to that described in Example 1, the title compound was obtained. MS found: (M+1)⁺=503.

### Example 31

### (R)-5′-Chloro-[2,2′]bithiophenyl-5-sulfonic Acid [1-(3,4,5,6-tetrahydro-2H-[1,4′]bipyridinyl-4-carbonyl)-pyrrolidin-3-ylmethyl]-amide

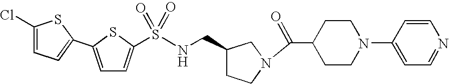

Following a procedure analogous to that described in Example 1, the title compound was obtained. MS found: (M+1)⁺=551.

### Example 32

### (R)-5′-Chloro-[2,2′]bithiophenyl-5-sulfonic Acid [1-(4-imidazol-1-yl-benzoyl)-pyrrolidin-3-ylmethyl]-amide

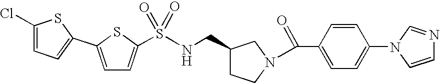

Following a procedure analogous to that described in Example 1, the title compound was obtained. MS found: (M+1)⁺=533.

### Example 33

### (R)-5′-Chloro-[2,2′]bithiophenyl-5-sulfonic Acid {1-[1-(2-methyl-pyrimidin-4-yl)-piperidine-4-carbonyl]-pyrrolidin-3-ylmethyl}-amide

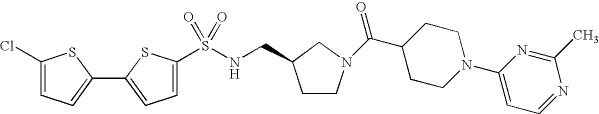

Following a procedure analogous to that described in Example 1, the title compound was obtained. MS found: (M+1)⁺=566.

### Example 34

### 5′-Chloro-[2,2′]bithiophenyl-5-sulfonic Acid [1-(2′-methanesulfonyl-biphenyl-4-carbonyl)-pyrrolidin-3-yl]-amide

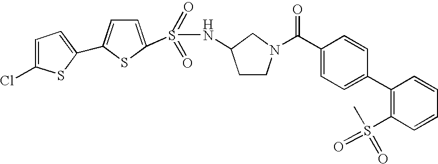

Following a procedure analogous to that described in Example 1, the title compound was obtained as a white solid. MS found: (M+1)⁺=607.

### Example 35

### N-{4-[1-(2′-Methanesulfonyl-biphenyl-4-carbonyl)-pyrrolidin-3-ylsulfamoyl]-phenyl}-acetamide

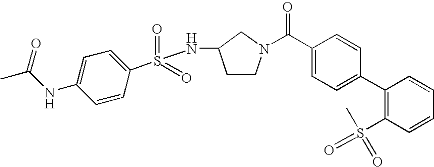

Following a procedure analogous to that described in Example 1, the title compound was obtained as a yellow solid. MS found: (M+1)⁺=542.

### Example 36

### 5-Isoxazol-3-yl-thiophene-2-sulfonic Acid [1-(2′-methanesulfonyl-biphenyl-4-carbonyl)-pyrrolidin-3-yl]-amide

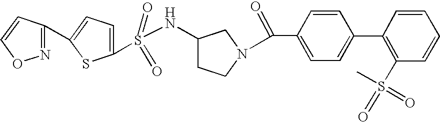

Following a procedure analogous to that described in Example 1, the title compound was obtained as a yellow solid. MS found: (M+1)⁺=558.

### Example 37

### 5-(1-Methyl-5-trifluoromethyl-1H-pyrazol-3-yl)-thiophene-2-sulfonic Acid [1-(2′-methanesulfonyl-biphenyl-4-carbonyl)-pyrrolidin-3-yl]-amide

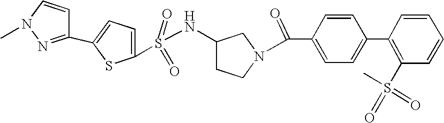

Following a procedure analogous to that described in Example 1, the title compound was obtained as a white solid. MS found: (M+1)⁺=639.

### Example 38

### 6-Chloro-thieno[2,3-b]pyridine-2-sulfonic Acid [1-(2′-methanesulfonyl-biphenyl-4-carbonyl)-pyrrolidin-3-yl]-amide

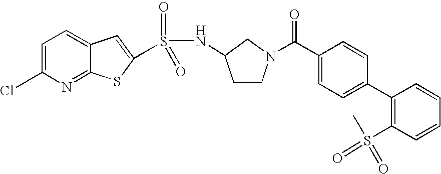

Following a procedure analogous to that described in Example 1, the title compound was obtained as a white solid. MS found: (M+1)⁺=576.

### Example 39

### 6-Chloro-naphthalene-2-sulfonic Acid [1-(2′-methanesulfonyl-biphenyl-4-carbonyl)-pyrrolidin-3-ylmethyl]-amide

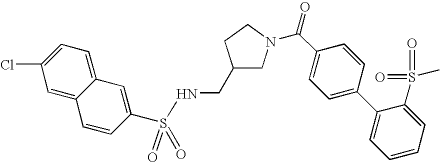

Following a procedure analogous to that described in Example 1, the title compound was obtained as a white solid. MS found: (M+1)⁺=583.

### Example 40

### 2-Methanesulfonyl-N-[1-(2′-methanesulfonyl-biphenyl-4-carbonyl)-pyrrolidin-3-ylmethyl]-benzenesulfonamide

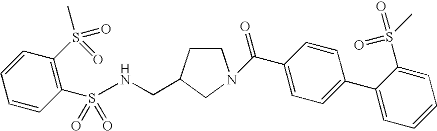

Following a procedure analogous to that described in Example 1, the title compound was obtained as a white solid. MS found: (M+1)⁺=576.

### Example 41

### N-[1-(2′-Methanesulfonyl-biphenyl-4-carbonyl)-pyrrolidin-3-ylmethyl-1-4-trifluoromethyl-benzenesulfonamide

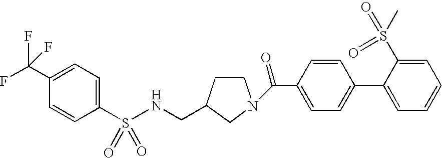

Following a procedure analogous to that described in Example 1, the title compound was obtained as a white solid. MS found: (M+1)⁺=566.

### Example 42

### 5-Isoxazol-3-yl-thiophene-2-sulfonic Acid [1-(2′-methanesulfonyl-biphenyl-4-carbonyl)-pyrrolidin-3-ylmethyl]-amide

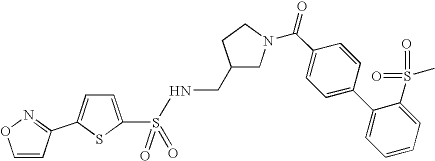

Following a procedure analogous to that described in Example 1, the title compound was obtained as a white solid. MS found: (M+1)⁺=571.

### Example 43

### 6-Chloro-3-methyl-benzo[b]thiophene-2-sulfonic Acid [1-(2′-methanesulfonyl-biphenyl-4-carbonyl)-pyrrolidin-3-yl]-amide

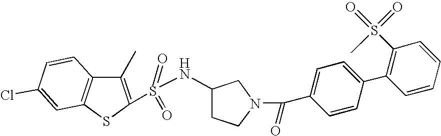

Following a procedure analogous to that described in Example 1, the title compound was obtained as a white solid. MS found: (M+1)⁺=589.

### Example 44

### 6-Chloro-3-methyl-benzo[b]thiophene-2-sulfonic Acid [1-(3,4,5,6-tetrahydro-2H-[1,4′]bipyridinyl-4-carbonyl)-pyrrolidin-3-yl]-amide

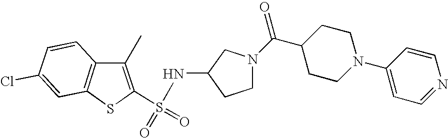

Following a procedure analogous to that described in Example 1, the title compound was obtained as a white solid. MS found: (M+1)⁺=525.

### Example 45

### 6-Bromo-naphthalene-2-sulfonic Acid [1-(3,4,5,6-tetrahydro-2H-[1,4′]bipyridinyl-4-carbonyl)-pyrrolidin-3-ylmethyl]-amide

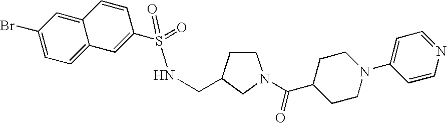

Following a procedure analogous to that described in Example 1, the title compound was obtained as a white solid. MS found: (M+1)⁺=583.

### Example 46

### 6-Chloro-naphthalene-2-sulfonic Acid [1-(3,4,5,6-tetrahydro-2H-[1,4′]bipyridinyl-4-carbonyl)-pyrrolidin-3-yl]-amide

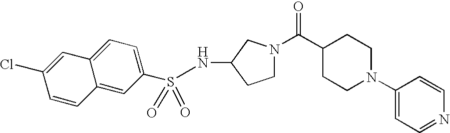

Following a procedure analogous to that described in Example 1, the title compound was obtained as a white solid. MS found: (M+1)⁺=499.

### Example 47

### 6-Bromo-naphthalene-2-sulfonic Acid [1-(3,4,5,6-tetrahydro-2H-[1,4′]bipyridinyl-4-carbonyl)-pyrrolidin-3-yl]-amide

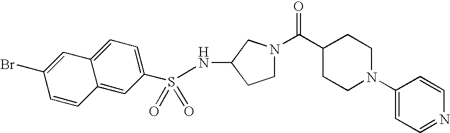

Following a procedure analogous to that described in Example 1, the title compound was obtained as a white solid. MS found: (M+1)⁺=543.

### Example 48

### 5-Chloro-3-methyl-benzo[b]thiophene-2-sulfonic Acid [1-(3,4,5,6-tetrahydro-2H-[1,4′]bipyridinyl-4-carbonyl)-pyrrolidin-3-ylmethyl]-amide

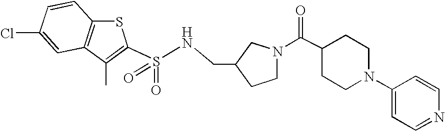

Following a procedure analogous to that described in Example 1, the title compound was obtained as a white solid. MS found: (M+1)⁺=533.

### Example 49

### 5-Chloro-benzo[b]thiophene-2-sulfonic Acid [1-(3,4,5,6-tetrahydro-2H-[1,4′]bipyridinyl-4-carbonyl)-pyrrolidin-3-yl]-amide

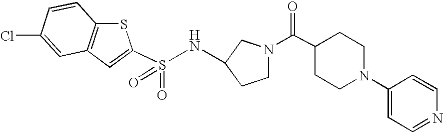

Following a procedure analogous to that described in Example 1, the title compound was obtained as a white solid. MS found: (M+1)⁺=505.

### Example 50

### 5′-Chloro-[2,2′]bithiophenyl-5-sulfonic Acid {1-[2-oxo-2-(2S-pyrrolidin-1-ylmethyl-pyrrolidin-1-yl)-acetyl]-pyrrolidin-3S-ylmethyl}-amide

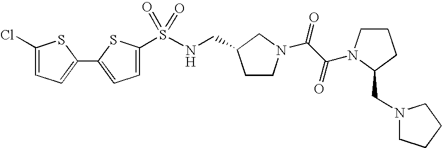

Following a procedure analogous to that described in Example 1, the title compound was obtained. MS found: (M+1)⁺=571.

### Example 51

### (S)-5′-Chloro-12,2′]bithiophenyl-5-sulfonic Acid [1-(2-oxo-2-pyrrolidin-1-yl-acetyl)-pyrrolidin-3-ylmethyl]-amide

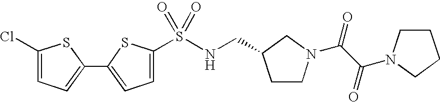

Following a procedure analogous to that described in Example 1, the title compound was obtained. MS found: (M+1)⁺=488.

### Example 52

### (R)-5′-Chloro-[2,2′]bithiophenyl-5-sulfonic Acid [1-(imidazole-1-carbonyl)-pyrrolidin-3-ylmethyl]-amide

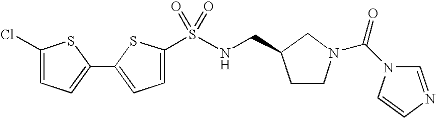

Following a procedure analogous to that described in Example 1, the title compound was obtained. MS found: (M+1)⁺=457.

### Example 53

### 4′-Chloro-biphenyl-3-sulfonic Acid [1-(2′-methanesulfonyl-biphenyl-4-carbonyl)-pyrrolidin-3-yl]-amide

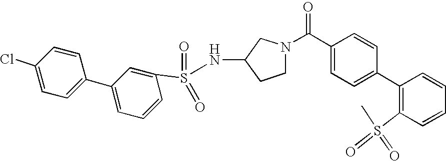

Following a procedure analogous to that described in Example 1, the title compound was obtained. MS found: (M+1)⁺=595.

### Example 54

### 5-Bromo-6-chloro-pyridine-2-sulfonic Acid [1-(2′-methanesulfonyl-biphenyl-4-carbonyl)-pyrrolidin-3-yl]-amide

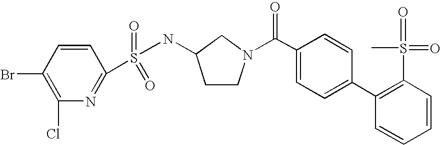

Following a procedure analogous to that described in Example 1, the title compound was obtained. MS found: (M+1)⁺=598.

### Example 55

### 3′,4′-Dichloro-biphenyl-4-sulfonic Acid [1-(2′-methanesulfonyl-biphenyl-4-carbonyl)-pyrrolidin-3-ylmethyl]-amide

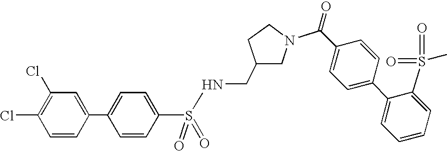

Following a procedure analogous to that described in Example 1, the title compound was obtained. MS found: (M+1)⁺=643.

### Example 56

### 5-Pyridin-2-yl-thiophene-2-sulfonic Acid [1-(2′-methanesulfonyl-biphenyl-4-carbonyl)-pyrrolidin-3-ylmethyl]-amide

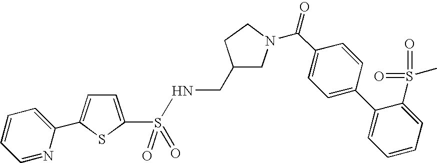

Following a procedure analogous to that described in Example 1, the title compound was obtained. MS found: (M+1)⁺=582.

### Example 57

### 5-Isoxazol-3-yl-thiophene-2-sulfonic Acid [1-(2′-methanesulfonyl-biphenyl-4-carbonyl)-pyrrolidin-3-ylmethyl]-amide

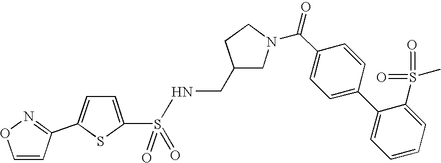

Following a procedure analogous to that described in Example 1, the title compound was obtained. MS found: (M+1)⁺=572.

### Example 58

### 6-Chloro-3-methyl-benzo[b]thiophene-2-sulfonic Acid [1-(2′-methanesulfonyl-biphenyl-4-carbonyl)-pyrrolidin-3-yl]-amide

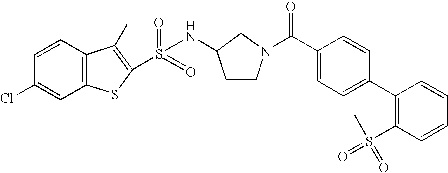

Following a procedure analogous to that described in Example 1, the title compound was obtained. MS found: (M+1)⁺=589.

### Example 59

### Benzo[1,2,5]oxadiazole-4-sulfonic Acid [1-(2′-methanesulfonyl-biphenyl-4-carbonyl)-pyrrolidin-3-yl]-amide

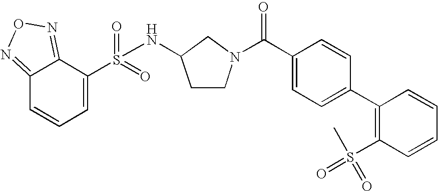

Following a procedure analogous to that described in Example 1, the title compound was obtained. MS found: (M+1)⁺=527.

### Example 60

### Benzo[d]isoxazole-5-sulfonic Acid [1-(2′-methanesulfonyl-biphenyl-4-carbonyl)-pyrrolidin-3-yl]-amide

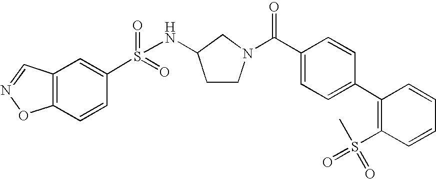

Following a procedure analogous to that described in Example 1, the title compound was obtained. MS found: (M+1)⁺=502.

### Example 61

### 2-Amino-pyrimidine-5-sulfonic Acid [1-(2′-methanesulfonyl-biphenyl-4-carbonyl)-pyrrolidin-3-yl]-amide

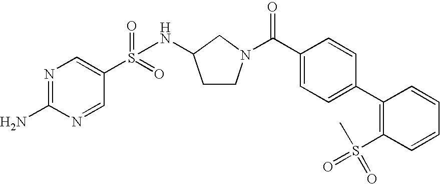

Following a procedure analogous to that described in Example 1, the title compound was obtained. MS found: (M+1)⁺=589.

### Example 62

### 2-Oxo-2,3-dihydro-1H-indole-5-sulfonic Acid [1-(2′-methanesulfonyl-biphenyl-4-carbonyl)-pyrrolidin-3-yl]-amide

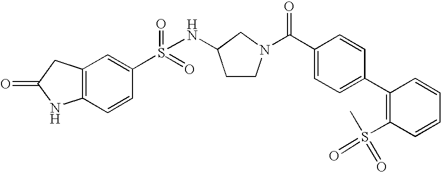

Following a procedure analogous to that described in Example 1, the title compound was obtained. MS found: (M+1)⁺=540.

### Example 63

### 6-Chloro-3-methyl-benzo[b]thiophene-2-sulfonic Acid [1-(3,4,5,6-tetrahydro-2H-[1,4′]bipyridinyl-4-carbonyl)-pyrrolidin-3-yl]-amide

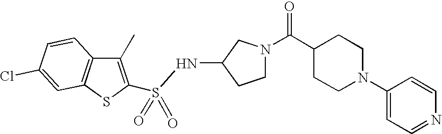

Following a procedure analogous to that described in Example 1, the title compound was obtained. MS found: (M+1)⁺=519.

### Example 64

### N-(1-(2-Chloro-1H-indole-6-carbonyl)piperidin-3-yl)-4-(2-oxopyridin-1(2H)-yl)benzamide

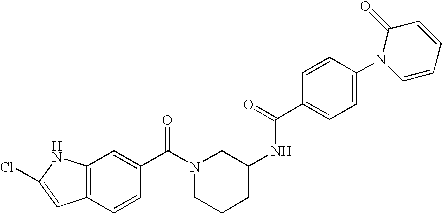

Part A (Preparation of methyl 4-(2-oxopyridin-1(2H)-yl)benzoate): In a three necked round bottom flask (500 mL) were mixed methyl 4-iodo benzoate (30 g, 114.5 mmol), 2-hydroxy pyridine (16.33 g, 171.7 mmol, 1.5 equivalent), K₂CO₃ (47.47 g, 343.5 mmol, 3 eq), CuI (10.91 g, 57.3 mmol, 0.5 eq), 1,10-phenanthroline (10.32 g, 57.3 mmol, 0.5 eq), and 230 mL of anhydrous DMSO. The dark suspension was heated to 110° C. for 3h under N₂ atmosphere. The mixture was filtered to eliminate most of K₂CO₃. The filtrate was washed abundantly with CH₂Cl₂, and the obtained organic layer was washed with water (3×30 mL), dried over Na₂SO₄, and concentrated to yield 21 g of the crude material, which was pure enough to continue the reaction sequence. An analytically pure sample was obtained after recrystallization in MeOH. ¹H NMR (400 MHz, DMSO-d₆) δ 8.07 (d, J=8.6 Hz, 2H), 7.68 (d, 1H), 7.59 (d, J=8.6 Hz, 2H), 7.5 (m, 1H), 6.51 (d, J=9.4 Hz, 1H) 6.36 (t, J=6.7 Hz, 1H), 3.89 (s, 3H), ppm. LC-MS (ESI) 230.17 [M+H]⁺, t_{R}^{=1.903} min (Method A: HPLC: Shimadzu Phenomenex Luna C18 4.6×50 mm; flow rate 4 ml/mn; detection at 220 nM; Gradient elution 0% to 100% B over 4 min; (A=10% MeOH, 90% H₂O, 0.1% TFA & B=90% MeOH, 10% H₂O, 0.1% TFA).

Part B (Preparation of 4-(2-oxopyridin-1(2H)-yl)benzoic acid): The ester prepared in part A (18 g, 0.079 mol) was mixed with MeOH (250 mL) and 1N NaOH (120 mL, 1.5 eq). The reaction mixture was stirred for 3h at room temperature. The initial suspension went completely into solution by the end of the reaction. The methanol was evaporated, and the obtained solution extracted with CH₂Cl₂ (3×30 mL). The aqueous solution was acidified with 1N HCl (pH=2-3). The white precipitate was collected by filtration and washed with 0.1N HCl, water, and MeOH to yield the target acid as a white solid (16 g). ¹H NMR (400 MHz, DMSO-d₆) δ 8.05 (d, J=8.4 Hz, 2H), 7.68 (d, 1H), 7.55 (d, J=8.4 Hz, 2H), 7.53 (m, 1H), 6.51 (d, 1H) 6.36 (t, 1H), ppm.

Part C (Preparation of 4-(2-oxopyridin-1(2H)-yl)benzoyl chloride): The acid prepared in part B (215.2 mg, 1 mmol) was refluxed for 1 h with SOCl₂ (5 mL). Volatiles were evaporated to yield the expected acid chloride as an off-white solid, which was used in the next step without further purification.

Part D (Preparation of tert-butyl 3-(4-(2-oxopyridin-1(2H)-yl)benzamido)piperidine-1-carboxylate): Commercial racemic 3-amino-1-N-Boc piperidine (100 mg, 0.5 mmol) was mixed with anhydrous CH₂Cl₂ (2 mL), TEA (100 μL), and the acid chloride (116.8 mg, 0.5 mmol) prepared in part C. The mixture was stirred overnight at room temperature and washed with water (2×1 mL). The organic phase was dried over Na₂SO₄ and evaporated to yield the expected compound as a crude yellow solid (207.2 mg), which was used without further purification in the next step of the reaction.

Part E (Preparation of tert-butyl 3-(4-(2-oxopyridin-1(2H)-yl)benzamido)piperidine-1-carboxylate trifluoroacetic acid salt): To the N-Boc protected compound from step D above (57 mg, 0.143 mmol), dissolved in anhydrous CH₂Cl₂ (1 mL), was added trifluoroacetic acid (1 mL). The mixture was stirred at room temperature overnight. The volatiles were evaporated to yield the free amine as a TFA salt, which was used in the next step without further purification.

Part F (Preparation of N-(1-(2-chloro-1H-indole-6-carbonyl)piperidin-3-yl)-4-(2-oxopyridin-1(2H)-yl)benzamide): The free amine prepared in step E (40.7 mg, 0.099 mmol) was mixed with anhydrous CH₂Cl₂ (2 mL) and 100 μL of TEA. The mixture was sonicated for complete solubilization and then 2-chloro-1H-indole-6-carbonyl chloride (30 mg, 0.14 mmol, 1.4 eq)(freshly prepared from the corresponding acid according to the procedure of step C) was added, and the mixture stirred overnight. The precipitate was filtered, washed with CH₂Cl₂ and dried to yield crude material (34.8 mg). An analytically pure sample was prepared after purification by prep HPLC. (Method B: Shimadzu Phenomenex Luna 5u 21.2×100; flow rate 20 ml/min; detection at 220 nM; Gradient elution 10% to 100% B over 15 min; (A=10% MeOH, 90% H₂O, 0.1% TFA& B=90% MeOH, 10% H₂O, 0.1% TFA). ¹H NMR MHz, MeOD) δ 8.13 (s, 1H), 7.8-8 (m, 2H), 7.78 (m, 1H), 7.45-7.65 (m, 6H), 7.37 (d, J=4 Hz, 1H), 7.19 (d, J=8 Hz, 1H), 6.65 (d, J=9.4 Hz, 1H), 6.50 (t, J=6.7 Hz, 1H), 4.55 (m, 1H), 3.65-4.3 (m, 4H), 1.6-2.2 (m, 4H), ppm. LC-MS (ESI) 475.15 [M+H]⁺, t_{R}^{=5.718} min (HPLC Method C: Shimadzu Phenomenex S5 ODS 4.6×50 mm Luna flow rate 2.5 ml/mn; detection at 220 nM; Gradient elution 0% to 100% B over 8 min; (A=10% MeOH, 90% H₂O, 0.2% H₃PO₄ & B=90% MeOH, 10% H₂O, 0.2% H₃PO₄). HRMS (ESI) m/z calculated for C₂₆H₂₄ClN₄O₃ [M+H]⁺ 475.1537, found 475.1558.

According to the procedure described in step D and by the reaction of the appropriated amino compound with the corresponding acid chloride, the following intermediate compounds were prepared:

### tert-Butyl 3-(2-chlorothiophene-5-carboxamido)piperidine-1-carboxylate

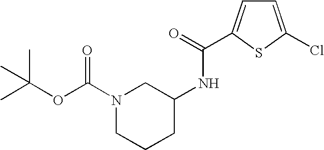

LC-MS (ESI) 367.13 [M+Na]⁺, t_{R}=3.467 min (HPLC Method A).

### tert-Butyl 1-(4-(2-oxopyridin-1(2H)-yl)benzoyl)pyrrolidin-3-ylcarbamate

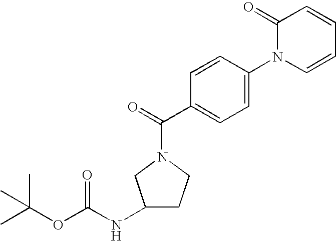

t_{R}=2.469 min (HPLC Method D: Shimadzu Phenomenex S5 ODS 4.6×50 mm Luna flow rate 2.5 ml/mn; detection at 220 nM; Gradient elution 0% to 100% B over 4 min; (A=10% MeOH, 90% H₂O, 0.2% H3PO4& B=90% MeOH, 10% H₂O, 0.2%).

### tert-Butyl 1-(2-chlorothiophene-5-carbonyl)pyrrolidin-3-ylcarbamate

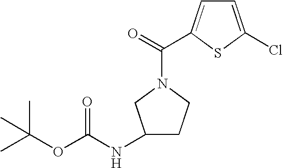

LC-MS (ESI) 353.12 [M+Na]⁺, t_{R}=3.237 min (HPLC Method A).

### tert-Butyl 3-(3-chloro-1H-indole-6-carboxamido)piperidine-1-carboxylate

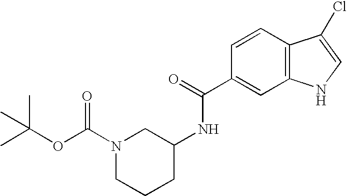

LC-MS (ESI) 400.17 [M+Na]⁺, t_{R}=3.477 min (HPLC Method A).

### tert-Butyl 1-(3-chloro-1H-indole-6-carbonyl)pyrrolidin-3-ylcarbamate

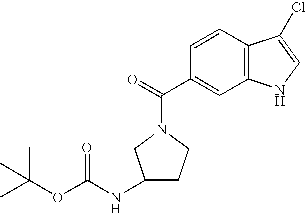

LC-MS (ESI) 364.18 [M+H]⁺, t_{R}=3.363 min (HPLC Method A).

According to the procedure of step E followed by the procedure of step F and by the reaction of the appropriated acid chlorides with the corresponding deprotected amino derivatives, the examples 65 to 69 were prepared:

### Example 65

### 3-chloro-N-(1-(4-(2-oxopyridin-1(2H)-yl)benzoyl)pyrrolidin-3-yl)-1H-indole-6-carboxamide

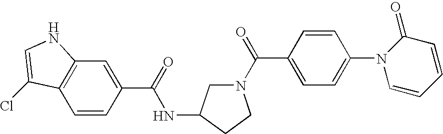

The title compound was purified first by liquid chromatography (ISCO system; 4 g cartridge, Eluent MeOH/CH₂Cl₂ gradient elution 0% to 10%) then by prep HPLC (Method B). ¹H NMR (500 MHz, MeOD) δ 7.96(s, 0.5H), 7.89(s, 0.5H), 7.72 (m, 2H), 7.48-7.66 (m, 6H), 7.41 (d, J=9.4 Hz, 1H), 6.64 (t, J=9 Hz, 1H), 6.5 (q, J=14.1 Hz, and J=6.7 Hz, 1H), 4.74 (t, 0.5H), 4.58 (t, 0.5H), 4 (q, 0.5H), 3.9 (m, 1H), 3.65-3.76 (m, 2H), 3.52 (m, 0.5H), 2.25-2.42 (m, 1H), 2.05-2.22 (m, 1H), ppm. LC-MS (ESI) 461.14 [M+H]⁺, t_{R}=5.412 min (HPLC Method C). HRMS (ESI) m/z calculated for C₂₅H₂₂ClN₄O₃ [M+H]⁺ 461.13805, found 461.1374.

### Example 66

### N-(1-(3-chloro-1H-indole-6-carbonyl)pyrrolidin-3-yl)-4-(2-oxopyridin-1(2H)-yl)benzamide

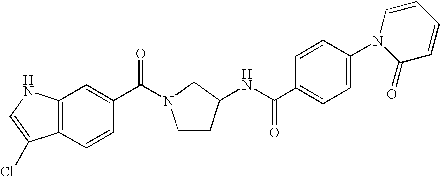

The title compound was purified by prep HPLC (Shimadzu Phenomenex Luna 5u 21.2×100; flow rate 20 ml/min; detection at 220 nM; Gradient elution 15% to 75% B over 15 min; (A=10% MeOH, 90% H₂O, 0.1% TFA& B=90% MeOH, 10% H₂O, 0.1% TFA). ¹H NMR (500 MHz, CDCl₃) δ 8 (d, J=8 Hz, 1H), 7.18-7.62(m, 9H), 6.83 (d, J=9.4 Hz, 1H), 6.49 (t, J=6.7 Hz, 1H), 4.59 (m, 1H), 3.4-4 (m, 4H), 2-2.2 (m, 2H), ppm. LC-MS (ESI) 461.14 [M+H]⁺, t_{R}=5.425 min (HPLC Method C). HRMS (ESI) m/z calculated for C₂₅H₂₂ClN₄O₃ [M+H]⁺ 461.13805, found 461.1387.

### Example 67

### 3-chloro-N-(1-(4-(2-oxopyridin-1(2H)-yl)benzoyl)piperidin-3-yl)-1H-indole-6-carboxamide

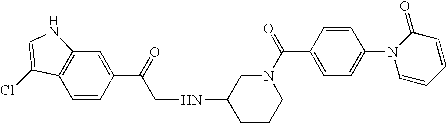

The title compound was purified by prep HPLC (Shimadzu Phenomenex Luna 5u 21.2×100; flow rate 20 ml/min; detection at 220 nM; Gradient elution 10% to 85% B over 15 min; (A=10% MeOH, 90% H₂O, 0.1% TFA& B=90% MeOH, 10% H₂O, 0.1% TFA). ¹H NMR (500 MHz, CDCl₃) δ 7.2-7.9 (m, 10H), 6.78 (d, J=9.4 Hz, 1H), 6.4 (t, J=6.7 Hz, 1H), 3.4-4.2 (m, 5H), 1.5-2.1 (m, 4H), ppm. LC-MS (ESI) 475.15 [M+H]⁺, t_{R}=5.465 min (HPLC Method C) HRMS (ESI) m/z calculated for C₂₆H₂₄ClN₄O₃ [M+H]⁺475.1537, found 475.1526.

### Example 68

### 5-chloro-N-(1-(4-(2-oxopyridin-1(2H)-yl)benzoyl)piperidin-3-yl)thiophene-2-carboxamide

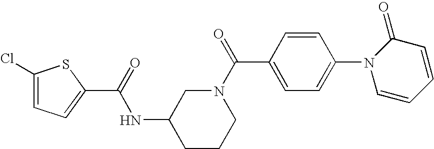

The title compound was purified by prep HPLC (Shimadzu Phenomenex Luna 5u 21.2×100; flow rate 20 m/min; detection at 220 nM; Gradient elution 15% to 80% B over 15 min; (A=10% MeOH, 90% H₂O, 0.1% TFA& B=90% MeOH, 10% H₂O, 0.1% TFA). ¹H NMR (400 MHz, CDCl₃) δ 9 (bs, 1H), 7.26-7.65 (m, 7H), 6.89 (bs, 1H), 6.83 (d, J=9.4 Hz, 1H), 6.44 (t, J=6.7 Hz, 1H), 3.1-4.2 (m, 5H), 1.56-2.11 (m, 4H), ppm. LC-MS (ESI) 442.1 [M+H]⁺, t_{R}=5.215 min (HPLC Method C). HRMS (ESI) m/z calculated for C₂₁H₁₉ClN₃O₃ [M+H]⁺428.08357, found 428.0836.

### Example 69

### N-(1-(2-chlorothiophene-5-carbonyl)pyrrolidin-3-yl)-4-(2-oxopyridin-1(2H)-yl)benzamide

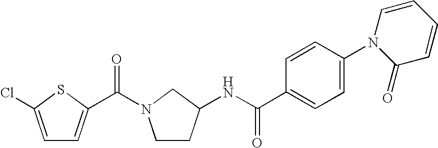

The title compound was purified by crystallization in MeOH. ¹H NMR

MHz, DMSO-d₆) δ 8.75 (m, 1H), 7.96 (m, 2H), 7.65 (d, J=6.7 Hz, 1H), 7.52 (m, 4H), 7.2 (s, 1H), 6.49 (d, J=9.4 Hz, 1H) 6.34 (t, J=6.7 Hz, 1H), 4.59 (m, 1H), 3.5-4.1 (m, 4H), 2.14 (m, 2H), ppm. LC-MS (ESI) 428.08 [M+H]⁺, t_{R}=5.352 min (HPLC Method C). HRMS (ESI) m/z calculated for C₂₁H₁₉ClN₃O₃ [M+H]⁺ 428.08357, found 428.0836.

### Example 70

### N-(4-chlorophenyl)-3-(4-(2-oxopyridin-1(2H)-yl)benzamido)pyrrolidine-1-carboxamide

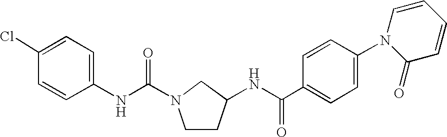

Part A (Preparation of tert-butyl 1-((4-chlorophenyl)carbamoyl)pyrrolidin-3-ylcarbamate): (±)-3-Amino-1-N-Boc pyrrolidine (93.2 mg, 0.5 mmol) was mixed with CH₂Cl₂ (2 mL) and TEA (100 μL). To this solution was added 4-chlorophenylisocyanate (1 eq). The mixture was stirred overnight at room temperature. CH₂Cl₂ (5 mL) was added, and the mixture washed with water (2×2 mL). The organic phase was dried over MgSO₄ and concentrated to yield the target compound (152.8 mg) as a white powder, which was used in the next step without further purification. LC-MS (ESI) 362.17 [M+Na]⁺, t_{R}=3.23 min (HPLC Method A).

Part B (Preparation of N-(4-chlorophenyl)-3-(4-(2-oxopyridin-1(2H)-yl)benzamido)pyrrolidine-1-carboxamide): tert-Butyl 1-((4-chlorophenyl)carbamoyl)pyrrolidin-3-ylcarbamate (56 mg, 0.165 mmol), prepared as above, was deprotected by reaction with TFA/CH₂Cl₂ according to the procedure of step E of Example 64. The volatiles were evaporated, and anhydrous CH₂Cl₂ (1 mL) added together with TEA (100 μL) were added. Next, the acid chloride prepared in step C Example 64 (1 eq) was introduced, and the mixture stirred at room temperature for two hours. CH₂Cl₂ (5 mL) were added, and the mixture washed with water (2×2 mL). The organic phase was dried over MgSO₄ and concentrated to yield the target compound (33.4 mg) as a white solid. ¹H NMR (400 MHz, DMSO-d₆) δ 8.73 (d, J=6.7 Hz, 1H), 8.33 (s, 1H), 7.98 (d, J=8.7 Hz, 2H), 7.67 (q, J=2 Hz and 6.7 Hz, 1H), 7.5-7.6 (m, 5H), 7.27 (d, J=8.7 Hz), 6.49 (d, J=9.4 Hz, 1H) 6.34 (t, J=6.7 Hz, 1H), 4.52 (m, 1H), 3.73 (m, 1H), 3.56 (m, 1H), 3.3-3.2 (m, 2H), 2.16 (m, 1H), 2 (m, 1H) ppm. LC-MS (ESI) 437.14 [M+H]⁺, t_{R}=5.205 min (HPLC Method C). HRMS (ESI) m/z calculated for C₂₄H₂₂ClN₄O₃ [M+H]⁺ 437.13805, found 475.1379.

Numerous modifications and variations of the present invention are possible in light of the above teachings. It is therefore to be understood that within the scope of the appended claims, the invention may be practiced otherwise that as specifically described herein.

1. A compound of formula Ia or Ib: 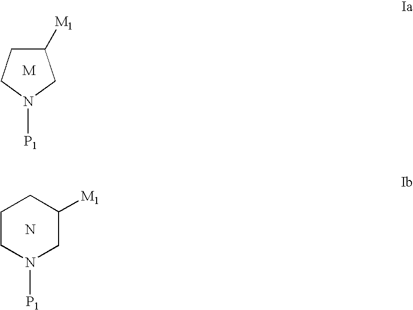
or a stereoisomer or pharmaceutically acceptable salt thereof, wherein;
rings M and N are substituted with 0-3 R^{1a}, 0-2 R³, and 0-1 double bonds;
one of P₁ and M₁ is -Z-A-B and the other -G₁-G;
G is a group of formula IIa or IIb: 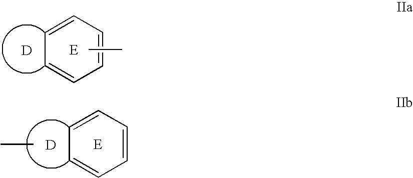
ring D, including the two atoms of ring E to which it is attached, is a 5-6 membered ring consisting of: carbon atoms and 0-2 heteroatoms selected from the group consisting of N, O, and S(O)ₚ;
ring D is substituted with 0-2 R and there are 0-3 ring double bonds;
E is selected from phenyl, pyridyl, pyrimidyl, pyrazinyl, and pyridazinyl, and is substituted with 1-3 R;
alternatively, ring D is absent, and ring E is selected from phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1-3 R;
alternatively, ring D is absent, ring E is selected from phenyl, phenyl, pyridyl, pyrimidyl, pyrazinyl, pyridazinyl, pyrrolyl, pyrazolyl, imidazolyl, isoxazolyl, oxazolyl, triazolyl, thienyl, and thiazolyl, and ring E is substituted with 1 R and either phenyl or a 5-6 membered heterocycle consisting of: carbon atoms and 1-4 heteroatoms selected from the group consisting of N, O, and S(O)ₚ, wherein the phenyl is substituted with 1-2 R and the 5-6 membered heterocycle is substituted with 0-2 carbonyls and 1-2 R and has 0-3 ring double bonds;
R is selected from H, C₁₋₄ alkyl, F, Cl, Br, I, OH, OCH₃, OCH₂CH₃, OCH(CH₃)₂, OCH₂CH₂CH₃, CN, C(═NR⁸)NR⁷R⁹, NHC(═NR⁸)NR⁷R⁹, NR⁸CH(═NR⁷), NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, CH₂CH₂NH₂, CH₂CH₂NH(C₁₋₃ alkyl), CH₂CH₂N(C₁₋₃ alkyl)₂, (CR⁸R⁹)ₜC(O)H, (CR⁸R⁹)ₜC(O)R^{2c}, (CR⁸R⁹)ₜNR⁷R⁸, (CR⁸R⁹)ₜC(O)NR⁷R⁸, (CR⁸R⁹)ₜNR⁷C(O)R⁷, (CR⁸R⁹)ₜOR³, (CR⁸R⁹)ₜS(O)ₚNR⁷R⁸, (CR⁸R⁹)ₜNR⁷S(O)ₚR⁷, (CR⁸R⁹)ₜSR³, (CR⁸R⁹)ₜS(O)R³, (CR⁸R⁹)ₜS(O)₂R³, and OCF₃;
alternatively, when 2 R groups are attached to adjacent atoms, they combine to form methylenedioxy or ethylenedioxy;
A is selected from:
C₃₋₁₀ carbocycle substituted with 0-2 R⁴, and
5-12 membered heterocycle substituted with 0-2 R⁴ and consisting of: carbon atoms and 1-4 heteroatoms selected from the group consisting of N, O, and S(O)ₚ;
B is selected from: Y, X—Y, (CH₂)₀₋₂C(O)NR²R^{2a}, and (CH₂)₀₋₂NR²R^{2a}, provided that Z and B are attached to different atoms on A;
X is selected from —CR²R^{2a})₁₋₄—, —CR²(CR²R^{2b})(CH₂)ₜ—, —C(O)—, —C(═NR^{1b}—, CR²(NR^{1b}R²)—, —CR²(OR²)—, —CR²(SR²)—, —C(O)CR²R^{2a}—, —CR²R^{2a}C(O), —S(O)—, —S(O)₂—, SCR²R^{2a}—, S(O)CR²R^{2a}—, —S(O)₂CR²R^{2a}—, —CR²R^{2a}S—, —CR²R^{2a}S(O)—, —CR²R^{2a}S(O)₂—, —S(O)₂NR²—, —S(O)₂NR²CR²R^{2a}—, —CR²R^{2a}S(O)₂NR²—, —NR²S(O)₂—, —CR²R^{2a}NR²S(O)₂—, —NR²S(O)₂CR²R^{2a}—, —NR²C(O)—, —C(O)NR²—, —NR²C(O)CR²R^{2a}—, —C(O)NR²CR²R^{2a}—, —CR²R^{2a}NR²C(O)—, —CR²R^{2a}C(O)NR²—, NR², —NR²CR²R^{2a}—, CR²R^{2a}NR²—, O, —OCR²R^{2a}—, and —CR²R^{2a}O—;
Y is selected from:
C₃₋₁₀ carbocycle substituted with 0-2 R^{4a}, and
5-10 membered heterocycle consisting of: carbon atoms and 1-4 heteroatoms selected from the group consisting of N, O, and S(O)ₚ substituted with 0-2 R^{4a};
G₁ is selected from ═O or a 2-6 membered linear chain consisting of: 0-6 carbon atoms, 0-2 carbonyl groups, and 0-3 heteroatoms selected from O, N, and S(O)ₚ, and G₁ is substituted with 0-4 R³, and there are 0-2 double bonds and 0-1 triple bond; provided that other than an S—S, S—O, or O—O bond is present in G₁;
Z is selected from —CR³R^{3e})₁₋₄—, (CR³R^{3e})_{q}O(CR³R^{3e})_{q1}, (CR³R^{3e})_{q}NR^{3b}(CR³R^{3e})_{q1}, (CR³R^{3e})_{q}C(O)(CR³R^{3e})_{q1}, (CR³R^{3e})_{q}C(O)O(CR³R^{3e})_{q1}, (CR³R^{3e})_{q}OC(O)(CR³R^{3e})_{q1}, (CR³R^{3e})_{q}C(O)NR^{3b}(CR³R^{3e})_{q1}, (CR³R^{3e})_{q}NR^{3b}C(O)(CR³R^{3e})_{q1}, (CR³R^{3e})_{q}OC(O)O(CR³R^{3e})_{q1}, (CR³R^{3e})_{q}OC(O)NR^{3b}(CR³R^{3e})_{q1}, (CR³R^{3e})_{q}NR^{3b}C(O)O(CR³R^{3e})_{q1}, (CR³R^{3e})_{q}NR^{3b}C(O)NR^{3b}(CR³R^{3e})_{q1}, (CR³R^{3e})_{q}C(O)(CR³R^{3e})_{q}C(O)(CR³R^{3e})_{q1}, (CR³R^{3e})_{q}NR^{3b}(CR³R^{3e})_{q}C(O)NR^{3b}(CR³R^{3e})_{q1}, (CR³R^{3e})_{q}NR^{3b}C(O)(CR³R^{3e})_{q}C(O)(CR³R^{3e})_{q1}, (CR³R^{3e})_{q}C(O)(CR³R^{3e})_{q}C(O)NR^{3b}(CR³R^{3e})_{q1}, (CR³R^{3e})_{q}NR^{3b}C(O)(CR³R^{3e})_{q}C(O)NR^{3b}(CR³R^{3e})_{q1}, (CR³R^{3e})_{q}S(CR³R^{3e})_{q1}, (CR³R^{3e})_{q}S(O)(CR³R^{3e})_{q1}, (CR³R^{3e})_{q}S(O)₂(CR³R^{3e})_{q1}, (CR³R^{3e})_{q}SO₂NR^{3b}(CR³R^{3e})_{q1}, (CR³R^{3e})_{q}NR^{3b}SO₂(CR³R^{3e})_{q1}, (CR³R^{3e})_{q}S(O)NR^{3b}C(O)(CR³R^{3e})_{q1}, (CR³R^{3e})_{q}C(O)NR^{3b}S(O)₂(CR³R^{3e})_{q1}, and (CR³R^{3e})_{q}NR^{3b}SO₂NR^{3b}(CR³R^{3e})_{q1}, wherein q+q1 total 0, 1, 2, 3, or 4, provided that Z does not form a N—S, NCH₂N, NCH₂O, or NCH₂S bond with either group to which it is attached;
R^{1a}, at each occurrence, is selected from H, —(CR³R^{3a})ᵣ—R^{1b}, —(CR³R^{3a})ᵣ—CR³R^{1b}R^{1b}, —(CR³R^{3a})ᵣ—O—(CR³R^{3a})ᵣ—R^{1b}, —(CR³R^{3a})ᵣ—NR²—(CR³R^{3a})ᵣ—R^{1b}, —(CR³R^{3a})ᵣ—S(O)P—(CR³R^{3a})ᵣ—R^{1b}, —(CR³R^{3a})ᵣ—CO₂—(CR³R^{3a})ᵣ—R^{1b}, —(CR³R^{3a})ᵣ—C(O)NR²—(CR³R^{3a})ᵣ—R^{1b}, —(CR³R^{3a})ᵣ—C(O)—(CR³R^{3a})ᵣ—R^{1b}, —C₂₋₆ alkenylene-R^{1b}, —C₂₋₆ alkynylene-R^{1b}, and —(CR³R^{3a})ᵣ—C(═NR^{1b})NR³R^{1b}, provided that R^{1a} forms other than an N-halo, N—S, O—O, or N—CN bond;
provided that R^{1a} is other than a substituted or unsubstituted 3,4-dihydroxyphenyl group;
alternatively, when two R^{1a} groups are attached to adjacent atoms or to the same carbon atom, together with the atoms to which they are attached, they form a 5-7 membered ring consisting of: carbon atoms and 0-2 heteroatoms selected from the group consisting of N, O, and S(O)ₚ, this ring being substituted with 0-2 R^{4b} and comprising: 0-3 ring double bonds;
R^{1b} is selected from H, C₁₋₃ alkyl, F, Cl, Br, I, —CN, —NO₂, (CF₂)ᵣCF₃, (CR³R^{3a})ᵣOR², NR²R^{2a}, C(O)R^{2b}, CO₂R^{2b}, OC(O)R², CH(CH₂OR²)₂, (CF₂)ᵣCO₂R^{2a}, S(O)ₚR^{2b}, NR²(CH₂)ᵣOR², C(═NR^{2c})NR²R^{2a}, NR²C(O)R^{2b}, NR²C(O)NR²R^{2a}, NR²C(O)₂R^{2a}, OC(O)NR²R^{2a}, C(O)NR²R^{2a}, C(O)NR²(CH₂)ᵣOR², SO₂NR²R^{2a}, NR²SO₂R², C(O)NR²SO₂R², C₃₋₆ carbocycle substituted with 0-2 R^{4b}, and 5-10 membered heterocycle substituted with 0-2 R^{4b} and consisting of carbon atoms and from 1-4 heteroatoms selected from the group consisting of N, O, and S(O)ₚ, provided that R^{1b} forms other than an O—O, N-halo, N—S, or N—CN bond and provided that S(O)ₚR² forms other than S(O)₂H or S(O)H;
R², at each occurrence, is selected from H, CF₃, C₁₋₆ alkyl, benzyl, —(CH₂)ᵣ—C₃₋₁₀ carbocycle substituted with 0-2 R^{4b}, and —(CH₂)ᵣ-5-10 membered heterocycle substituted with 0-2 R^{4b} and consisting of: carbon atoms and 1-4 heteroatoms selected from the group consisting of N, O, and S(O)ₚ;
R^{2a}, at each occurrence, is selected from H, CF₃, C₁₋₆ alkyl, benzyl, —(CH₂)ᵣ—C₃₋₁₀ carbocycle substituted with 0-2 R^{4b}, and —(CH₂)ᵣ-5-10 membered heterocycle substituted with 0-2 R^{4b} and consisting of: carbon atoms and 1-4 heteroatoms selected from the group consisting of N, O, and S(O)ₚ;
alternatively, NR²R^{2a} forms a 5 or 6 membered saturated, partially saturated or unsaturated ring substituted with 0-2 R^{4b} and consisting of: 0-1 additional heteroatoms selected from the group consisting of N, O, and S(O)ₚ;
R^{2b}, at each occurrence, is selected from CF₃, C₁₋₄ alkoxy substituted with 0-2 R^{4b}, C₁₋₆ alkyl substituted with 0-2 R^{4b}, —(CH₂)ᵣ—C₃₋₁₀ carbocycle substituted with 0-2 R^{4b}, and —(CH₂)ᵣ-5-10 membered heterocycle substituted with 0-2 R^{4b} and consisting of: carbon atoms and 1-4 heteroatoms selected from the group consisting of N, O, and S(O)ₚ;
R^{2c}, at each occurrence, is selected from CF₃, OH, C₁₋₄ alkoxy, C₁₋₆ alkyl, —(CH₂)ᵣ C₃₋₁₀ carbocycle substituted with 0-2 R^{4b}, and —(CH₂)ᵣ-5-10 membered heterocycle substituted with 0-2 R^{4b} and consisting of carbon atoms and from 1-4 heteroatoms selected from the group consisting of N, O, and S(O)ₚ;
R³, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
R^{3a}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
alternatively, NR³R^{3a} forms a 5 or 6 membered saturated, partially unsaturated, or unsaturated ring consisting of: carbon atoms, the nitrogen atom to which R³ and R^{3a} are attached, and 0-1 additional heteroatoms selected from the group consisting of N, O, and S(O)ₚ;
R^{3b}, at each occurrence, is selected from H, C₁₋₆ alkyl substituted with 0-2 R^{1a}, C₂₋₆ alkenyl substituted with 0-2 R^{1a}, C₂₋₆ alkynyl substituted with 0-2 R^{1a}, —(C₀₋₄ alkyl)-5-10 membered carbocycle substituted with 0-3 R^{1a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;
R^{3c}, at each occurrence, is selected from CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, and phenyl;
R^{3d}, at each occurrence, is selected from H, CH₃, CH₂CH₃, CH₂CH₂CH₃, CH(CH₃)₂, CH₂CH₂CH₂CH₃, CH₂CH(CH₃)₂, CH(CH₃)CH₂CH₃, C₁₋₄ alkyl-phenyl, and C(═O)R^{3c};
R^{3e}, at each occurrence, is selected from H, S(O)₂NHR³, C(O)R³, C(O)NHR³, C(O)OR^{3f}, S(O)R^{3f}, S(O)₂R^{3f}, C₁₋₆ alkyl substituted with 0-2 R^{1a}, C₂₋₆ alkenyl substituted with 0-2 R^{1a}, C₂₋₆ alkynyl substituted with 0-2 R^{1a}, —(C₀₋₄ alkyl)-5-10 membered carbocycle substituted with 0-3 R^{1a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;
R^{3f}, at each occurrence, is selected from: C₁₋₆ alkyl substituted with 0-2 R^{1a}, C₂₋₆ alkenyl substituted with 0-2 R^{1a}, C₂₋₆ alkynyl substituted with 0-2 R^{1a}, —(C₀₋₄ alkyl)-5-10 membered carbocycle substituted with 0-3 R^{1a}, and —(C₀₋₄ alkyl)-5-10 membered heterocycle substituted with 0-3 R^{1a} and consisting of: carbon atoms and 1-4 heteroatoms selected from the group consisting of N, O, and S(O)ₚ;
R⁴, at each occurrence, is selected from H, ═O, (CR³R^{3a})ᵣOR², F, Cl, Br, I, C₁₋₄ alkyl, (CR³R^{3a})ᵣCN, (CR³R^{3a})ᵣNO₂, (CR³R^{3a})ᵣNR²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣNR²C(O)NR²R^{2a}, (CR³R^{3a})ᵣC(═NR²)NR²R^{2a}, (CR³R^{3a})ᵣC(═NS(O)₂R^{5a})NR²R^{2a}, (CR³R^{3a})ᵣNR²C(═NR²)NR²R^{2a}, (CR³R^{3a})ᵣC(O)NR²C(═NR²)NR²R^{2a}, (CR³R^{3a})ᵣSO₂NR²R^{2a}, (CR³R^{3a})ᵣNR²SO₂NR²R^{2a}, (CR³R^{3a})ᵣNR²SO₂—C₁₋₄ alkyl, (CR³R^{3a})ᵣNR²SO₂R^{5a}, (CR³R^{3a})ᵣS(O)ₚR^{5a}, (CR³R^{3a})ᵣ(CF₂)ᵣCF₃, N(CH₂)ᵣR^{1b}, O(CH₂)ᵣR b, S(CH₂)ᵣR^{1b}, (CR³R^{3a})ᵣ-5-6 membered carbocycle substituted with 0-1 R⁵, and a (CR³R^{3a})ᵣ-5-6 membered heterocycle substituted with 0-1 R⁵ and consisting of: carbon atoms and 1-4 heteroatoms selected from the group consisting of N, O, and S(O)ₚ;
R^{4a}, at each occurrence, is selected from H, ═O, (CR³R^{3a})ᵣOR², (CR³R^{3a})ᵣ—F, (CR³R^{3a})ᵣ—Br, (CR³R^{3a})ᵣ—Cl, (CR³R^{3a})ᵣI, C₁₋₄ alkyl, (CR³R^{3a})ᵣ—CN, (CR³R^{3a})ᵣNO₂, (CR³R^{3a})ᵣNR²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣN═CHOR³, (CR³R^{3a})ᵣC(O)NH(CH₂)₂NR²R^{2a}, (CR³R^{3a})ᵣNR²C(O)NR²R^{2a}, (CR³R^{3a})ᵣNR²C(O)OR², (CR³R^{3a})ᵣC(═NR²)NR²R^{2a}, (CR³R^{3a})ᵣNHC(═NR²)NR²R^{2a}, (CR³R^{3a})ᵣSO₂NR²R^{2a}, (CR³R^{3a})ᵣNR²SO₂NR²R^{2a}, (CR³R^{3a})ᵣNR²S₂—C₁₋₄ alkyl, (CR³R^{3a})ᵣC(O)NHSO₂—C₁₋₄ alkyl, (CR³R^{3a})ᵣNR²SO₂R⁵, (CR³R^{3a})ᵣS(O)ₚR⁵, (CR³R^{3a})ᵣ(CF₂)ᵣCF₃, (CR³R^{3a})ᵣ-3-10 membered carbocycle substituted with 0-1 R⁵, and a (CR³R^{3a})ᵣ-3-10 membered heterocycle consisting of: carbon atoms and 1-4 heteroatoms selected from the group consisting of N, O, and S(O)ₚ and substituted with 0-1 R⁵;
R^{4b}, at each occurrence, is selected from H, ═O, (CH₂)ᵣOR³, (CH₂)ᵣF, (CH₂)ᵣCl, (CH₂)ᵣBr, (CH₂)ᵣI, C₁₋₄ alkyl, (CH₂)ᵣCN, (CH₂)ᵣNO₂, (CH₂)ᵣNR³R^{3a}, (CH₂)ᵣC(O)R³, (CH₂)ᵣC(O)OR^{3c}, (CH₂)ᵣNR³C(O)R^{3a}, (CH₂)ᵣC(O)NR³R^{3a}, (CH₂)ᵣNR³C(O)NR³R^{3a}, (CH₂)ᵣC(═NR³)NR³R^{3a}, (CH₂)ᵣNR³C(═NR³)NR³R^{3a}, (CH₂)ᵣSO₂NR³R^{3a}, (CH₂)ᵣNR³ SO₂NR³R^{3a}, (CH₂)ᵣNR³SO₂—C₁₋₄ alkyl, (CH₂)ᵣNR³SO₂CF₃,(CH₂)ᵣNR³SO₂-phenyl, (CH₂)ᵣS(O)ₚCF₃, (CH₂)ᵣS(O)ₚ—C₁₋₄ alkyl, (CH₂)ᵣS(O)ₚ-phenyl, and (CH₂)ᵣ(CF₂)ᵣCF₃;
R⁵, at each occurrence, is selected from H, C₁₋₆ alkyl, ═O, (CH₂)ᵣOR³, F, Cl, Br, I, —CN, NO₂, (CH₂)ᵣNR³R^{3a}, (CH₂)ᵣC(O)R³, (CH₂)ᵣC(O)OR^{3c}, (CH₂)ᵣNR³C(O)R^{3a}, (CH₂)ᵣC(O)NR³R^{3a}, (CH₂)ᵣNR³C(O)NR³R^{3a}, (CH₂)ᵣCH(═NOR^{3d}), (CH₂)ᵣC(═NR³)NR³R^{3a}, (CH₂)ᵣNR³C(═NR³)NR³R^{3a}, (CH₂)ᵣSO₂NR³R^{3a}, (CH₂)ᵣNR³SO₂NR³R^{3a}, (CH₂)ᵣNR³SO₂—C₁₋₄ alkyl, (CH₂)ᵣNR³SO₂CF₃, (CH₂)ᵣNR³SO₂-phenyl, (CH₂)ᵣS(O)ₚCF₃, (CH₂)ᵣS(O)ₚ—C₁₋₄ alkyl, (CH₂)ᵣS(O)ₚ-phenyl, (CF₂)ᵣCF₃, phenyl substituted with 0-2 R⁶, naphthyl substituted with 0-2 R⁶, and benzyl substituted with 0-2 R⁶;
R^{5a}, at each occurrence, is selected from C₁₋₆ alkyl, (CH₂)ᵣOR³, (CH₂)ᵣNR³R^{3a}, (CH₂)ᵣC(O)R³, (CH₂)ᵣC(O)OR^{3c}, (CH₂)ᵣNR³C(O)R^{3a}, (CH₂)ᵣC(O)NR³R^{3a}, (CF₂)ᵣCF₃, phenyl substituted with 0-2 R⁶, naphthyl substituted with 0-2 R⁶, and benzyl substituted with 0-2 R⁶, provided that R^{5a} does not form a S—N or S(O)ₚ—C(O) bond;
R⁶, at each occurrence, is selected from H, OH, (CH₂)ᵣOR², halo, C₁₋₄ alkyl, —CN, NO₂, (CH₂)ᵣNR²R^{2a}, (CH₂)ᵣC(O)R^{2b}, NR²C(O)R^{2b}, NR²C(O)NR²R^{2a}, C(═NH)NH₂, NHC(═NH)NH₂, SO₂NR²R^{2a}, NR²SO₂NR²R^{2a}, and NR²SO₂—C₁₋₄ alkyl;
R⁷, at each occurrence, is selected from H, OH, C₁₋₆ alkyl, C₁₋₆ alkyl-C(O)—, C₁₋₆ alkyl-O—, (CH₂)ₙ-phenyl, C₁₋₆ alkyl-OC(O)—, C₆₋₁₀ aryl-O—, C₆₋₁₀ aryl-OC(O)—, C₆₋₁₀ aryl-CH₂—C(O)—, C₁₋₄ alkyl-C(O)O—C₁₋₄ alkyl-OC(O)—, C₆₋₁₀ aryl-C(O)O—C₁₋₄ alkyl-OC(O)—, C₁₋₆ alkyl-NH₂—C(O)—, phenyl-NH₂—C(O)—, and phenyl C₀₋₄ alkyl-C(O)—;
R⁸, at each occurrence, is selected from H, C₁₋₆ alkyl, and (CH₂)ₙ-phenyl;
alternatively, NR⁷R⁸ forms a 5-10 membered heterocyclic ring consisting of carbon atoms and 0-2 additional heteroatoms selected from the group consisting of N, O, and S(O)ₚ;
R⁹, at each occurrence, is selected from H, C₁₋₆ alkyl, and (CH₂)ₙ-phenyl;
n, at each occurrence, is selected from 0, 1, 2, and 3;
p, at each occurrence, is selected from 0, 1, and 2;
r, at each occurrence, is selected from 0, 1, 2, 3, 4, 5, and 6; and
t, at each occurrence, is selected from 0, 1, 2, and 3.

2. A compound according to claim 1, wherein:
rings M and N are substituted with 0-2 R^{1a} and 0-2 R³;
one of P₁ and M₁ is -Z-A-B and the other -G₁-G;
G is a group of formula IIa or IIb: 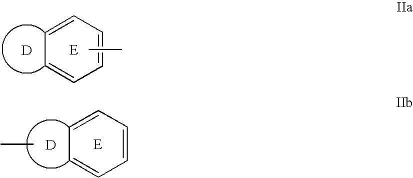
ring D, including the two atoms of ring E to which it is attached, is a 5-6 membered ring consisting of: carbon atoms and 0-2 heteroatoms selected from the group consisting of N, O, and S(O)ₚ;
ring D is substituted with 0-2 R and there are 0-3 ring double bonds;
E is selected from phenyl, pyridyl, pyrimidyl, pyrazinyl, and pyridazinyl, and is substituted with 1-2 R;
alternatively, ring D is absent, and ring E is selected from phenyl, pyridyl, pyrimidyl, and thienyl, and ring E is substituted with 1-2 R;
alternatively, ring D is absent, ring E is selected from phenyl, pyridyl, and thienyl, and ring E is substituted with either phenyl or a 5-6 membered heterocycle consisting of: carbon atoms and 1-4 heteroatoms selected from the group consisting of N, O, and S(O)ₚ, wherein the phenyl is substituted with 1-2 R and the 5-6 membered heterocycle is substituted with 0-1 carbonyls and 1-2 R and has 0-3 ring double bonds;
R is selected from H, C₁₋₄ alkyl, F, Cl, OH, OCH₃, OCH₂CH₃, OCH(CH₃)₂, CN, C(═NH)NH₂, NH₂, NH(C₁₋₃ alkyl), N(C₁₋₃ alkyl)₂, C(═NH)NH₂, CH₂NH₂, CH₂NH(C₁₋₃ alkyl), CH₂N(C₁₋₃ alkyl)₂, (CR⁸R⁹)ₜNR⁷R⁸, C(O)NR⁷R⁸, CH₂C(O)NR⁷R⁸, NHC(O)R⁷, S(O)ₚNR⁷R⁸, CH₂S(O)ₚNR⁷R⁸, and OCF₃;
alternatively, when 2 R groups are attached to adjacent atoms, they combine to form methylenedioxy or ethylenedioxy;
A is selected from:
C₅₋₁₀ carbocycle substituted with 0-2 R⁴, and
5-10 membered heterocycle substituted with 0-2 R⁴ and consisting of: carbon atoms and 1-4 heteroatoms selected from the group consisting of N, O, and S(O)ₚ;
B is selected from Y, X—Y, CH₂NR²R^{2a}, and CH₂CH₂NR²R^{2a};
X is selected from —CR²R^{2a})₁₋₄—, —C(O)—, —C(O)CR²R^{2a}—, —CR²R^{2a}C(O), —S(O)₂—, —S(O)₂CR²R^{2a}—, —CR²R^{2a}S(O)₂—, —NR²S(O)₂—, —NR²S(O)₂—, —NR²C(O)—, —C(O)NR²—, NR², NR²CR²R^{2a}—, —CR²R^{2a}NR²—, O, —OCR²R^{2a}—, and —CR²R^{2a}O—;
Y is selected from one of the following rings and is substituted with 0-2 R^{4a};
cyclopropyl, cyclopentyl, cyclohexyl, phenyl, piperidinyl, piperazinyl, pyridyl, pyrimidyl, furanyl, morpholinyl, thiophenyl, pyrrolyl, pyrrolidinyl, oxazolyl, isoxazolyl, isoxazolinyl, thiazolyl, isothiazolyl, pyrazolyl, imidazolyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, 1,2,3-triazolyl, 1,2,4-triazolyl, 1,2,5-triazolyl, 1,3,4-triazolyl, benzofuranyl, benzothiofuranyl, indolyl, benzimidazolyl, benzoxazolyl, benzthiazolyl, indazolyl, benzisoxazolyl, benzisothiazolyl, and isoindazolyl;
alternatively, Y is selected from the following bicyclic heteroaryl ring systems: 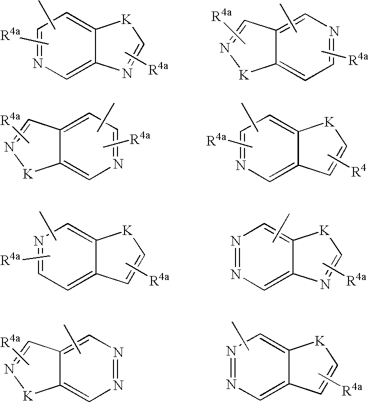
K is selected from O, S, NH, and N;
G₁ is selected from ═O or a 2-5 membered linear chain consisting of: 0-5 carbon atoms, 0-2 carbonyl groups, and 0-3 heteroatoms selected from O, N, and S(O)ₚ, and G₁ is substituted with 0-4 R³, and there are 0-1 double bonds; provided that other than an S—S, S—O, or O—O bond is present in G₁;
Z is selected from —(CR³R^{3e})₂₋₃—, (CR³R^{3e})_{q}C(O)(CR³R^{3e})_{q1}, (CR³R^{3e})_{q}O(CR³R^{3e})_{q1}, (CR³R^{3e})_{q}NR^{3b}(CR³R^{3e})_{q1}, (CR³R^{3e})_{q}C(O)NR^{3b}(CR³R^{3e})_{q1}, (CR³R^{3e})_{q}NR^{3b}C(O)(CR³R^{3e})_{q1}, (CR³R^{3e})_{q}NR^{3b}C(O)NR^{3b}(CR³R^{3e})_{q1}, (CR³R^{3e})_{q}NR^{3b}(CR³R^{3e})_{q}C(O)NR^{3b}(CR³R^{3e})_{q1}, (CR³R^{3e})_{q}S(CR³R^{3e})_{q1}, (CR³R^{3e})_{q}S(O)(CR³R^{3e})_{q1}, (CR³R^{3e})_{q}S(O)₂(CR³R^{3e})_{q1}, (CR³R^{3e})_{q}SO₂NR^{3b}(CR³R^{3e})_{q1}, (CR³R^{3e})_{q}NR^{3b}SO₂(CR³R^{3e})_{q1}, and (CR³R^{3e})_{q}C(O)NR^{3b}S(O)₂(CR³R^{3e})_{q1}, wherein q+q1 total 1 or 2, provided that Z does not form a N—S, NCH₂N, NCH₂O, or NCH₂S bond with either group to which it is attached;
R^{1a}, at each occurrence, is selected from H, —(CH₂)ᵣR^{1b}, —(CH(CH₃))ᵣR^{1b}, —(C(CH₃)₂)ᵣR^{1b}, —O—(CR³R^{3a})ᵣR^{1b}, —NR²—(CR³R^{3a})ᵣR^{1b}, and —S—(CR³R^{3a})ᵣR^{1b}, provided that R^{1a} forms other than an N-halo, N—S, O—O, or N—CN bond;
provided that R^{1a} is other than a substituted or unsubstituted 3,4-dihydroxyphenyl group;
alternatively, when two R^{1a} groups are attached to adjacent atoms or to the same carbon atom, together with the atoms to which they are attached they form a 5-7 membered ring consisting of: carbon atoms and 0-2 heteroatoms selected from the group consisting of N, O, and S(O)ₚ, this ring being substituted with 0-2 R^{4b} and 0-3 ring double bonds;
R^{1b} is selected from H, CH₃, CH₂CH₃, CH₂CH₂CH₃, CH(CH₃)₂, F, Cl, Br, I, —CN, —CF₃, OR², NR²R^{2a}, C(O)R^{2b}, CO₂R^{2b}, OC(O)R², CO₂R^{2a}, S(O)ₚR², NR²(CH₂)ᵣOR², NR²C(O)R^{2b}, NR²C(O)NHR², NR²C(O)₂R^{2a}, OC(O)NR²R^{2a}, C(O)NR²R^{2a}, C(O)NR²(CH₂)ᵣOR², SO₂NR²R^{2a}, NR²SO₂R², C₅₋₆ carbocycle substituted with 0-2 R^{4b}, and 5-6 membered heterocycle consisting of carbon atoms and from 1-4 heteroatoms selected from the group consisting of N, O, and S(O)ₚ and substituted with 0-2 R^{4b}, provided that R^{1b} forms other than an O—O, N-halo, N—S, or N—CN bond;
R², at each occurrence, is selected from H, CF₃, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, C₅₋₆ carbocycle substituted with 0-2 R^{4b}, a C₅₋₆ carbocycle-CH₂-substituted with 0-2 R^{4b}, and 5-6 membered heterocycle substituted with 0-2 R^{4b} and consisting of: carbon atoms and 1-4 heteroatoms selected from the group consisting of N, O, and S(O)ₚ;
R^{2a}, at each occurrence, is selected from H, CF₃, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, C₅₋₆ carbocycle substituted with 0-2 R^{4b}, and 5-6 membered heterocycle substituted with 0-2 R^{4b} and consisting of: carbon atoms and 1-4 heteroatoms selected from the group consisting of N, O, and S(O)ₚ;
alternatively, NR²R^{2a} forms a 5 or 6 membered saturated, partially saturated or unsaturated ring substituted with 0-2 R^{4b} and consisting of: 0-1 additional heteroatoms selected from the group consisting of N, O, and S(O)ₚ;
R^{2b}, at each occurrence, is selected from CF₃, C₁₋₄ alkoxy, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, C₅₋₆ carbocycle substituted with 0-2 R^{4b}, and 5-6 membered heterocycle substituted with 0-2 R^{4b} and consisting of: carbon atoms and 1-4 heteroatoms selected from the group consisting of N, O, and S(O)ₚ;
R^{2c}, at each occurrence, is selected from CF₃, OH, C₁₋₄ alkoxy, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, benzyl, C₅₋₆ carbocycle substituted with 0-2 R^{4b}, and 5-6 membered heterocycle substituted with 0-2 R^{4b} and consisting of carbon atoms and from 1-4 heteroatoms selected from the group consisting of N, O, and S(O)ₚ;
R³, at each occurrence, is selected from H, CH₃, CH₂CH₃, CH₂CH₂CH₃, CH(CH₃)₂, benzyl, and phenyl;
R^{3a}, at each occurrence, is selected from H, CH₃, CH₂CH₃, CH₂CH₂CH₃, CH(CH₃)₂, benzyl, and phenyl;
alternatively, NR³R^{3a} forms a 5 or 6 membered saturated, partially unsaturated, or unsaturated ring consisting of: carbon atoms and the nitrogen atom to which R³ and R^{3a} are attached;
R^{3c}, at each occurrence, is selected from CH₃, CH₂CH₃, CH₂CH₂CH₃, CH(CH₃)₂, benzyl, and phenyl;
R^{3d}, at each occurrence, is selected from H, CH₃, CH₂CH₃, CH₂CH₂CH₃, CH(CH₃)₂, CH₂-phenyl, CH₂CH₂-phenyl, and C(═O)R^{3c};
R⁴, at each occurrence, is selected from H, ═O, OR², CH₂OR², (CH₂)₂OR², F, Cl, Br, I, C₁₋₄ alkyl, —CN, NO₂, NR²R^{2a}, CH₂NR²R^{2a}, (CH₂)₂NR²R^{2a}, C(O)R^{2c}, NR²C(O)R^{2b}, C(O)NR²R^{2a}, SO₂NR²R^{2a}, S(O)ₚR^{5a}, CF₃, CF₂CF₃, 5-6 membered carbocycle substituted with 0-1 R⁵, and a 5-6 membered heterocycle substituted with 0-1 R⁵ and consisting of: carbon atoms and 1-4 heteroatoms selected from the group consisting of N, O, and S(O)ₚ;
R^{4a}, at each occurrence, is selected from H, ═O, (CR³R^{3a})ᵣOR², (CR³R^{3a})ᵣ—F, (CR³R^{3a})ᵣ—Br, (CR³R^{3a})ᵣCl, C₁₋₄ alkyl, (CR³R^{3a})ᵣ—CN, (CR³R^{3a})ᵣNO₂, (CR³R^{3a})ᵣNR²R^{2a}, (CR³R^{3a})ᵣC(O)R^{2c}, (CR³R^{3a})ᵣNR²C(O)R^{2b}, (CR³R^{3a})ᵣC(O)NR²R^{2a}, (CR³R^{3a})ᵣSO₂NR²R^{2a}, (CR³R^{3a})ᵣNR²SO₂NR²R^{2a}, (CR³R^{3a})ᵣNR²SO₂—C₁₋₄ alkyl, (CR³R^{3a})ᵣC(O)NHSO₂—C₁₋₄ alkyl, (CR³R^{3a})ᵣNR²SO₂R⁵, (CR³R^{3a})ᵣS(O)ₚR⁵, (CR³R^{3a})ᵣ(CF₂)ᵣCF₃, phenyl substituted with 0-1 R⁵, and a 5 membered aromatic heterocycle consisting of: carbon atoms and 1-3 heteroatoms selected from the group consisting of N, O, and S(O)ₚ substituted with 0-1 R⁵;
R^{4b}, at each occurrence, is selected from H, ═O, OR³, CH₂OR³, F, Cl, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —CN, NO₂, NR³R^{3a}, CH₂NR³R^{3a}, C(O)R³, CH₂—C(O)R³, C(O)OR^{3c}, CH₂C(O)OR^{3c}, NR³C(O)R^{3a}, CH₂NR³C(O)R^{3a}, C(O)NR³R^{3a}, CH₂C(O)NR³R^{3a}, NR³C(O)NR³R^{3a}, CH₂NR³C(O)NR³R^{3a}, C(═NR³)NR³R^{3a}, CH₂C(═NR³)NR³R^{3a}, NR³C(═NR³)NR³R^{3a}, CH₂NR³C(═NR³)NR³R^{3a}, SO₂NR³R^{3a}, CH₂SO₂NR³R^{3a}, NR³ SO₂NR³R^{3a}, CH₂NR³SO₂NR³R^{3a}, NR³SO₂—C₁₋₄ alkyl, CH₂NR³SO₂—C₁₋₄ alkyl, NR³SO₂CF₃, CH₂NR³ SO₂CF₃, NR³SO₂-phenyl, CH₂NR³SO₂-phenyl, S(O)ₚCF₃, CH₂S(O)ₚCF₃, S(O)ₚ—C₁₋₄ alkyl, CH₂S(O)ₚ—C₁₋₄ alkyl, S(O)ₚ-phenyl, CH₂S(O)ₚ-phenyl, CF₃, and CH₂—CF₃;
R⁵, at each occurrence, is selected from H, ═O, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, OR³, CH₂OR³, F, Cl, —CN, NO₂, NR³R^{3a}, CH₂NR³R^{3a}, C(O)R³, CH₂C(O)R³, C(O)OR^{3c}, CH₂C(O)OR^{3c}, NR³C(O)R^{3a}, C(O)NR³R^{3a}, NR³C(O)NR³R^{3a}, CH(═NOR^{3d}), C(═NR³)NR³R^{3a}, NR³C(═NR³)NR³R^{3a}, SO₂NR³R^{3a}, NR³SO₂NR³R^{3a}, NR³SO₂—C₁₋₄ alkyl, NR³SO₂CF₃, NR³SO₂-phenyl, S(O)ₚCF₃, S(O)ₚ—C₁₋₄ alkyl, S(O)ₚ-phenyl, CF₃, phenyl substituted with 0-2 R⁶, naphthyl substituted with 0-2 R⁶, and benzyl substituted with 0-2 R⁶;
R⁶, at each occurrence, is selected from H, OH, OR², F, Cl, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —CN, NO₂, NR²R^{2a}, CH₂NR²R^{2a}, C(O)R^{2b}, CH₂C(O)R^{2b}, NR²C(O)R^{2b}, NR²C(O)NR²R^{2a}, C(═NH)NH₂, NHC(═NH)NH₂, SO₂NR²R^{2a}, NR²SO₂NR²R^{2a}, and NR²SO₂C₁₋₄ alkyl; and
r, at each occurrence, is selected from 0, 1, 2, and 3.

3. A compound according to claim 2, wherein:
rings M and N are substituted with 0-1 R^{1a} and 0-2 R³;
G is selected from the group: 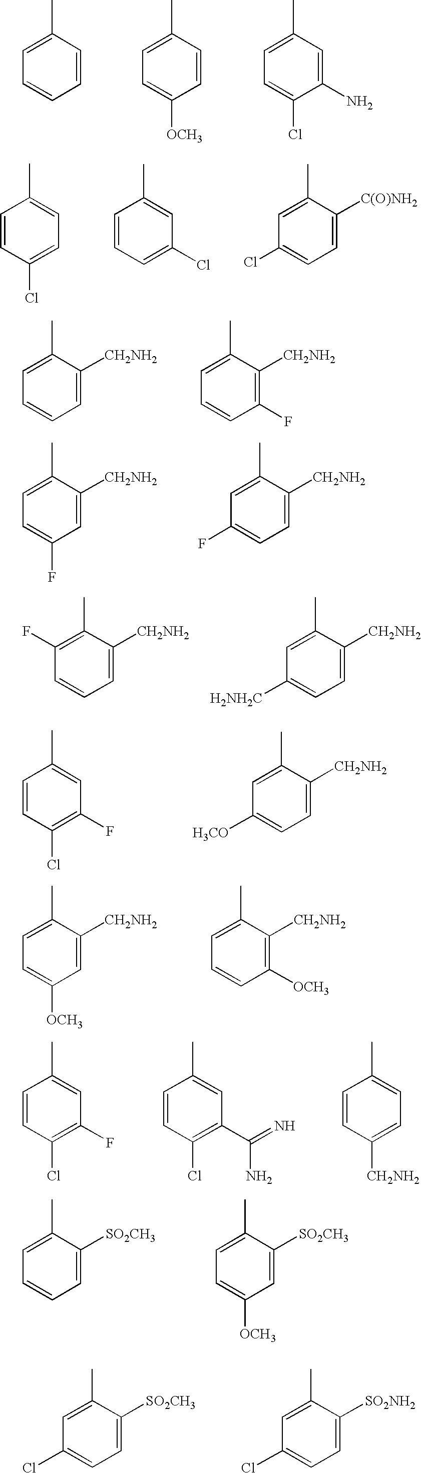 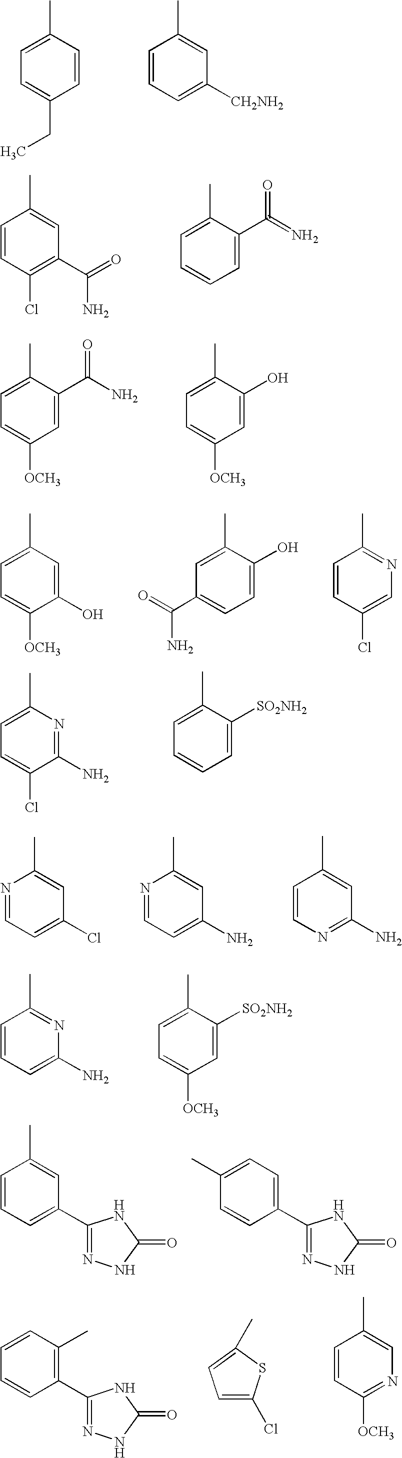 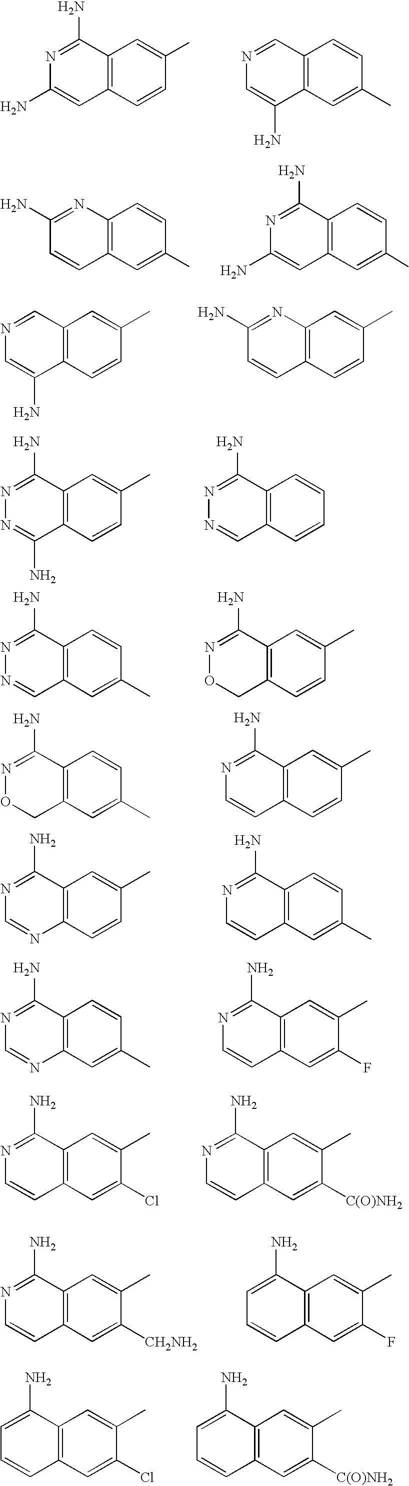 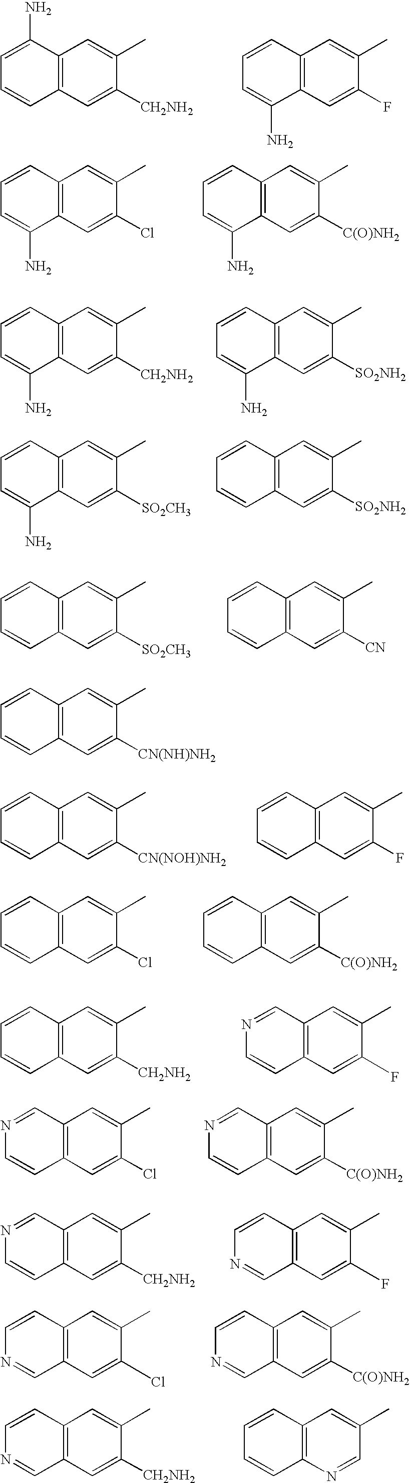 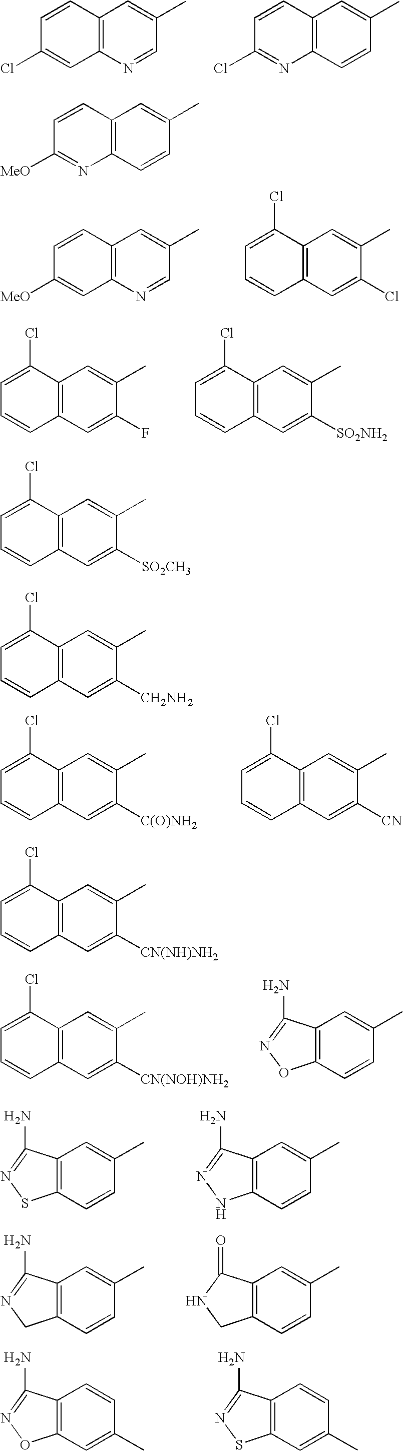 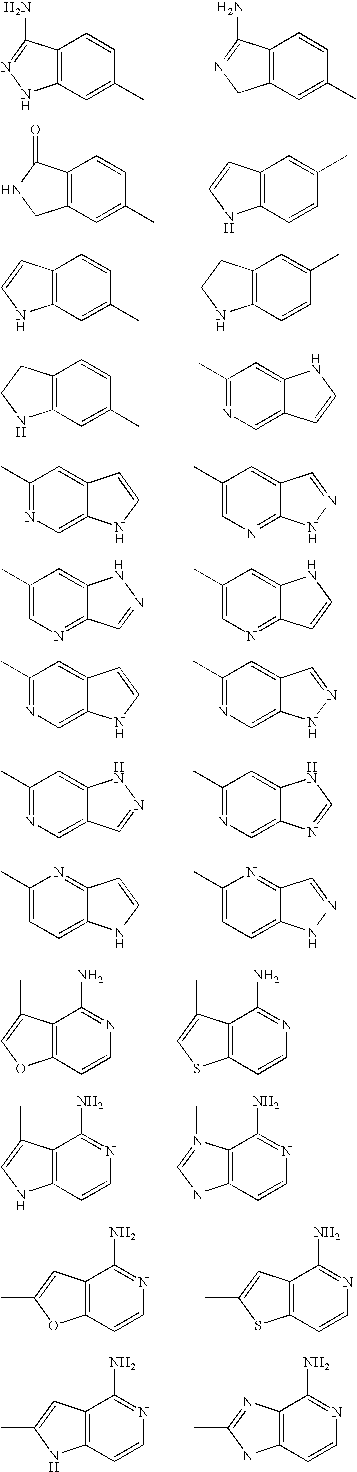 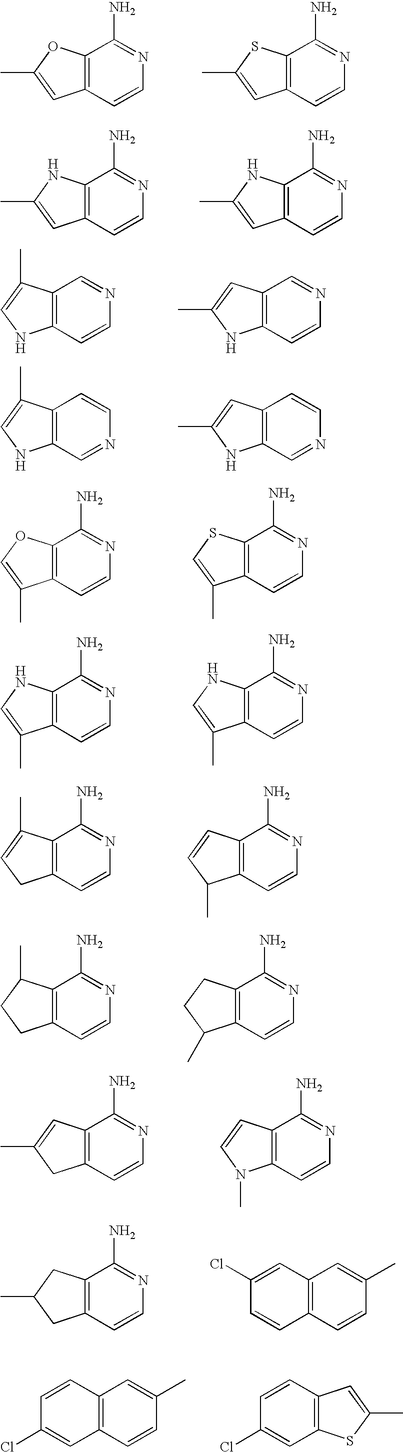 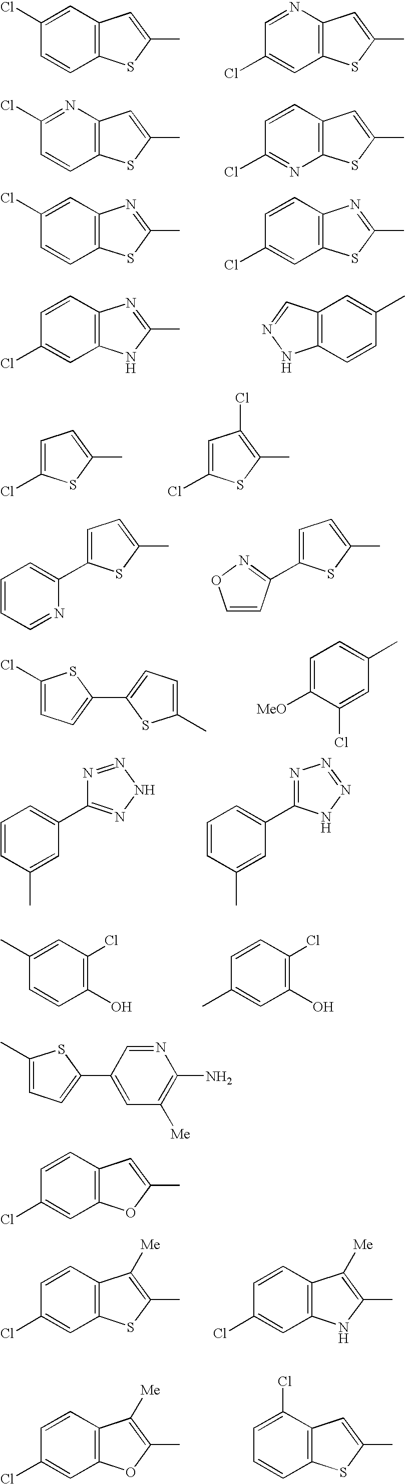 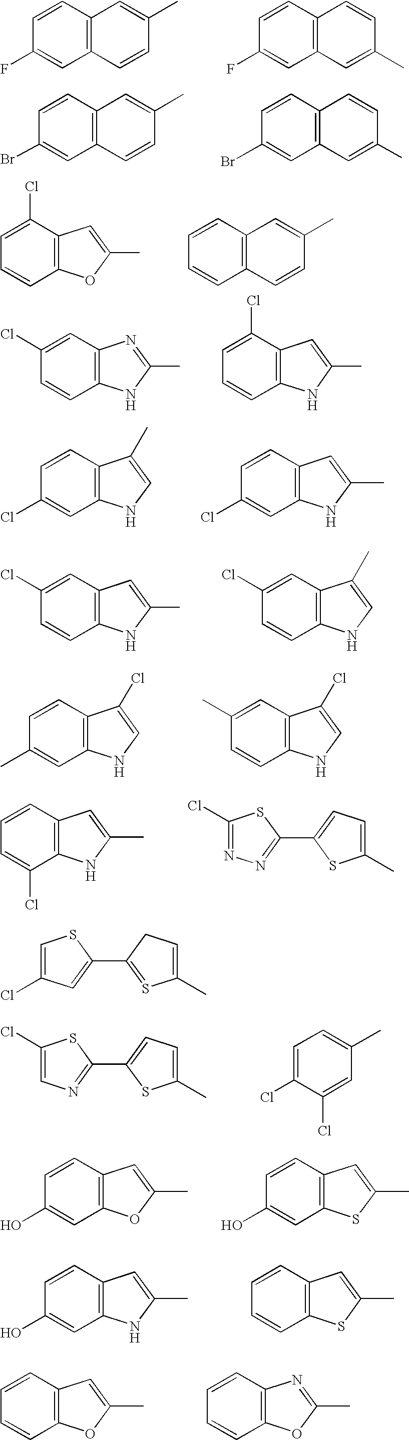 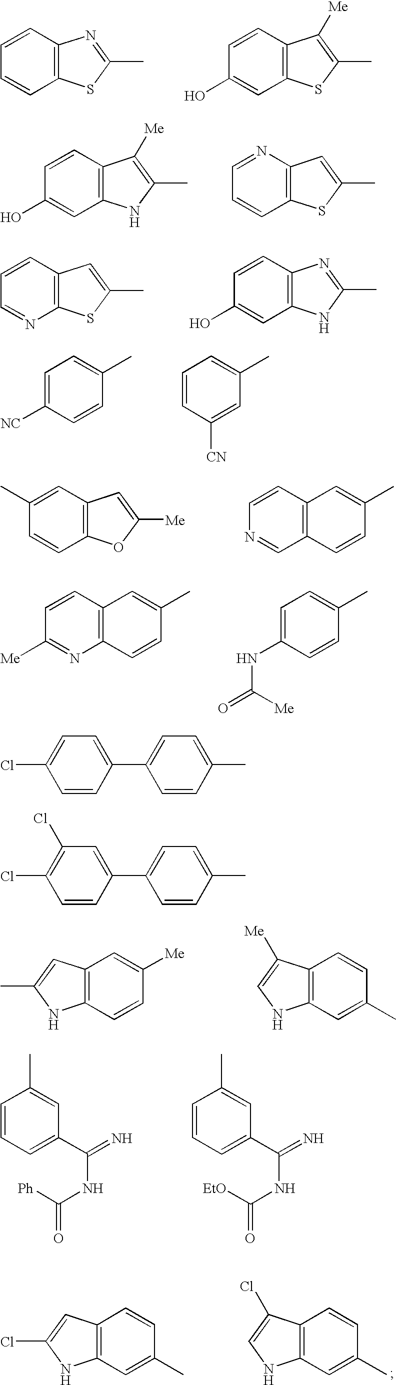
G₁ is selected from ═O or a 2-4 membered linear chain consisting of: 0-4 carbon atoms, 0-1 carbonyl groups, and 0-2 heteroatoms selected from O, N, and S(O)ₚ, and G₁ is substituted with 0-4 R³, and there are 0-1 double bonds; provided that other than an S—S, S—O, or O—O bond is present in G₁;
Z is selected from (CR³R^{3e})_{q}C(O)(CR³R^{3e})_{q1}, (CR³R^{3e})_{q}NR^{3b}(CR³R^{3e})_{q1}, (CR³R^{3e})_{q}C(O)NR^{3b}(CR³R^{3e})_{q1}, (CR³R^{3e})_{q}NR^{3b}C(O)(CR³R^{3e})_{q1}, (CR³R^{3e})_{q}NR^{3b}C(O)NR^{3b}(CR³R^{3e})_{q1}, (CR³R^{3e})_{q}S(O)₂(CR³R^{3e})_{q1}, (CR³R^{3e})_{q}SO₂NR^{3b}(CR³R^{3e})_{q1}, (CR³R^{3e})_{q}NR^{3b}SO₂(CR³R^{3e})_{q1}, wherein q+q1 total 1 or 2, provided that Z does not form a N—S, NCH₂N, NCH₂O, or NCH₂S bond with either group to which it is attached;
A is selected from one of the following carbocycles and heterocycles which are substituted with 0-2 R⁴;
cyclohexyl, phenyl, piperidinyl, piperazinyl, pyridyl, pyrimidyl, furanyl, morpholinyl, thienyl, pyrrolyl, pyrrolidinyl, oxazolyl, isoxazolyl, thiazolyl, isothiazolyl, pyrazolyl, imidazolyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, 1,2,3-triazolyl, 1,2,4-triazolyl, 1,2,5-triazolyl, 1,3,4-triazolyl, benzofuranyl, benzothiofuranyl, indolinyl, indolyl, benzimidazolyl, benzoxazolyl, benzthiazolyl, indazolyl, benzisoxazolyl, benzisothiazolyl, and isoindazolyl;
X is selected from —(CR²R^{2a})₁₋₂—, —C(O)—, —S(O)₂—, —NR²S(O)₂—, —NR²S(O)₂—, —NR²C(O)—, —C(O)NR²—, NR², —NR²CR²R^{2a}—, —CR²R^{2a}NR²—, O, OCR²R^{2a}—, and —CR²R^{2a}O—;
R^{1a}, at each occurrence, is selected from H, R^{1b}, CH(CH₃)R^{1b}, C(CH₃)₂R^{1b}, CH₂R^{1b}, and CH₂CH₂R^{1b}, provided that R^{1a} forms other than an N-halo, N—S, or N—CN bond;
provided that R^{1a} is other than a substituted or unsubstituted 3,4-dihydroxyphenyl group;
alternatively, when two R^{1a} groups are attached to adjacent atoms or to the same carbon atom, together with the atoms to which they are attached, they form a 5-6 membered ring consisting of: carbon atoms and 0-2 heteroatoms selected from the group consisting of N, O, and S(O)ₚ, this ring being substituted with 0-2 R^{4b} and comprising: 0-3 ring double bonds;
R^{1b} is selected from H, CH₃, CH₂CH₃, F, Cl, Br, —CN, CF₃, OR², NR²R^{2a}, C(O)R^{2b}, CO₂R^{2b}, OC(O)R², CO₂R^{2a}, S(O)ₚR², NR²(CH₂)ᵣOR², NR²C(O)R^{2b}, C(O)NR²R^{2a}, SO₂NR²R^{2a}, NR²SO₂R², phenyl substituted with 0-2 R^{4b}, and 5-6 membered aromatic heterocycle consisting of carbon atoms and from 1-4 heteroatoms selected from the group consisting of N, O, and S(O)ₚ and substituted with 0-2 R^{4b}, provided that R^{1b} forms other than an O—O, N-halo, N—S, or N—CN bond;
R², at each occurrence, is selected from H, CF₃, CH₃, CH₂CH₃, CH₂CH₂CH₃, CH(CH₃)₂, phenyl substituted with 0-2 R^{4b}, a benzyl substituted with 0-2 R^{4b}, and 5-6 membered aromatic heterocycle substituted with 0-2 R^{4b} and consisting of: carbon atoms and 1-4 heteroatoms selected from the group consisting of N, O, and S(O)ₚ;
R^{2a}, at each occurrence, is selected from H, CF₃, CH₃, CH₂CH₃, CH₂CH₂CH₃, CH(CH₃)₂, benzyl, phenyl substituted with 0-2 R^{4b}, and 5-6 membered aromatic heterocycle substituted with 0-2 R^{4b} and consisting of: carbon atoms and 1-4 heteroatoms selected from the group consisting of N, O, and S(O)ₚ;
R^{2b}, at each occurrence, is selected from CF₃, C₁₋₄ alkoxy, CH₃, CH₂CH₃, CH₂CH₂CH₃, CH(CH₃)₂, benzyl, phenyl substituted with 0-2 R^{4b}, and 5-6 membered aromatic heterocycle substituted with 0-2 R^{4b} and consisting of: carbon atoms and 1-4 heteroatoms selected from the group consisting of N, O, and S(O)ₚ;
R^{2c}, at each occurrence, is selected from CF₃, OH, OCH₃, OCH₂CH₃, OCH₂CH₂CH₃, OCH(CH₃)₂, CH₃, CH₂CH₃, CH₂CH₂CH₃, CH(CH₃)₂, benzyl, phenyl substituted with 0-2 R^{4b}, and 5-6 membered aromatic heterocycle substituted with 0-2 R^{4b} and consisting of carbon atoms and from 1-4 heteroatoms selected from the group consisting of N, O, and S(O)ₚ;
alternatively, NR²R^{2a} forms a 5 or 6 membered saturated, partially saturated or unsaturated ring substituted with 0-2 R^{4b} and consisting of: 0-1 additional heteroatoms selected from the group consisting of N, O, and S(O)ₚ;
R⁴, at each occurrence, is selected from H, (CH₂)₂OR², CH₂OR², OR², F, Cl, Br, I, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, —CN, NO₂, NR²R^{2a}, CH₂NR²R^{2a}, (CH₂)₂NR²R^{2a}, C(O)R^{2c}, NR²C(O)R^{2b}, C(O)NR²R^{2a}, SO₂NR²R^{2a}, CF₃, and CF₂CF₃;
R^{4a}, at each occurrence, is selected from H, ═O, (CH₂)ᵣOR², (CH₂)ᵣ—F, (CH₂)ᵣ—Br, (CH₂)ᵣC₁, C₁₋₄ alkyl, (CH₂)ᵣCN, (CH₂)ᵣNO₂, (CH₂)ᵣNR²R^{2a}, (CH₂)ᵣC(O)R^{2c}, (CH₂)ᵣNR²C(O)R^{2b}, (CH₂)ᵣC(O)NR²R^{2a}, (CH₂)ᵣSO₂NR²R^{2a}, (CH₂)ᵣNR²SO₂NR²R^{2a}, (CH₂)ᵣNR²SO₂—C₁₋₄ alkyl, (CH₂)ᵣC(O)NHSO₂—C₁₋₄ alkyl, (CH₂)ᵣNR²SO₂R⁵, (CH₂)ᵣS(O)ₚR⁵, (CH₂)ᵣ(CF₂)ᵣCF₃, phenyl substituted with 0-1 R⁵, and a 5 membered aromatic heterocycle consisting of: carbon atoms and 1-3 heteroatoms selected from the group consisting of N, O, and S(O)ₚ substituted with 0-1 R⁵;
R^{4b}, at each occurrence, is selected from H, ═O, OR³, CH₂OR³, F, Cl, CH₃, CH₂CH₃, CH₂CH₂CH₃, CH(CH₃)₂, —CN, NO₂, NR³R^{3a}, CH₂NR³R^{3a}, C(O)R³, CH₂—C(O)R³, C(O)OR^{3c}, CH₂—C(O)OR^{3c}, NR³C(O)R^{3a}, CH₂NR³C(O)R^{3a}, C(O)NR³R^{3a}, CH₂—C(O)NR³R^{3a}, SO₂NR³R^{3a}, CH₂SO₂NR³R^{3a}, NR³SO₂—C₁₋₄ alkyl, CH₂NR³SO₂—C₁₋₄ alkyl, NR³SO₂-phenyl, CH₂NR³SO₂-phenyl, S(O)ₚCF₃, CH₂S(O)ₚCF₃, S(O)ₚ—C₁₋₄ alkyl, CH₂S(O)ₚ—C₁₋₄ alkyl, S(O)ₚ-phenyl, CH₂S(O)ₚ-phenyl, and CF₃;
R⁵, at each occurrence, is selected from H, ═O, CH₃, CH₂CH₃, CH₂CH₂CH₃, CH(CH₃)₂, OR³, CH₂OR³, F, Cl, —CN, NO₂, NR³R^{3a}, CH₂NR³R^{3a}, C(O)R³, CH₂C(O)R³, C(O)OR^{3c}, CH₂C(O)OR^{3c}, NR³C(O)R^{3a}, C(O)NR³R^{3a}, SO₂NR³R^{3a}, NR³SO₂—C₁₋₄ alkyl, NR³SO₂CF₃, NR³SO₂-phenyl, S(O)ₚCF₃, S(O)ₚ—C₁₋₄ alkyl, S(O)ₚ-phenyl, CF₃, phenyl substituted with 0-2 R⁶, naphthyl substituted with 0-2 R⁶, and benzyl substituted with 0-2 R⁶;
R⁶, at each occurrence, is selected from H, OH, OR², F, Cl, CH₃, CH₂CH₃, CH₂CH₂CH₃, CH(CH₃)₂, —CN, NO₂, NR²R^{2a}, CH₂NR²R^{2a}, C(O)R^{2b}, CH₂C(O)R^{2b}, NR²C(O)R^{2b}, SO₂NR²R^{2a}, and NR²SO₂C₁₋₄ alkyl; and
r, at each occurrence, is selected from 0, 1, and 2.

4. A compound according to claim 3, wherein:
G is selected from the group: 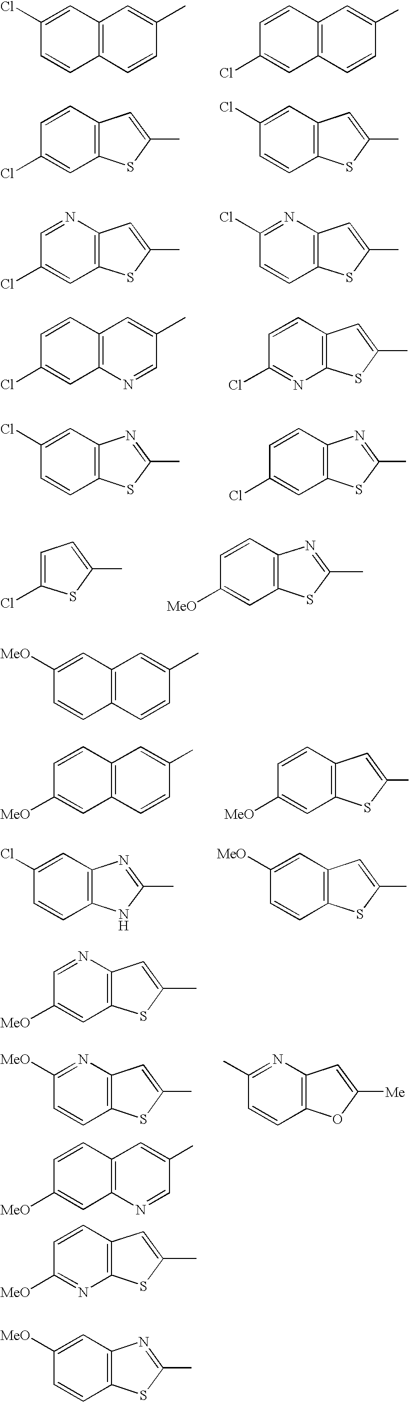 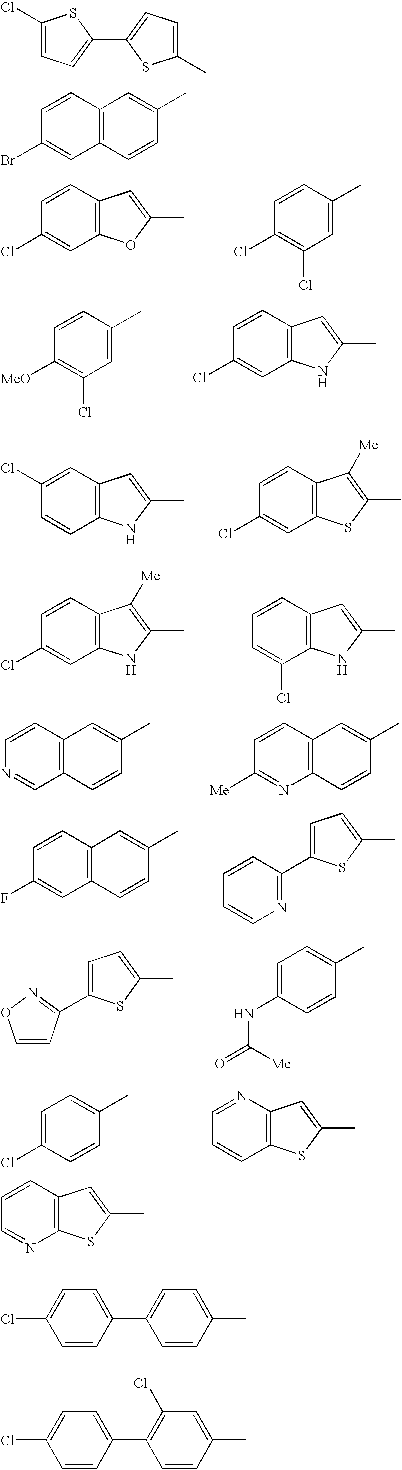 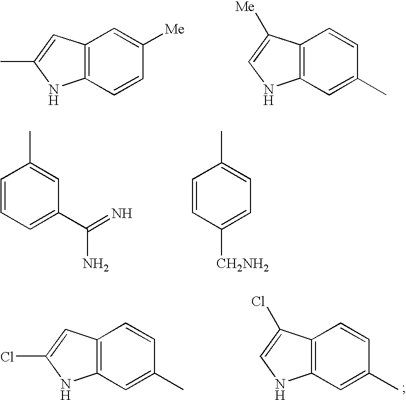
A is selected from cyclohexyl, indolinyl, piperidinyl, phenyl, pyridyl, and pyrimidyl, and is substituted with 0-2 R⁴;
X is selected from CH₂, C(O), —S(O)₂—, —NHC(O)—, —C(O)NH—, —CH₂NH—, O, and —CH₂O—;
Z is selected from C(O), CH₂NH, NHCH₂, C(O)NH, NHC(O), NHC(O)NH, S(O)₂, SO₂NH, and NHSO₂, wherein the left side of Z is attached to ring M, provided that Z does not form a N—S, NCH₂N, NCH₂O, or NCH₂S bond with either group to which it is attached;
R^{1a}, at each occurrence, is selected from H, R^{1b}, CH(CH₃)R^{1b}, C(CH₃)₂R^{1b}, and CH₂R^{1b}, provided that R^{1a} forms other than an N-halo, N—S, or N—CN bond;
R^{1b} is selected from CH₃, CH₂CH₃, F, Cl, Br, —CN, CF₃, OR², NR²R^{2a}, C(O)R^{2b}, CO₂R^{2b}, CO₂R^{2a}, S(O)ₚR², C(O)NR²R^{2a}, SO₂NR²R^{2a}, NR²SO₂R², and 5-6 membered aromatic heterocycle consisting of carbon atoms and from 1-4 heteroatoms selected from the group consisting of N, O, and S(O)ₚ and substituted with 0-2 R^{4b}, provided that R^{1b} forms other than an O—O, N-halo, N—S, or N—CN bond;
R², at each occurrence, is selected from H, CH₃, CH₂CH₃, CH₂CH₂CH₃, CH(CH₃)₂, phenyl substituted with 0-1 R^{4b}, benzyl substituted with 0-1 R^{4b}, and 5-6 membered aromatic heterocycle substituted with 0-1 R^{4b} and consisting of: carbon atoms and 1-4 heteroatoms selected from the group consisting of N, O, and S(O)ₚ;
R^{2a}, at each occurrence, is selected from H, CH₃, CH₂CH₃, CH₂CH₂CH₃, CH(CH₃)₂, benzyl, phenyl substituted with 0-1 R^{4b}, and 5-6 membered aromatic heterocycle substituted with 0-1 R^{4b} and consisting of: carbon atoms and 1-4 heteroatoms selected from the group consisting of N, O, and S(O)ₚ;
alternatively, NR²R^{2a} forms a 5 or 6 membered saturated, partially saturated or unsaturated ring substituted with 0-1 R^{4b} and consisting of: 0-1 additional heteroatoms selected from the group consisting of N, O, and S(O)ₚ;
R^{2b}, at each occurrence, is selected from OH, OCH₃, OCH₂CH₃, OCH₂CH₂CH₃, OCH(CH₃)₂, CH₃, CH₂CH₃, CH₂CH₂CH₃, CH(CH₃)₂, benzyl, phenyl substituted with 0-1 R^{4b}, and 5-6 membered aromatic heterocycle substituted with 0-1 R^{4b} and consisting of: carbon atoms and 1-4 heteroatoms selected from the group consisting of N, O, and S(O)ₚ;
R^{2c}, at each occurrence, is selected from OH, OCH₃, OCH₂CH₃, OCH₂CH₂CH₃, OCH(CH₃)₂, CH₃, CH₂CH₃, CH₂CH₂CH₃, CH(CH₃)₂, benzyl, phenyl substituted with 0-1 R^{4b}, and 5-6 membered aromatic heterocycle substituted with 0-1 R^{4b} and consisting of carbon atoms and from 1-4 heteroatoms selected from the group consisting of N, O, and S(O)ₚ;
R⁴, at each occurrence, is selected from OH, OR², CH₂OR², (CH₂)₂OR², F, Br, Cl, I, CH₃, CH₂CH₃, CH₂CH₂CH₃, CH(CH₃)₂, CH₂CH₂CH₂CH₃, CH₂CH(CH₃)₂, CH(CH₃)CH₂CH₃, C(CH₃)₃, NR²R^{2a}, CH₂NR²R^{2a}, (CH₂)₂NR²R^{2a}, CF₃, and CF₂CF₃;
R^{4a}, at each occurrence, is selected from H, ═O, (CH₂)ᵣOR², F, Br, C₁, C₁₋₄ alkyl, (CH₂)ᵣNR²R^{2a}, (CH₂)ᵣC(O)R^{2c}, (CH₂)ᵣNR²C(O)R^{2b}, (CH₂)ᵣC(O)NR²R^{2a}, (CH₂)ᵣSO₂NR²R^{2a}, (CH₂)ᵣNR^{2s}O₂R⁵, (CH₂)ᵣS(O)ₚR⁵, (CH₂)ᵣ(CF₂)ᵣCF₃, phenyl substituted with 0-1 R⁵, and a 5 membered aromatic heterocycle consisting of: carbon atoms and 1-3 N and is substituted with 1 R⁵;
R^{4b}, at each occurrence, is selected from H, ═O, OR³, CH₂OR³, F, Cl, CH₃, CH₂CH₃, CH₂CH₂CH₃, CH(CH₃)₂, —CN, NO₂, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂—C₁₋₄ alkyl, NR³SO₂-phenyl, S(O)ₚ—C₁₋₄ alkyl, S(O)ₚ-phenyl, and CF₃;
R⁵, at each occurrence, is selected from H, ═O, CH₃, CH₂CH₃, CH₂CH₂CH₃, CH(CH₃)₂, OR³, CH₂OR³, F, Cl, —CN, NO₂, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂—C₁₋₄ alkyl, NR³SO₂-phenyl, S(O)ₚ—C₁₋₄ alkyl, S(O)ₚ-phenyl, CF₃, phenyl substituted with 0-2 R⁶, naphthyl substituted with 0-2 R⁶, and benzyl substituted with 0-2 R⁶; and
R⁶, at each occurrence, is selected from H, OH, OR², F, Cl, CH₃, CH₂CH₃, CH₂CH₂CH₃, CH(CH₃)₂, —CN, NO₂, NR²R^{2a}, CH₂NR²R^{2a}, C(O)R^{2b}, CH₂C(O)R^{2b}, NR²C(O)R^{2b}, and SO₂NR²R^{2a}.

5. A compound according to claim 4, wherein:
P₁ is -G₁-G;
M₁ is -Z-A-B;
G is selected from: 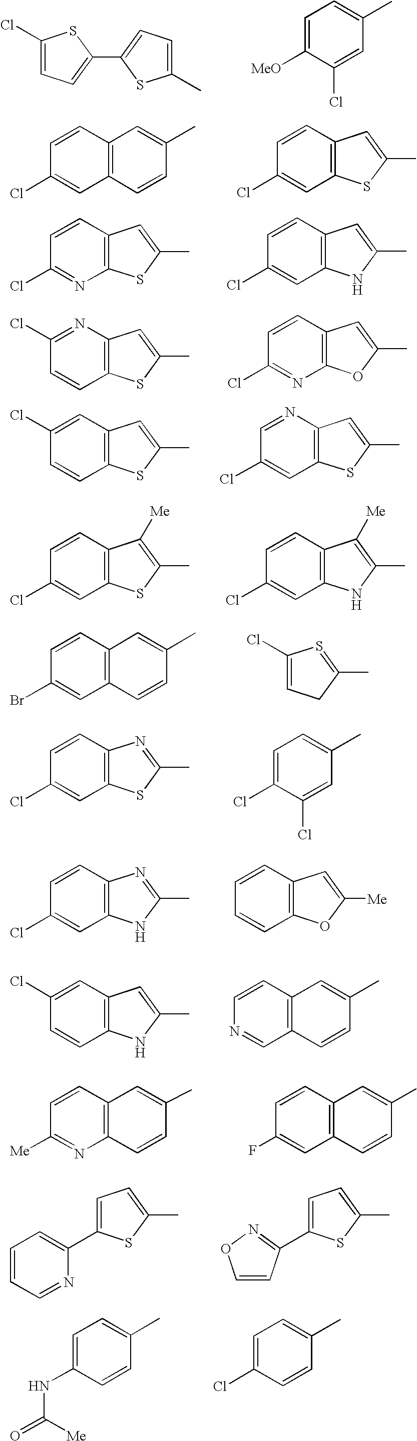 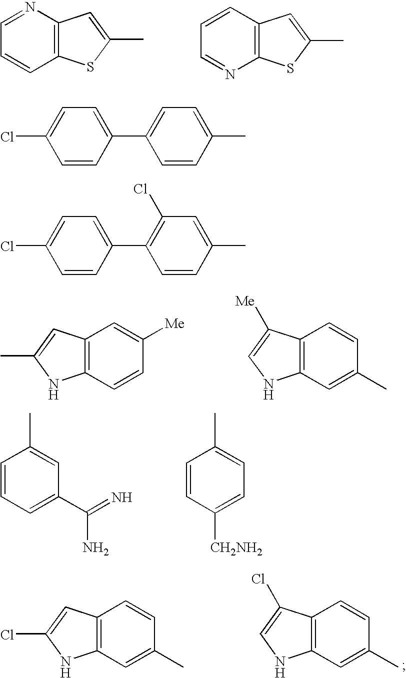
A is selected from the group: cyclohexyl, indolinyl, piperidinyl, phenyl, 2-pyridyl, 3-pyridyl, 2-pyrimidyl, 2-Cl-phenyl, 3-Cl-phenyl, 2-F-phenyl, 3-F-phenyl, 2-methylphenyl, 2-aminophenyl, and 2-methoxyphenyl;
B is selected from 2-oxo-pyridyl, 2-oxo-piperdinyl, 3-oxo-morpholinyl, phenyl, pyrrolidinyl, N-pyrrolidino-carbonyl, morpholinyl, N-morpholino-carbonyl, 1,2,3-triazolyl, imidazolyl, and benzimidazolyl, and is substituted with 0-1 R^{4a};
Z is selected from C(O), C(O)NH, NHC(O), NHC(O)NH, S(O)₂, SO₂NH, and NHSO₂, wherein the left side of Z is attached to ring M, provided that Z does not form a N—S, NCH₂N, NCH₂O, or NCH₂S bond with either group to which it is attached;
R^{1a}, at each occurrence, is selected from H, CH₃, CH₂CH₃, CH₂CH₂CH₃, CH₂F, CH₂Cl, Br, CH₂Br, —CN, CH₂CN, CF₃, CH₂CF₃, OCH₃, CH₂OH, C(CH₃)₂OH, CH₂OCH₃, NH₂, CH₂NH₂, NHCH₃, CH₂NHCH₃, N(CH₃)₂, CH₂N(CH₃)₂, CO₂H, COCH₃, CO₂CH₃, CH₂CO₂CH₃, SCH₃, CH₂SCH₃, S(O)CH₃, CH₂S(O)CH₃, S(O)₂CH₃, CH₂S(O)₂CH₃, C(O)NH₂, CH₂C(O)NH₂, SO₂NH₂, CH₂SO₂NH₂, NHSO₂CH₃, CH₂NHSO₂CH₃, pyridin-2-yl, pyridin-3-yl, pyridin-4-yl, pyridin-2-yl-N-oxide, pyridin-3-yl-N-oxide, pyridin-4-yl-N-oxide, imidazol-1-yl, CH₂-imidazol-1-yl, 4-methyl-oxazol-2-yl, 4-N,N-dimethylaminomethyl-oxazol-2-yl, 1,2,3,4-tetrazol-1-yl, 1,2,3,4-tetrazol-5-yl, CH₂-1,2,3,4-tetrazol-1-yl, and CH₂-1,2,3,4-tetrazol-5-yl, provided that R^{1a} forms other than an N-halo, N—S, or N—CN bond;
R², at each occurrence, is selected from H, CH₃, CH₂CH₃, CH₂CH₂CH₃, CH(CH₃)₂, phenyl substituted with 0-1 R^{4b}, benzyl substituted with 0-1 R^{4b}, and 5 membered aromatic heterocycle substituted with 0-1 R^{4b} and consisting of: carbon atoms and 1-4 heteroatoms selected from the group consisting of N, O, and S(O)ₚ;
R^{2a}, at each occurrence, is selected from H, CH₃, and CH₂CH₃;
alternatively, NR²R^{2a} forms a 5 or 6 membered saturated, partially saturated or unsaturated ring substituted with 0-1 R^{4b} and consisting of: 0-1 additional heteroatoms selected from the group consisting of N, O, and S(O)ₚ;
R^{2b}, at each occurrence, is selected from OH, OCH₃, OCH₂CH₃, CH₃, and CH₂CH₃;
R^{2c}, at each occurrence, is selected from OH, OCH₃, OCH₂CH₃, CH₃, and CH₂CH₃;
R^{4a} is selected from C₁₋₄ alkyl, CF₃, OR², CH₂OR², (CH₂)₂OR², NR²R^{2a}, CH₂NR²R^{2a}, (CH₂)₂NR²R^{2a}, S(O)ₚR⁵, SO₂NR²R^{2a}, and 1-CF₃-tetrazol-2-yl;
R^{4b}, at each occurrence, is selected from H, ═O, OR³, CH₂OR³, F, Cl, CH₃, CH₂CH₃, NR³R^{3a}, CH₂NR³R^{3a}, C(O)R³, C(O)OR^{3c}, NR³C(O)R^{3a}, C(O)NR³R^{3a}, SO₂NR³R^{3a}, NR³SO₂-phenyl, S(O)₂CH₃, S(O)₂-phenyl, and CF₃;
R⁵, at each occurrence, is selected from H, ═O, CH₃, CH₂CH₃, OR³, CH₂OR³, F, Cl, NR³R^{3a}, CH₂NR³R^{3a}, C(O)R³, C(O)OR^{3c}, NR³C(O)R^{3a}, C(O)NR³R^{3a}, SO₂NR³R^{3a}, NR³ SO₂—C₁₋₄ alkyl, NR³SO₂-phenyl, S(O)₂—CH₃, S(O)₂-phenyl, CF₃, phenyl substituted with 0-2 R⁶, naphthyl substituted with 0-2 R⁶, and benzyl substituted with 0-2 R⁶; and
R⁶, at each occurrence, is selected from H, OH, OR², F, Cl, CH₃, CH₂CH₃, NR²R^{2a}, CH₂NR²R^{2a}, C(O)R^{2b}, CH₂C(O)R^{2b}, NR²C(O)R^{2b}, and SO₂NR²R^{2a}.

6. A compound according to claim 5, wherein:
A is selected from the group: phenyl, 2-pyridyl, 3-pyridyl, 2-pyrimidyl, 2-Cl-phenyl, 3-Cl-phenyl, 2-F-phenyl, 3-F-phenyl, 2-methylphenyl, 2-aminophenyl, and 2-methoxyphenyl;
B is selected from the group: 2-oxo-pyridyl, 2-oxo-piperdinyl, 2-(aminosulfonyl)phenyl, 2-(methylaminosulfonyl)phenyl, N-pyrrolidino-carbonyl, 2-(methylsulfonyl)phenyl, 2-(N,N-dimethylaminomethyl)phenyl, 2-(N-methylaminomethyl)phenyl, 2-(N-ethyl-N-methylaminomethyl)phenyl, 2-(N-pyrrolidinylmethyl)phenyl, 1-methyl-2-imidazolyl, 2-methyl-1-imidazolyl, 2-(dimethylaminomethyl)-1-imidazolyl, 2-(methylaminomethyl)-1-imidazolyl, 2-(N-(cyclopropylmethyl)aminomethyl)phenyl, 2-(N-(cyclobutyl)aminomethyl)phenyl, 2-(N-(cyclopentyl)aminomethyl)phenyl, 2-(N-(4-hydroxypiperidinyl)methyl)phenyl, and 2-(N-(3-hydroxypyrrolidinyl)methyl)phenyl; and
G₁ is selected from: ═O, (CHR³)NHSO₂, (CHR³)NHC(O), (CHR³)NHC(O)NH, (CHR³)(CHR³)NHSO₂, (CHR³)(CHR³)NHC(O), (CHR³)SO₂, (CHR³)(CHR³)SO₂, (CR³R³)SO₂NH, (CHR³)(CHR³)SO₂NH, (CR³R³)C(O)NH, (CHR³)(CHR³)C(O)NH, NHC(O)NH, NHSO₂, NHSO₂CH₂, CH₂O, OCH₂, CH₂CH₂O, CH₂NH, CH₂CH₂NH, CH₂NHCH₂, NHCH₂, NHCH₂CH₂, CH₂C(O)NHSO₂, and CH₂SO₂NHC(O).

7. A compound according to claim 6, wherein the compound is of formula IIa or IIb: 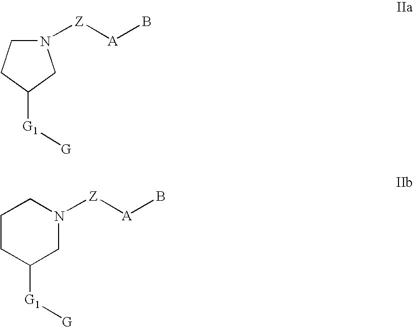
wherein: G₁ is selected from: ═O, CH₂NHSO₂, CH₂NHC(O), CH₂NHC(O)NH, CH₂CH₂NHSO₂, CH₂CH₂NHC(O), CH₂SO₂, CH₂CH₂SO₂, CH₂SO₂NH, CH₂CH₂SO₂NH, CH₂C(O)NH, CH₂CH₂C(O)NH, NHC(O)NH, NHSO₂, NHSO₂CH₂, CH₂O, OCH₂, CH₂CH₂O, CH₂NH, CH₂CH₂NH, CH₂NHCH₂, NHCH₂, NHCH₂CH₂, CH₂C(O)NHSO₂, and CH₂SO₂NHC(O).

8. A compound according to claim 1, wherein the compound is selected from:
6-chloro-naphthalene-2-sulfonic acid [1-(3,4,5,6-tetrahydro-2H-[1,4′]bipyridinyl-4-carbonyl)-pyrrolidin-3-ylmethyl]-amide;
4-methyl-2-pyridin-4-yl-thiazole-5-carboxylic acid [1-(6-chloro-naphthalene-2-sulfonyl)-pyrrolidin-3-ylmethyl]-amide;
5-chloro-3-methyl-benzo[b]thiophene-2-sulfonic acid [1-(2′-methanesulfonyl-biphenyl-4-carbonyl)-pyrrolidin-3-ylmethyl]-amide;
4′-chloro-biphenyl-3-sulfonic acid [1-(2′-methanesulfonyl-biphenyl-4-carbonyl)-pyrrolidin-3-yl]-amide;
6-chloro-naphthalene-2-sulfonic acid [1-(4-methyl-2-pyridin-4-yl-thiazole-5-carbonyl)-pyrrolidin-3-ylmethyl]-amide;
3′,4′-dichloro-biphenyl-4-sulfonic acid [1-(2′-methanesulfonyl-biphenyl-4-carbonyl)-pyrrolidin-3-ylmethyl]-amide;
2-(4-chloro-phenyl)-ethenesulfonic acid [1-(3,4,5,6-tetrahydro-2H-[1,4′]bipyridinyl-4-carbonyl)-pyrrolidin-3-ylmethyl]-amide;
5-pyridin-2-yl-thiophene-2-sulfonic acid [1-(2′-methanesulfonyl-biphenyl-4-carbonyl)-pyrrolidin-3-ylmethyl]-amide;
2-(3-chloro-phenyl)-ethenesulfonic acid [1-(3,4,5,6-tetrahydro-2H-[1,4′]bipyridinyl-4-carbonyl)-pyrrolidin-3-ylmethyl]-amide;
2-(5-bromo-thiophen-2-yl)-ethenesulfonic acid [1-(3,4,5,6-tetrahydro-2H-[1,4′]bipyridinyl-4-carbonyl)-pyrrolidin-3-ylmethyl]-amide;
2-phenyl-ethenesulfonic acid [1-(3,4,5,6-tetrahydro-2H-[1,4′]bipyridinyl-4-carbonyl)-pyrrolidin-3-ylmethyl]-amide;
6-chloro-benzo[b]thiophene-2-sulfonic acid [1-(3,4,5,6-tetrahydro-2H-[1,4′]bipyridinyl-4-carbonyl)-pyrrolidin-3-ylmethyl]-amide;
2-(5-bromo-thiophen-2-yl)-ethenesulfonic acid [1-(3,4,5,6-tetrahydro-2H-[1,4′]bipyridinyl-4-carbonyl)-pyrrolidin-3-(R)-ylmethyl]-amide;
(S)-6-bromo-naphthalene-2-sulfonic acid [1-(1-benzoyl-piperidine-4-carbonyl)-pyrrolidin-3-ylmethyl]-amide;
5′-chloro-[2,2′]bithiophenyl-5-sulfonic acid [1-(3,4,5,6-tetrahydro-2H-[1,4′]bipyridinyl-4-carbonyl)-pyrrolidin-3-yl]-amide;
5-chloro-benzo[b]thiophene-2-sulfonic acid [1-(3,4,5,6-tetrahydro-2H-[1,4′]bipyridinyl-4-carbonyl)-pyrrolidin-3-yl]-amide;
5-chloro-benzo[b]thiophene-2-sulfonic acid [1-(3,4,5,6-tetrahydro-2H-[1,4′]bipyridinyl-4-carbonyl)-pyrrolidin-2-ylmethyl]-amide;
5′-chloro-[2,2′]bithiophenyl-5-sulfonic acid [1-(3,4,5,6-tetrahydro-2H-[1,4′]bipyridinyl-4-carbonyl)-pyrrolidin-2-ylmethyl]-amide;
3,4,5,6-tetrahydro-2H-[1,4′]bipyridinyl-4-carboxylic acid [1-(5′-chloro-[2,2′]bithiophenyl-5-sulfonyl)-pyrrolidin-2-ylmethyl]-amide;
(R)-5-chloro-benzo[b]thiophene-2-sulfonic acid [1-(3,4,5,6-tetrahydro-2H-[1,4′]bipyridinyl-4-carbonyl)-pyrrolidin-3-ylmethyl]-amide;
(R)-2-(5-chloro-thiophen-2-yl)-ethenesulfonic acid [1-(3,4,5,6-tetrahydro-2H-[1,4′]bipyridinyl-4-carbonyl)-pyrrolidin-3-ylmethyl]-amide;
(R)-4′-(3-{[2-(5-chloro-thiophen-2-yl)-ethenesulfonylamino]-methyl}-pyrrolidine-1-carbonyl)-biphenyl-2-sulfonic acid amide;
(S)-2-(5-chloro-thiophen-2-yl)-ethenesulfonic acid {1-[1-(4-trifluoromethyl-pyrimidin-2-yl)-piperidine-4-carbonyl]-pyrrolidin-3-ylmethyl}-amide;
(S)-2-(5-chloro-thiophen-2-yl)-ethenesulfonic acid [1-(4-imidazol-1-yl-benzoyl)-pyrrolidin-3-ylmethyl]-amide;
(S)-2-(5-chloro-thiophen-2-yl)-ethenesulfonic acid {1-[1-(2-methyl-pyrimidin-4-yl)-piperidine-4-carbonyl]-pyrrolidin-3-ylmethyl}-amide;
(S)-2-(5-chloro-thiophen-2-yl)-ethenesulfonic acid {1-[1-(6-fluoro-quinolin-4-yl)-piperidine-4-carbonyl]-pyrrolidin-3-ylmethyl}-amide;
2-(5-chloro-thiophen-2-yl)-ethenesulfonic acid {1-[2-oxo-2-(2S-pyrrolidin-1-ylmethyl-pyrrolidin-1-yl)-acetyl]-pyrrolidin-3 S-ylmethyl}-amide;
2-(5-chloro-thiophen-2-yl)-ethenesulfonic acid {1-[2-oxo-2-(2S-pyrrolidin-1-ylmethyl-pyrrolidin-1-yl)-acetyl]-pyrrolidin-3R-ylmethyl}-amide;
(R)-2-(5-chloro-thiophen-2-yl)-ethenesulfonic acid {1-[1-(2-methyl-pyrimidin-4-yl)-piperidine-4-carbonyl]-pyrrolidin-3-ylmethyl}-amide;
(S)-2-(5-chloro-thiophen-2-yl)-ethenesulfonic acid {1-[2-(2-dimethylamino-methyl-piperidin-1-yl)-2-oxo-acetyl]-pyrrolidin-3-ylmethyl}-amide;
(R)-5′-chloro-[2,2′]bithiophenyl-5-sulfonic acid [1-(3,4,5,6-tetrahydro-2H-[1,4′]bipyridinyl-4-carbonyl)-pyrrolidin-3-ylmethyl]-amide
(R)-5′-chloro-[2,2′]bithiophenyl-5-sulfonic acid [1-(4-imidazol-1-yl-benzoyl)-pyrrolidin-3-ylmethyl]-amide;
(R)-5′-chloro-[2,2′]bithiophenyl-5-sulfonic acid {1-[1-(2-methyl-pyrimidin-4-yl)-piperidine-4-carbonyl]-pyrrolidin-3-ylmethyl}-amide;
5′-chloro-[2,2′]bithiophenyl-5-sulfonic acid [1-(2′-methanesulfonyl-biphenyl-4-carbonyl)-pyrrolidin-3-yl]-amide;
N-{4-[1-(2′-methanesulfonyl-biphenyl-4-carbonyl)-pyrrolidin-3-ylsulfamoyl]-phenyl}-acetamide;
5-isoxazol-3-yl-thiophene-2-sulfonic acid [1-(2′-methanesulfonyl-biphenyl-4-carbonyl)-pyrrolidin-3-yl]-amide;
5-(1-methyl-5-trifluoromethyl-1H-pyrazol-3-yl)-thiophene-2-sulfonic acid [1-(2′-methanesulfonyl-biphenyl-4-carbonyl)-pyrrolidin-3-yl]-amide;
6-chloro-thieno[2,3-b]pyridine-2-sulfonic acid [1-(2′-methanesulfonyl-biphenyl-4-carbonyl)-pyrrolidin-3-yl]-amide;
6-chloro-naphthalene-2-sulfonic acid [1-(2′-methanesulfonyl-biphenyl-4-carbonyl)-pyrrolidin-3-ylmethyl]-amide;
2-methanesulfonyl-N-[1-(2′-methanesulfonyl-biphenyl-4-carbonyl)-pyrrolidin-3-ylmethyl]-benzenesulfonamide;
N-[1-(2′-methanesulfonyl-biphenyl-4-carbonyl)-pyrrolidin-3-ylmethyl]-4-trifluoromethyl-benzenesulfonamide;
5-isoxazol-3-yl-thiophene-2-sulfonic acid [1-(2′-methanesulfonyl-biphenyl-4-carbonyl)-pyrrolidin-3-ylmethyl]-amide;
6-chloro-3-methyl-benzo[b]thiophene-2-sulfonic acid [1-(2′-methanesulfonyl-biphenyl-4-carbonyl)-pyrrolidin-3-yl]-amide;
6-chloro-3-methyl-benzo[b]thiophene-2-sulfonic acid [1-(3,4,5,6-tetrahydro-2H-[1,4′]bipyridinyl-4-carbonyl)-pyrrolidin-3-yl]-amide;
6-bromo-naphthalene-2-sulfonic acid [1-(3,4,5,6-tetrahydro-2H-[1,4′]bipyridinyl-4-carbonyl)-pyrrolidin-3-ylmethyl]-amide;
6-chloro-naphthalene-2-sulfonic acid [1-(3,4,5,6-tetrahydro-2H-[1,4′]bipyridinyl-4-carbonyl)-pyrrolidin-3-yl]-amide;
6-bromo-naphthalene-2-sulfonic acid [1-(3,4,5,6-tetrahydro-2H-[1,4′]bipyridinyl-4-carbonyl)-pyrrolidin-3-yl]-amide;
5-chloro-3-methyl-benzo[b]thiophene-2-sulfonic acid [1-(3,4,5,6-tetrahydro-2H-[1,4′]bipyridinyl-4-carbonyl)-pyrrolidin-3-ylmethyl]-amide;
5-chloro-benzo[b]thiophene-2-sulfonic acid [1-(3,4,5,6-tetrahydro-2H-[1,4′]bipyridinyl-4-carbonyl)-pyrrolidin-3-yl]-amide;
5′-chloro-[2,2′]bithiophenyl-5-sulfonic acid {1-[2-oxo-2-(2S-pyrrolidin-1-ylmethyl-pyrrolidin-1-yl)-acetyl]-pyrrolidin-3 S-ylmethyl}-amide;
(S)-5′-chloro-[2,2′]bithiophenyl-5-sulfonic acid [1-(2-oxo-2-pyrrolidin-1-yl-acetyl)-pyrrolidin-3-ylmethyl]-amide;
(R)-5′-chloro-[2,2′]bithiophenyl-5-sulfonic acid [1-(imidazole-1-carbonyl)-pyrrolidin-3-ylmethyl]-amide;
4′-chloro-biphenyl-3-sulfonic acid [1-(2′-methanesulfonyl-biphenyl-4-carbonyl)-pyrrolidin-3-yl]-amide;
5-bromo-6-chloro-pyridine-2-sulfonic acid [1-(2′-methanesulfonyl-biphenyl-4-carbonyl)-pyrrolidin-3-yl]-amide;
3′,4′-dichloro-biphenyl-4-sulfonic acid [1-(2′-methanesulfonyl-biphenyl-4-carbonyl)-pyrrolidin-3-ylmethyl]-amide;
5-pyridin-2-yl-thiophene-2-sulfonic acid [1-(2′-methanesulfonyl-biphenyl-4-carbonyl)-pyrrolidin-3-ylmethyl]-amide;
5-isoxazol-3-yl-thiophene-2-sulfonic acid [1-(2′-methanesulfonyl-biphenyl-4-carbonyl)-pyrrolidin-3-ylmethyl]-amide;
6-chloro-3-methyl-benzo[b]thiophene-2-sulfonic acid [1-(2′-methanesulfonyl-biphenyl-4-carbonyl)-pyrrolidin-3-yl]-amide;
benzo[1,2,5]oxadiazole-4-sulfonic acid [1-(2′-methanesulfonyl-biphenyl-4-carbonyl)-pyrrolidin-3-yl]-amide;
benzo[d]isoxazole-5-sulfonic acid [1-(2′-methanesulfonyl-biphenyl-4-carbonyl)-pyrrolidin-3-yl]-amide;
2-amino-pyrimidine-5-sulfonic acid [1-(2′-methanesulfonyl-biphenyl-4-carbonyl)-pyrrolidin-3-yl]-amide;
2-oxo-2,3-dihydro-1H-indole-5-sulfonic acid [1-(2′-methanesulfonyl-biphenyl-4-carbonyl)-pyrrolidin-3-yl]-amide;
6-chloro-3-methyl-benzo[b]thiophene-2-sulfonic acid [1-(3,4,5,6-tetrahydro-2H-[1,4′]bipyridinyl-4-carbonyl)-pyrrolidin-3-yl]-amide;
N-(1-(2-chloro-1H-indole-6-carbonyl)piperidin-3-yl)-4-(2-oxopyridin-1(2H)-yl)benzamide;
3-chloro-N-(1-(4-(2-oxopyridin-1(2H)-yl)benzoyl)pyrrolidin-3-yl)-1H-indole-6-carboxamide;
N-(1-(3-chloro-1H-indole-6-carbonyl)pyrrolidin-3-yl)-4-(2-oxopyridin-1(2H)-yl)benzamide;
3-chloro-N-(1-(4-(2-oxopyridin-1(2H)-yl)benzoyl)piperidin-3-yl)-1H-indole-6-carboxamide;
5-chloro-N-(1-(4-(2-oxopyridin-1(2H)-yl)benzoyl)piperidin-3-yl)thiophene-2-carboxamide; and
N-(1-(2-chlorothiophene-5-carbonyl)pyrrolidin-3-yl)-4-(2-oxopyridin-1(2H)-yl)benzamide N-(4-chlorophenyl)-3-(4-(2-oxopyridin-1(2H)-yl)benzamido)pyrrolidine-1-carboxamide
or a pharmaceutically acceptable salt form thereof.

9. A pharmaceutical composition, comprising: a pharmaceutically acceptable carrier and a therapeutically effective amount of a compound of claim 1 or a pharmaceutically acceptable salt form thereof.

10. A method for treating a thromboembolic disorder, comprising: administering to a patient in need thereof a therapeutically effective amount of a compound of claim 1 or a pharmaceutically acceptable salt form thereof.

11. A method according to claim 10, wherein the thromboembolic disorder is selected from the group consisting of arterial cardiovascular thromboembolic disorders, venous cardiovascular thromboembolic disorders, and thromboembolic disorders in the chambers of the heart.

12. A method according to claim 10, wherein the thromboembolic disorder is selected from unstable angina, an acute coronary syndrome, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from (a) prosthetic valves or other implants, (b) indwelling catheters, (c) stents, (d) cardiopulmonary bypass, (e) hemodialysis, or (f) other procedures in which blood is exposed to an artificial surface that promotes thrombosis.
